SEQUENCE LISTING (31 sequences)

SEQ ID NO: 1 - RNA, 75 nt, synthetic construct
  source 1..75: mol_type = other RNA
gtccccttcgtctagaggcccaggacaccgccctttcacggcggtaacaggggttcgaatcccctaggggacgcc

SEQ ID NO: 2 - RNA, 76 nt, synthetic construct
  source 1..76: mol_type = other RNA
gggtcgttagctcagttggtagagcagttgacttttaatcaattggtcgcaggttcgaatcctgcacgacccacca

SEQ ID NO: 3 - RNA, 75 nt, synthetic construct
  source 1..75: mol_type = other RNA
tggggtatcgccaagcggtaaggcaccggtttttgataccggcattccctggttcgaatccaggtaccccagcca

SEQ ID NO: 4 - RNA, 10 nt, synthetic construct
  source 1..10: mol_type = other RNA
ccctttcacg

SEQ ID NO: 5 - RNA, 15 nt, synthetic construct
  source 1..15: mol_type = other RNA
acttttaatcaattg

SEQ ID NO: 6 - RNA, 12 nt, synthetic construct
  source 1..12: mol_type = other RNA
tttttgataccg

SEQ ID NO: 7 - DNA, 12 bp, synthetic construct
  source 1..12: mol_type = other DNA
tttttgataccg

SEQ ID NO: 8 - DNA, 25 bp, synthetic construct
  source 1..25: mol_type = other DNA
ctacttgtacagctcgtccatgccg

SEQ ID NO: 9 - DNA, 899 bp, synthetic construct
  source 1..899: mol_type = other DNA
acaatgactgattacgattcgagctcggtacccggggatcctctagagatttaatacgactcactatagggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacctcaaggaggacggcaacatgctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagaatcgtcgacctgcaggcatgcaagcttggcactggccgtcgttttacaacgtcgtgactgggaaaaccctggcgttacccaacttaatcgccttgcagcacatcccc

SEQ ID NO: 10 - DNA, 12 bp, synthetic construct
  source 1..12: mol_type = other DNA
ccagggtgtcgc

SEQ ID NO: 11 - DNA, 12 bp, synthetic construct
  source 1..12: mol_type = other DNA
ccagggcgtcgc

SEQ ID NO: 12 - DNA, 11 bp, synthetic construct
  source 1..11: mol_type = other DNA
ttgcaaatcta

SEQ ID NO: 13 - DNA, 11 bp, synthetic construct
  source 1..11: mol_type = other DNA
ttgcatatcta

SEQ ID NO: 14 - DNA, 11 bp, synthetic construct
  source 1..11: mol_type = other DNA
ctgtaaactca

SEQ ID NO: 15 - DNA, 11 bp, synthetic construct
  source 1..11: mol_type = other DNA
ctgtatactca

SEQ ID NO: 16 - DNA, 11 bp, synthetic construct
  source 1..11: mol_type = other DNA
tttcaaatcaa

SEQ ID NO: 17 - DNA, 11 bp, synthetic construct
  source 1..11: mol_type = other DNA
tttcatatcaa

SEQ ID NO: 18 - DNA, 10 bp, synthetic construct
  source 1..10: mol_type = other DNA
cttccaaaca

SEQ ID NO: 19 - DNA, 84 bp, synthetic construct
  source 1..84: mol_type = other DNA
  modified_base 32: mod_base = i
  modified_base 33: mod_base = i
  modified_base 39: mod_base = i
ggcaacttggccgagtggttaaggcgaaagannagaaancttttgggctttgccgcgcaggttcgagtcctgcagttgtcgcca

SEQ ID NO: 20 - DNA, 86 bp, synthetic construct
  source 1..86: mol_type = other DNA
  modified_base 32: mod_base = i
  modified_base 33: mod_base = i
  modified_base 39: mod_base = i
  modified_base 41: mod_base = i
  modified_base 42: mod_base = i
  modified_base 43: mod_base = i
  modified_base 44: mod_base = i
  modified_base 49: mod_base = i
  modified_base 50: mod_base = i
  modified_base 51: mod_base = i
  modified_base 81: mod_base = i
ggcaacttggccgagtggttaaggcgaaagannagaaancnnnngggcnnnggcccgcgcaggttcgagtcctgcagttgncgcca

SEQ ID NO: 21 - RNA, 10 nt, synthetic construct
  source 1..10: mol_type = other RNA
ccctttcacg

SEQ ID NO: 22 - RNA, 12 nt, synthetic construct
  source 1..12: mol_type = other RNA
ccctttcacggc

SEQ ID NO: 23 - RNA, 13 nt, synthetic construct
  source 1..13: mol_type = other RNA
ccctttcacggcg

SEQ ID NO: 24 - RNA, 17 nt, synthetic construct
  source 1..17: mol_type = other RNA
ttgacttttaatcaatt

SEQ ID NO: 25 - RNA, 18 nt, synthetic construct
  source 1..18: mol_type = other RNA
ttgacttttaatcaattg

SEQ ID NO: 26 - RNA, 21 nt, synthetic construct
  source 1..21: mol_type = other RNA
ttgacttttaatcaattggtc

SEQ ID NO: 27 - RNA, 15 nt, synthetic construct
  source 1..15: mol_type = other RNA
acttttaatcaattg

SEQ ID NO: 28 - RNA, 18 nt, synthetic construct
  source 1..18: mol_type = other RNA
acttttaatcaattggtc

SEQ ID NO: 29 - RNA, 10 nt, synthetic construct
  source 1..10: mol_type = other RNA
ccctttcacg

SEQ ID NO: 30 - RNA, 11 nt, synthetic construct
  source 1..11: mol_type = other RNA
tttttgatacc

SEQ ID NO: 31 - RNA, 12 nt, synthetic construct
  source 1..12: mol_type = other RNA
tttttgataccg

(19) United States
(12) Patent Application Publication
(10) Pub. No.: US 2024/0376069 A1
(43) Pub. Date: Nov. 14, 2024

(54) COMPOUNDS AND METHODS FOR LABELING LIPIDATED NUCLEOTIDES

(71) Applicant: The Research Foundation for The State University of New York, Albany, NY (US)

(72) Inventors: Jia SHENG, Schenectady, NY (US); Rui WANG, Wuhan (CN)

(73) Assignee: The Research Foundation for The State University of New York, Albany, NY (US)

(21) Appl. No.: 18/661,155

(22) Filed: May 10, 2024

Related U.S. Application Data
(60) Provisional application No. 63/465,425, filed on May 10, 2023.

(51) Int. Cl. C07D 401/04; G01N 33/533

(52) U.S. Cl. CPC C07D 401/04; G01N 33/533

(57) ABSTRACT

Provided is provided is a compound of Formula I

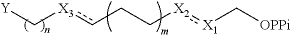

and a method of forming a tagged 2-thiouridine tRNA by contacting it with a compound of Formula I, wherein the compound may include a label such as a fluorescent label. Also provided is a method of forming a tagged RNA, comprising contacting a prenylated tRNA or an N⁶-isopentenyladenosine-prenylated RNA with a 4-phenyl-1,2,4-triazoline-3,5-dione activated fluorescence label. Also provided is a method of sequencing a polyribonucleotide comprising a prenyl group.

### CROSS-REFERENCE TO RELATED APPLICATIONS

This application claims benefit of priority from U.S. Provisional Patent Application No. 63/465,425, filed May 10, 2023, the entire content of which is incorporated herein by reference.

### GOVERNMENT RIGHTS STATEMENT

This invention was made with Government support under grant numbers CHE1845486 and MCB1715234 awarded by the National Science Foundation. The Government has certain rights in the invention.

### SEQUENCE LISTING

The instant application contains an electronic sequence listing. The contents of the electronic sequence listing H2998864.xml; Size: 34,034 bytes; and Date of Creation: May 10, 2024 is herein incorporated by reference in its entirety.

### BACKGROUND

Nucleic acids are the basic components of all life and are essential for cell activities such as division, migration, apoptosis, and proliferation. To modify RNAs' structure and function, more than 170 chemical alterations are commonly added to both coding and non-coding regions. RNA modifications such as methylation, acetylation, phosphorylation, glycosylation, palmitoylation, and prenylation not only diversify RNA structure but also play important functional roles in normal cell growth and development as well as response regulations to environmental stress. Furthermore, chemical groups that modify the canonical nucleobases have been shown to involve in regulating the interactions of RNAs with other biomacromolecules such as proteins, lipids, sugars, and other forms of nucleic acids. Dysfunctional RNA alterations are connected to a variety of human diseases, including cancer and viral infections as well as developmental abnormalities and cancer, extending the networks of interaction. Common chemical modifications include as m⁶A and m⁵C, on various RNA species, playing crucial functions in biological processes.

A significant hydrophobic, C₁₀H₁₅ lipid moiety modification has been identified on the wobble position of some specific bacterial tRNAs as a geranyl group attached to the sulfur atom at position 2 of uridine (ges²U). This geranyl group modifies about 0.4% of tRNAs, specifically for lysine, glutamine and glutamic acid in Escherichia coli, Enterobacter aerogenes, Pseudomonas aeruginosa, and Salmonella enterica serovar Typhimurium. The geranyl modification improves tRNA translation fidelity and reduces frameshifting errors, yielding tRNA that preferentially base pairs with G over A, resulting in a significant improvement in base pairing discrimination and codon recognition. Geranylated tRNA may also be a transient intermediate in the selenation process, another important tRNA modification pathway in bacteria.

In general, it has been discovered that lipid-like hydrophobic prenyl groups, such as geranyl, farnesyl, and geranyl-geranyl, change both proteins and RNAs with important activities. Many organic substances, including protein enzymes like KRAS and caryophyllene are prenylated. The farnesyl and geranylgeranyl groups on cysteine in the CAAX motif of RAS superfamily proteins are important for signaling transduction in anchoring the proteins to the cell membrane. 2-selenouridine synthase (SelU), a conserved enzyme essential for efficient bacterial growth, mediates geranylation in ges²U, but understanding of the fundamental regulations that govern geranylation process and its functions is still relatively restricted. Nothing is known about the regulatory relationships between geranylation and other reader, writer, and eraser enzymes. Biochemical tools that allow for the labeling of lipidated RNAs to enable the study of hydrophobic modifications in normal and diseased biological processes are lacking. Incorporating similar lipid analogs into tRNAs could also provide a useful platform for the development of new molecular tools for labeling, detecting, and isolating specific tRNAs for in vitro, in situ, and in vivo applications.

Hydrophobic prenyl groups (attached to i⁶A ribonucleotide analogs introduced by the Mia family enzyme) have also been discovered in tRNAs. These modifications are usually found in the position 37 of anticodon stem loops with great biological significance in enhancing base pairing specificity, codon recognition fidelity and translational efficiency. Prenyl modification of tRNA affects plant root growth. In addition, they have been actively involved in a variety of human diseases such as mitochondrial respiratory chain defects, Alzheimer's disease, breast cancer and diabetes. The prenyl groups including terpenes compounds have also been known as the fundamental feedstock in a wide range of biological processes that play key roles in metabolism and human diseases. The transcriptome-wide mapping of 2-methylthio-i⁶A (ms²i⁶A), a common i⁶A analog, has been achieved through chemoselective bioconjugation of the methylthio group. Elucidating the biological significance of these prenyl-modified RNAs remains very challenging due to the lack of genome wide detection and sequencing tools.

Antigen-antibody specificity is one of the main methodologies to identify and profile RNA modifications, and chemical pulldown methods have been used to study modified residues in RNAs such as hm⁵C, f⁵C, and pseudouridine, etc. In contrast to using antibodies to recognize specific epitope of antigen, chemical pulldown strategies target defined functionalities on RNAs and employ highly selective chemical probes to tag the RNAs containing these modifications within the complex cell environment. However, there remains a need for more general approaches targeting the prenyl-group to better profile these modifications

The present disclosure is directed to overcoming these and other deficiencies in the art.

### SUMMARY

In an aspect, provided is a compound of Formula I:

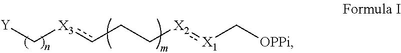

wherein X₁ may be —C(═O)— or —CH₂—, each [symbol] may be, independently, a single bond or a double bond, X₂ may be —NH— or —C(CH₃)—, m may be 0 or 1, X₃ may be —CH₂— or —C(CH₃)—, n may be an integer of from 0 to 8, and Y may be N₃ or

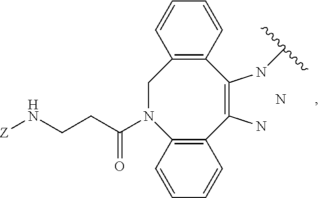

wherein Z may be a fluorescent label. n may be an integer of from 1 to 8. m may be 1. m may be 1 and n may be 8. m may be 0 and n may be 1.

Each of X₂ and X₃ may be —C(CH₃)— and each [symbol] may be a double bond. X₁ may be —CH₂—, X₂ may be —C(CH₃)—, each [symbol] may be a double bond, m may be 1, X₃ may be —C(CH₃)—, and n may be 1; or X₁ may be —C(═O)—, X₂ may be —NH—, each [symbol] may be a single bond, m may be 0, X₃ may be —CH₂—, and n may be 1. Z may be a fluorescent label selected from Cy5, FITC, and BODIPY.

In another aspect, provided is a method of forming a tagged tRNA, including contacting a 2-thiouridine tRNA with a compound of Formula I:

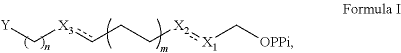

wherein X₁ may be —C(═O)— or —CH₂—, each [symbol] may be, independently, a single bond or a double bond, X₂ may be —NH— or —C(CH₃)—, m may be 0 or 1, X₃ may be —CH₂— or —C(CH₃)—, n may be an integer of from 0 to 8, and Y may be an azide or

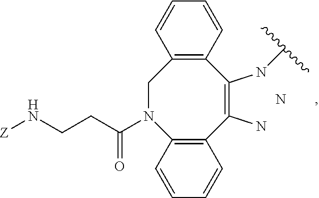

wherein Z may be a fluorescent label; and a step for covalently attaching the compound of Formula I to the 2-thiouridine tRNA. n may be an integer of from 1 to 8. m may be 1. m may be 1 and n may be 8. m may be 0 and n may be 1.

Each of X₂ and X₃ may be —C(CH₃)— and each [symbol] may be a double bond. X₁ may be —CH₂—, X₂ may be —C(CH₃)—, each [symbol] may be a double bond, m may be 1, X₃ may be —C(CH₃)—, and n may be 1; or X₁ may be —C(═O)—, X₂ may be —NH—, each [symbol] may be a single bond, m may be 0, X₃ may be —CH₂—, and n may be 1.

Y may be an azide, and forming the tagged tRNA may further include contacting the azide with a DBCO-activated fluorescent label to cause a click-chemistry reaction therebetween. Y may be

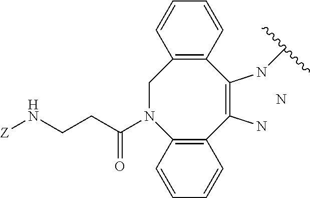

and Z may be a fluorescent label.

The step for covalently attaching the compound of Formula I to the 2-thiouridine tRNA may occur intracellularly. The step for covalently attaching the compound of Formula I to the 2-thiouridine tRNA may occur extracellularly. Z may be a fluorescent label selected from Cy5, FITC, and BODIPY.

In another aspect, provided is a method of forming a tagged tRNA, comprising contacting a prenylated tRNA with a 4-phenyl-1,2,4-triazoline-3,5-dione activated fluorescence label to cause an ene reaction therebetween.

In another aspect, provided is a method of adding a label to a ribonucleotide including a prenyl group, comprising contacting the ribonucleotide with an activated label, wherein the ribonucleotide includes an N⁶-isopentenyladenosine-prenylated RNA and the activated label includes the label coupled to 4-phenyl-1,2,4-triazoline-3,5-dione, and wherein the contacting comprises forming a covalent attachment between the prenyl group and the 4-phenyl-1,2,4-triazoline-3,5-dione. The N⁶-isopentenyladenosine-prenylated RNA may include a polynucleotide. The label may include a fluorine. The label may include a fluorescent label. The fluorescent label may be selected from Cy5, FITC, and BODIPY.

In another aspect, provided is a method of sequencing a polyribonucleotide comprising a prenyl group, including contacting the prenyl group of the ribonucleotide with I₂, wherein the ribonucleotide includes an N⁶-isopentenyladenosine-prenylated RNA and contacting the prenyl group of the ribonucleotide with I₂ includes causing a cyclisation reaction of the prenyl group, reverse transcribing the polyribonucleotide to form a cDNA of the polyribonucleotide, and sequencing the cDNA of the polyribonucleotide. The method may further include reverse transcribing a control RNA having the same sequence as the polyribonucleotide to form a control cDNA, wherein the control RNA was not contacted with I₂, sequencing the control cDNA, and detecting an N⁶-isopentenyladenosine in the polyribonucleotide wherein detecting comprises identifying a C or G in a position of the cDNA of the polyribonucleotide corresponding to a position of an A in the control cDNA.

### BRIEF DESCRIPTION OF THE DRAWINGS

These and other features, aspects, and advantages of the present disclosure will become better understood when the following detailed description is read with reference to the accompanying drawings, wherein:

FIG. 1 shows Labeling of geranylated RNA and identifying “reader” proteins. Left: The chemical structure of the geranyl modification on tRNA (represented as a ribbon). Right: Schematics of the fluorescent labeling on geranylated RNA and its reader proteins. R═H (ges²U, 1), 5-methylaminomethyl (mnm⁵ges²U, 2), or 5-carboxylmethyl amidomethyl (cmnm³ges²U, 3).

FIG. 2 shows Chemical structures of geranyl pyrophosphate variants. Chemical structures of geranyl pyrophosphate variants Ge1-7. Ge1 for positive control. Ge2-3 for nucleosides analysis experiment. Ge5-7 for fluorescent labeling studies. Ge4 for photo-mediated cross-linking experiment.

FIG. 3 shows Structure prediction of the core domain in RNA geranylation enzyme SelU. (A) Ribbon diagram for the predicted structure of SelU based on the prediction from AlphaFold. (B) Predicated Cys97 interaction network. (C) Analysis of SelU critical sites Gly67. (D) Plausible mechanism of tRNA geranylation catalyzed by SelU enzyme in the presence of magnesium and prenyl pyrophosphate. The pyrophosphate structural comparisons between Ge5, Ge6 and the natural substrate Ge1 were also presented. (E) 3D structural comparisons of the pyrophosphate variants Ge1, Ge5 and Ge6. (F) Molecule docking result from Autodock Vina demonstrated the structural similarities of SelU-ligand complexes with Ge1, Ge5 and Ge6.

FIG. 4 shows Western blotting analysis of interacted proteins of prenylated tRNA using pyrophosphate Ge4. Group1, Ge4 was added to lysate; Group2, in the absence of Ge4; Group3, Ge4 was added to lysate in the presence of SelU.

FIG. 5 shows SelU-catalyzed tRNA geranylation with pyrophosphate Ge5 as substrate, the in-gel fluorescent image results.

FIG. 6 shows SelU-catalyzed tRNA geranylation with pyrophosphate Ge6 as substrate, the in-gel fluorescent image results.

FIG. 7 shows SelU-catalyzed tRNA geranylation with pyrophosphate Ge5-7 as substrate, the in-gel fluorescent image results. Ge5-6 and Ge7 substrates in the fluorescent labeling study. PTAD-DBCO-Cy5 was used for Ge5 or Ge6. BODIPY was used for Ge7. Blue channel (E.X. 492 nm. E.M. 507 nm), red channel (E.X. 649 nm. E.M. 660 nm) and green channel (nm).

FIGS. 8A-8D show (A) tRNA^{Gln, Glu, Lys} modified with pyrophosphate G5 (10 μM) was visualized via strain-promoted alkyne-azide cycloaddition (SPAAC) reaction in the presence of DBCO-diSulfo-Cy5 (10 μM). (B) tRNA^{Gln,Glu,Lys} modified with pyrophosphate G6 (10 μM) was visualized via SPAAc reaction with DBCO-diSulfo-Cy5 (10 μM) along with SYBR staining. (C) tRNA^{Gln, Glu, Lys} modified with pyrophosphate G7 (10 μM) was visualized via SPAAC reaction using BODIPY-R6G (10 μM). The top panel (SYBR staining) loaded with equivalent amounts of tRNA^{Gln,Glu,Lys} with and without geranyl pyrophosphates (Ge5-7). (D) The chemical structures of DBCO-diSulfo-Cy5 on the left and BODIPY-R6G on the right. All in-gel fluorescence assays were analyzed on 3% PAGE gel. At least three duplicates were performed, and Zeiss Confocal Image was used to measure in-gel fluorescent intensity.

FIG. 9 shows Structure details of tRNA^{Glu}_{UUC} from E. coli and its cleavable G site by use of RNase T1. (5′-GUCCC CUUCG UCUAG AGGCC CAGGA CACCG CCCUU UCACG GCGGU AACAG GGGUU CGAAU CCCCU AGGGG ACGCC A-3′, 24.441 kDa). SEQ ID NO: 1.

FIG. 10 shows Structure details of tRNA^{Lys}_{UUU} from E. coli and its cleavable G site by use of RNase T1. (5′-GGGUC GUUAG CUCAG UUGGU AGAGC AGUUG ACUUU UAAUC AAUUG GUCGC AGGUU CGAAU CCUGC ACGAC CCACC A-3′, 24,441 kDa). SEQ ID NO: 2.

FIG. 11 shows Structure details of tRNA^{Gln}_{UUU/CUG} from E. coli and its cleavable G site by use of RNase T1. (5′-UGGGG UAUCG CCAAG CGGUA AGGCA CCGGU UUUUG AUACC GGCAU UCCCU GGUUC GAAUC CAGGU ACCCC AGCCA-3′, 24,124 kDa). SEQ ID NO: 3.

FIGS. 12A-12D show Direct labelling of tRNAs in E. coli. with diSulfo-Cy5-DBCO-PTAD. tRNAs (1.0 mg from E. coli, Roche) were reacted with diSulfo-Cy5-DBCO-PTAD (1.0 mM) for 5 min. 3% Agarose gel was used to visualize the geranylated tRNAs and confirmed by MALDI-TOF. Total tRNAs reacted directly with the probe diSulfo-Cy5-DBCO-PTAD (10 μM). (A) Fluorescent labelling of geranylated tRNAs by Ene-ligation. (B) tRNA^{Gln, Glu, Lys} extracted from E. coli. reacted with pre-established probe followed by SPAAC reaction with diSulfo-Cy5-DBCO-PTAD (10 μM). The top panel (SYBR staining) showed the equivalent amounts of tRNA^{Gln, Glu, Lys} with and without probe were loaded. (C) Chemical structure of diSulfo-Cy5-DBCO-PTAD dye (In situ generated by NBS). (D) Illustrated the HRMS mass 25724.9355 (cnm⁵ges²U-tRNA^{Glu/Lys}, 2‰) and 25417.4251 (cmnm⁵ges²U-tRNA^{Gln}, 2‰). The mass spectra were analyzed on a Shimadazu AXIMA Performance-MALDI TOF/TOF.

FIGS. 13A-13C show Fluorescence labelling of E. coli. tRNA^{Lys}-5S in 293T cells. (A) Overall illustrations of the fluorescence labelling of prenylated RNA in vivo. (B) Top panel: Transfection of fusion plasmid SelU-EGFP with transcript 5S in the presence of pyrophosphate Ge1 (1 mM), followed by the treatment of PTAD-DBCO-Cy5 probe (50 μM). Bottom panel: Transfection of fusion plasmid SelU-EGFP without transcript 5S in the presence of pyrophosphate Ge1 (1 mM) and PTAD-DBCO-Cy5 treatment. Green fluorescence (left), Cy5 fluorescence (second), merged (third), and DAPI fluorescence (nuclear staining; right). (C) Transfection of fusion plasmid Sell-pCMV-Myc-EGFP-5s in the presence of pyrophosphate Ge6, followed by the treatment of DBCO-Cy5 probe (5 μM) via click reaction. Imaging of HEK293T cells harboring the SelU-p (MV-Myc-EGFP-5s plasmid (top line, both red and green fluorescence), SelU-pCMV-Myc-EGFP plasmid (second line, only green fluorescence in the absence of transcript 5s); p (MV-Myc-EGFP (bottom line, no green/red fluorescence in the absence of SelU). Green fluorescence (left), Cy5 fluorescence (second), merged (third), and DAPI fluorescence (nuclear staining; right). DAPI=4′,6-dianidino-2-phenylindole. Scale bar: 20 μm.

FIG. 14 shows Confocal imaging investigations of fluorescent labeling of tRNA harboring 5s-pCMV3 and SelU-pCMV-Myc-EGFP in HEK293T cell lines using probe PTAD-DBCO-Cy5 in the presence of pyrophosphate Ge1 (top). Control assays are: in the absence of 5s-pCMV3 (bottom). Scale bar: 20 μm.

FIG. 15 shows Confocal imaging investigations of fluorescent labeling of tRNA harboring 5s-pCMV3 and SelU-pCMV-Myc-EGFP in HEK293T cell lines using probe DBCO-Cy5 in the presence of Ge6 (top). Control assays are: in the absence of 5s-pCMV3 (bottom, left) or in the absence of SelU-pCMV-Myc-EGFP (bottom, right). Scale bar: 20 μm.

FIG. 16 shows Fluorescent dyes used herein for tagging of geranylated RNA.

FIG. 17 shows Chemical structures of fluorescent probes used.

FIGS. 18A-18E show Overview of prenylated RNA residues as well as the labeling and profiling strategy described in this work. (A) Structures of prenylated nucleosides. (B) Plausible biogenetic pathways for prenylated RNAs. (C) Fluorination of i⁶A-incorporated RNA. (D) Labeling and enrichment methods proposed in this work. (E) Iodine-mediated cyclization and reverse transcription (IMCRT) method to profile i⁶A residues in cell.

FIGS. 19A-19C show the reactions of i⁶A (4) with various functionalities. (A) Reaction of i⁶A (4) with PTAD (5) at room temperature. (B) Proton NMR was used to monitor the reaction of i⁶A (4, 0.03 mmol, 30 mM) with PTAD (0.15 mmol) in CD₃CN/D₂O (v/v, 1/1) at room temperature. The rapid reaction resulted in approximately 50% conversion yield within 5 minutes (peak at 5.5 ppm, triplet, is assigned to alkenyl site in prenyl group of i⁶A). From top to bottom: 4 hours, 2 hours, 1 hour, 30 minutes, 5 minutes and 0 minute. (C) Chemical structures of the green fluorescent probe PTAD-DBCO-FITC (8) and the red fluorescent probe PTAD-DBCO-Cy5 (10).

FIG. 20 shows an i Illustration of the construction of transcription system of chimeric T7P-EGFP gene in vitro (T7 promoter sequence SEQ ID NO: 7; EGFP Gene sequence nucleotides 72-788 of SEQ ID NO: 9).

FIG. 21 shows Detection of the transcripts of T7P-EGFP. Lane 1: T7P-EGFP template. Lane 2: HeLa genome. T7-RNAp-EGFP represents T7P-EGFP herein.

FIG. 22 shows transcription system in vitro. Lane 1: ladder. Lane 2-3 were positive control assays. Lane 2 (rNTP mixtures, 2.0 μl, 100 mM, separately). Lane 3 (A/C/G/UTP, 0.5 μl, 100 mM, separately). Lane 5-8 (C/G/UTP, 0.5 μl, 100 mM, separately) and varied concentrations of i⁶ATP (0.5, 1.0, 1.5 and 2.0 μl, 100 mM). 1% agarose gel electrophoresis was used to monitor the transcripts.

FIG. 23 shows Optimization of the transcription efficiency in vitro. P. C. represents positive control assay, rNTP mixtures are A/C/U/GTP (0.5 μl, 100 mM, separately). All experiments were used mixtures with rNTP (A/C/U/GTP, 0.5 μl, 100 mM, separately) and varied concentrations of i⁶ATP: 0.5, 1.0, 2.0, 4.0, 6.0, 8.0 μl (100 mM). Transcripts were run via 1% agarose gel electrophoresis.

FIG. 24 shows: Optimization of the transcription efficiency in vitro. P. C. represents Positive Control assay, rNTP mixtures are A/C/U/GTP (0.5 μl, 100 mM, separately). All experiments were used mixtures with rNTP (A/C/U/GTP, 0.5 μl, 100 mM, separately) and varied concentrations of i⁶ATP: 0.5, 1.0, 2.0, 4.0, 6.0, 8.0 μl (100 mM). Transcripts were run via 1% agarose gel electrophoresis. Control1 is a negative control without template, and Control2 is a negative control without substrate. T7-RNAp-EGFP represents T7P-EGFP herein.

FIG. 25A-25C show: The structure of the intracellular transcription system catalyzed by T7 RNA polymerase. Top A, the T7 RNA polymerase transcription system guided by the CMV promoter and the EGFP transcription system guided by the T7 promoter are constructed in the same vector. Bottom B and C, the T7 RNA polymerase transcription system guided by the CMV promoter and the EGFP transcription system guided by the T7 promoter were constructed into two transfection systems respectively.

FIGS. 26A-26B show: Western-blotting detection the protein expression of T7 RNA polymerase and EGFP. FIG. 26A. Transfect the (MV-T7 RNA polymerase transcription system into HeLa cells, and detection of the protein expression of T7 RNA polymerase after 36 hours. The T7 RNA polymerase has a FLAG tag. FIG. 26B. HeLa cells were transfected with CMV-17 RNA polymerase and 77 RNA polymerase-IRES2-EGFP transcription system, and CMV-17 RNA polymerase and T7 RNA polymerase-IRES2-EGFP combined system, respectively. The expression of EGFP was detected 36 hours later. T7 RNAp represents T7 RNA polymerase.

FIG. 27 shows: Detection of T7 RNA polymerase transcription system catalyzing the transcription and expression of EGFP fluorescent protein in Hela cells. The (MV-T7 RNA polymerase and T7 RNA polymerase-IRES2-EGFP systems were transfected into HeLa cells, and the two systems were transfected together and the integrated system was transfected. After 36 hours of the transfection, the expression of EGFP green fluorescent protein was detected under a fluorescence microscope. Scale bar 50 μM. T7 RNAp represents T7 RNA polymerase herein. Scale bar: 50 um.

FIGS. 28A-28F show: Investigation of the synthesized i⁶A (4)-incorporated RNAs in eukaryotic cells and its fluorescent labeling. (A) Expression level of EGFP green fluorescent protein initiated by the T7 RNA polymerase transcription system in HeLa cells. A1-A4 represent the transfection of CMV-T7 RNA polymerase, T7pro-IRES2-EGFP, CMV-T7 RNA polymerase with T7pro-IRES2-EGFP, and integrated CMV-T7 RNA polymerase-T7 pro-IRES2-EGFP respectively. Only co-transfection or integrated systems of CMV-T7 RNA polymerase and T7 pro-IRES2-EGFP displayed green fluorescent signals. (B) Detection of the green fluorescent signal in i⁶A-incorporated RNAs using PTAD-DBCO-FITC (8, 0.1 mM). (C) Goldview dye staining of i⁶A-incorporated RNAs. (D) Fluorescent labeling of i⁶A-incorporated RNAs using the well-designed PTAD-DBCO-Cy5 (10, 0.1 mM). (E) Fluorescent labeling assays of RNAs harboring i⁶A conducted through reverse transcription. “−”, without PTAD-DBCO-Cy5 treatment; “+”, with PTAD-DBCO-Cy5 treatment. (F) Red fluorescent imaging of PTAD-DBCO-Cy5 (10)-labeled i⁶A-RNAs in a gel. Bar scale: 50 μm.

FIG. 29 shows: Cytotoxic assays using various concentration of i⁶A (4) in 12 hours or 24 hours.

FIG. 30 shows: Investigation of i⁶A's role in RNA transcription within eukaryotic cells. Following a 24-hour incubation of HeLa cells with DMEM medium containing 200 μM, 400 μM, and 800 μM of i⁶A (4), total RNA was extracted and labeled with PTAD-DBCO-FITC (8) to assess the fluorescence intensity of PTAD-DBCO-FITC (8).

FIG. 31 shows: Gel Electrophoresis Analysis of Transcripts Containing i⁶A (4). Left: UV Monitoring. Right: GoldView™ Staining Monitoring.

FIG. 32 shows: Directly fluorescent labeling of prenylated tRNA from E. coli using PTAD-DBCO-Cy5 (10).

FIGS. 33A-33C show: Investigation of the chemical transformations of prenylated nucleoside i⁶A (4) with Iodine (I₂). (A) Reaction of i⁶A (4, 0.15 mmol) with iodine (0.45 mmol) at room temperature for 5 minutes. (B) Comparative analysis using proton nuclear magnetic resonance (¹H NMR, DMSO-d₆, 400 MHz) for the reaction of i⁶A (4) and I₂ with and without sodium persulfate. Bottom: ¹H NMR spectrum of pure i⁶A (4). Middle: ¹H NMR spectrum of pure i⁶A (4) following exclusive treatment with I₂. Top: ¹H NMR spectrum of pure i⁶A (4), initially treated with I₂ and subsequently treated with sodium persulfate. (C) DFT calculations for the possible product of the reaction of i⁶A (4) with I₂. All the calculations were performed using the Gaussian 16, Revision B.01 program.

FIG. 34 shows: DFT calculations of the reaction of i⁶A (4) with iodine. Deiodination process was calculated to be very fast. In contrast, the iodine epoxide intermediate was elusive.

FIG. 35 shows: Outcomes of mutant assays involving RNA with incorporated i⁶A (4).

FIGS. 36A-36C2 show: Detection of i⁶A sites in RNA at a single-base resolution. (A) Detection scheme for i⁶A modification sites in RNA by employing i⁶ATP in in vitro studies. Schematic of i⁶A insertion at a specific RNA site and chemical-induced mutation with I₂ treatment. In the graph, ‘X’ denotes a mixture containing 70% molar concentration of ATP and 30% molar concentration of i⁶ATP. ‘Y’ signifies the reaction product derived from X following treatment with Iodine (I₂). CCAGGGTGTCGC SEQ ID NO: 10; CCAGGGCGTCGC SEQ ID NO: 11. (B) Insertion of i⁶ATP at a specific site of transcribed RNA followed by chemical mutation after I₂ treatment. A comparative analysis of i⁶A-incorporated RNA with and without iodine treatment showed that I₂ treatment is a powerful tool for detecting i⁶A in nucleic acids. (C) Determination of mRNA labeling efficiency by i⁶ATP under in vitro transcription conditions.

FIGS. 37A-37D show: IMCRT tRNA-seq results. Analyses showing the high quality of IMCRT tRNA-seq data in S. cerevisiae. (A) Read alignment statistics of yeast tRNA with or without treatment. (B) Correlation between biological replicates. Heat-map of Pearson's correlation from all sequencing data compared against each other, represented by a colored field ranging from blue (0.7) to red (1). (C) Scatter plot showing the correlation of cytosolic tRNA abundance measured by IMCRT tRNA-seq with tRNA gene copy number without I₂ treatment. Pearson's R=0.67, p<6 e-8, n=52. (D) Scatter plot of tRNA gene copy number vs tRNA abundance after I₂ treatment. Pearson's R=0.62, p<9 e-7, n=52.

FIGS. 38A-38D show: Analyses showing the high quality of IMCRT tRNA-seq data in S. cerevisiae. (A) Read alignment statistics of yeast tRNA with or without treatment. (B) Correlation between biological replicates. Heat-map of Pearson's correlation from all sequencing data compared against each other, represented by a colored field ranging from blue (0.7) to red (1). (C) Scatter plot showing the correlation of cytosolic tRNA abundance measured by IMCRT tRNA-seq with tRNA gene copy number without I₂ treatment. Pearson's R=0.67, p<6 e-8, n=52. (D) Scatter plot of tRNA gene copy number vs tRNA abundance after I₂ treatment. Pearson's R=0.62, p<9 e-7, n=52.

FIGS. 39A-39D show: Detection of i⁶A modified nucleotides in yeast mitochondrial (mt) tRNAs. (A) Read coverage of the above-indicated mt-tRNAs with or without I₂ treatment. TTGCAAATCTA SEQ ID NO: 12; TTGCATATCTA SEQ ID NO: 13; CTGTAAACTCA SEQ ID NO: 14; CTGTATACTCA SEQ ID NO: 15; TTTCAAATCAA SEQ ID NO: 16; TTTCATATCAA SEQ ID NO: 17; CTTCCAAACA SEQ ID NO: 18. (B) Sequence logo around the anticodon of the four mitochondrial tRNAs. (C) Modification Index (MI) plots of mt-tRNA-Cys-GCA. MI plot is on the left, mutation plot in the middle, and stop plot on the right. (D) MI plots of mt-tRNA-Tyr-GTA. Arrows indicate the nucleotide 3′ next to the anticodons. “−”, without I₂ treatment; “+”, with I₂ treatment.

FIG. 40 shows: Titration experiments for UV-based quantitative analysis of i⁶A. Left: Fluorescence monitoring of i⁶A nucleoside compared to i⁶A nucleoside treated with I₂. Right: Linear correction of fluorescence intensity for i⁶A nucleoside with I₂ treatment (upper panel) and fluorescence intensity of i⁶A nucleoside without I₂ treatment (lower panel).

FIG. 41 shows: Liquid chromatography-tandem mass spectrometry (LC-MS/MS) analysis of the RNA samples. The assays encompass: wild-type RNA samples, knockout samples, and wild-type samples treated with H₂O₂ (12 mM, 1h) from Saccharomyces cerevisiae.

FIGS. 42a-42h SHOW: (A) TBE-Urea gel showing tRNAs isolated from both the WT and KO strains with or without H₂O₂ treatment. The symbol “]” indicates mature tRNAs. (B) Plot showing the mapping proportion of each sample. (C) Pearson correlation coefficient between tRNA-seq samples. (D) Metagene analysis of scaled sequence coverage across cytoplasmic tRNA isotypes. The y-axis values are normalized to the second-to-last bin from the 3′ end. Each x-axis bin represents 4% of the tRNA length. Major known modifications are labeled under the x-axis, including m¹G, m²₂G, m³C, A-I, and m¹A. (E) Mutation rate (MR) plot of cy-tRNA-Ser-AGA-1 in the WT and KO strains, both with or without I₂ treatment. (F) Plot showing the mutation of A37 in 8 i⁶A37-containing tRNAs in the sequencing reads of WT strains after I₂ treatment. (G) Sequences of cy-tRNA-Ser-AGA-1 and cy-tRNA-Ser-AGA-2. The single nucleotide difference between these two tRNAs is highlighted within a dashed box. (H) Mutation rate at position 37 in i⁶A37-containing tRNAs in the WT strain, with or without H₂O₂ (12 mM, 1h) treatment. Statistics calculated by Wilcoxon test. * p<0.05.

FIG. 43. Table 1. Mass results analysis of tagged tRNA.

FIG. 44. Templated EGPF sequence (SEQ ID NO: 9).

FIG. 45 shows mutant results for mRNA incorporating i⁶A (templated with EGPF); SEQ ID NO: 9.

### DETAILED DESCRIPTION

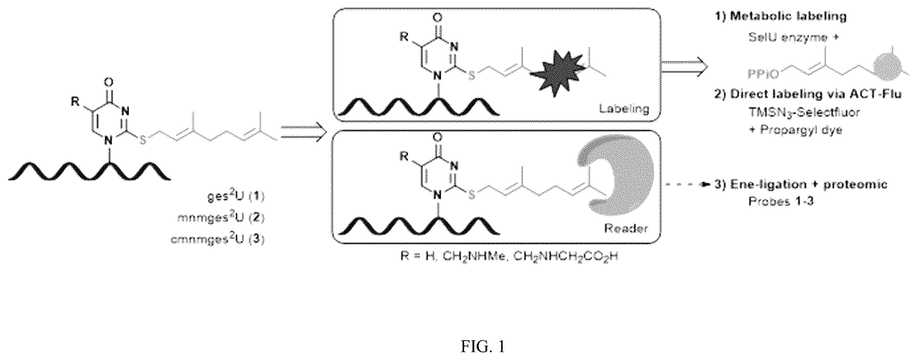

This disclosure relates to a method of labeling lipidated ribonucleosides, and also of identifying lipidated ribonucleosides by cyclization followed by reverse transcription and sequencing of cDNA. In one respect, a significant hydrophobic modification is present on the wobble position of some specific bacterial tRNAs (ges²U, FIG. 1 compound 1, 2 and 3). Disclosed herein are an indirect two-step procedure referred to herein as azidation-and-click tagging of fluorescent dye (ACT-Flu) and a direct metabolic incorporation and biorthogonal tagging (MIBT-Tag). As disclosed herein, the distinct chemical reactivity of prenyl groups on modified RNA can be adapted to investigate lipid modified RNAs, as well as to interrogate their interacting enzymes in living cells. An overview is presented in FIG. 1.

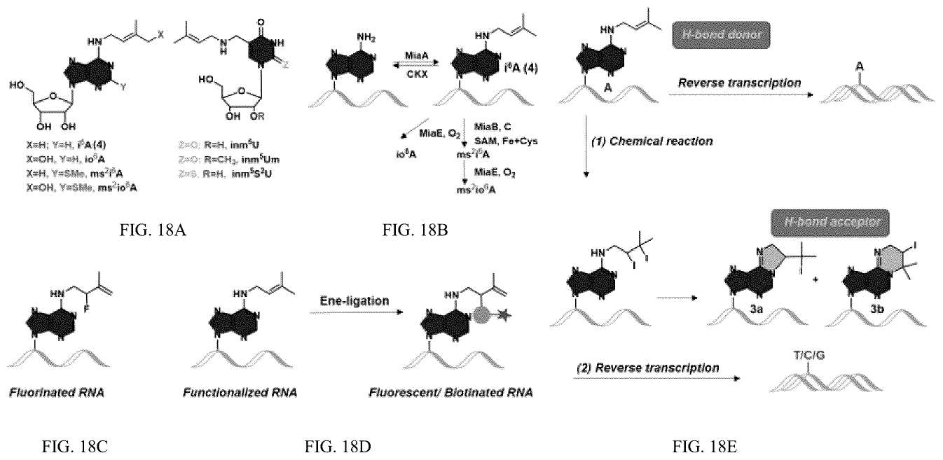

Also disclosed herein is an Ene-ligation strategy for probing chemical properties of a modified ribonucleotide's prenyl group. Hydrophobic prenyl groups (mainly in i⁶A analogs introduced by the Mia family enzyme, FIGS. 18A and 18B) occur in RNAs, such as tRNAs. An Ene-ligation strategy may be used to investigate chemical properties of the prenyl group under biologically mild conditions. This method has may be used in decoding the importance of prenylated RNA residues with the emphasis on the distribution of these modifications in both healthy and diseased cells. This method can also be expanded in investigating the transcriptomic-wide profiling of these lipid-like modifications. As disclosed herein is a mild 2,3-rearrangement Ene-ligation based strategy (FIGS. 18C and 18D) for the fluorescence labeling and detection of prenyl modified RNAs via a σ-rearrangement mechanism. Also disclose herein is a chemical transformation of the prenyl group in reacting with iodine and an iodine-mediated cyclization and reverse transcription (IMCRT) approach to profile the i⁶A residues in cellular RNAs with a single-base resolution (FIG. 18E).

Disclosed are analogues of, for example, geranyl-pyrophosphate (geranyl-OPPi), Ge1:

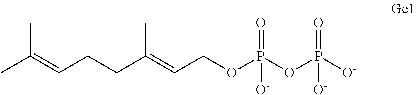 [SMILES: CC(C)=CCC/C(C)=C/COP(=O)([O-])OP(=O)([O-])[O-]]

Compounds include a compound of Formula I:

In an aspect, provided is A compound of Formula I:

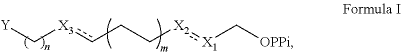

wherein X₁ may be —C(═O)— or —CH₂—, each [symbol] may be, independently, a single bond or a double bond, X₂ may be —NH— or —C(CH₃)—, m may be 0 or 1, X₃ may be —CH₂— or —C(CH₃)—, n may be an integer of from 0 to 8, and Y may be N₃ or

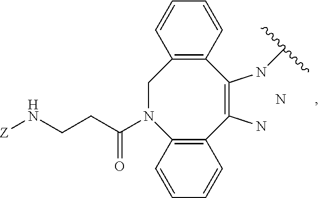

wherein Z may be a fluorescent label. n may be an integer of from 1 to 8. m may be 1. m may be 1 and n may be 8. m may be 0 and n may be 1. Some examples are shown in FIG. 2.

A compound of Formula I may be added to a tRNA ribonucleotide, including a tRNA polyribonucleotide, such as by an enzyme such as SelU. As disclosed herein, inclusion of a reactive moiety for Y in Formula I allows for subsequent addition of a label thereto, where the label includes a chemical group that reacts with the chemical moiety of Y. Chemical moieties and chemical groups that may for covalent attachments to each other via click chemistry reactions may be used, for example addition of an azide group at the Y position of a compound of Formula I, and addition of a cyclooctyne such as dibenzocyclooctyne (DBCO) added to a label. A click chemistry reaction may then be performed to covalently attach the label to the compound of Formula via click chemistry.

A compound of Formula I may be attached to a tRNA during a first reaction, such as in a reaction catalyzed by an enzyme such as SelU, before a label is attached to the compound of Formula I. A second reaction, such as a click chemistry reaction, may then be performed to attach a label activated with a click chemistry appropriate chemical group, resulting in attachment of the label to the tRNA. In another example, a label may be attached at the Y of Formula I (e.g., via a click chemistry reaction) before attachment of the compound of Formula I to an RNA. Subsequent attachment of such a compounds of Formula I to an RNA may result in covalent attachment of the label to the RNA.

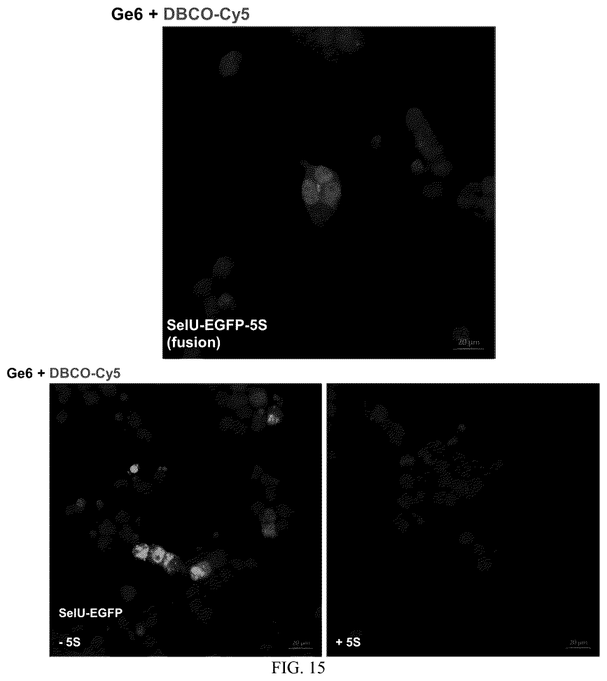

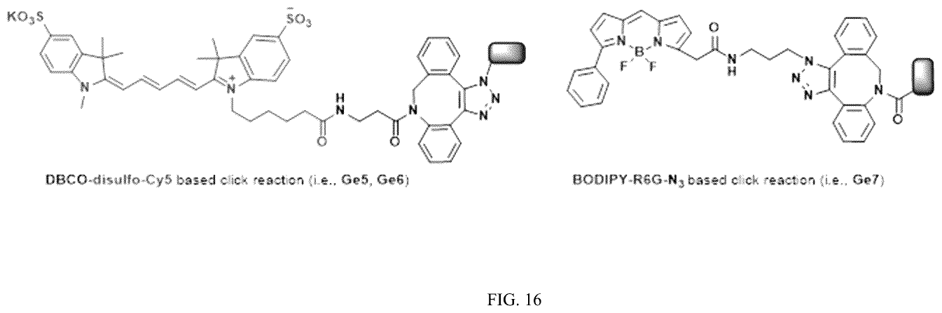

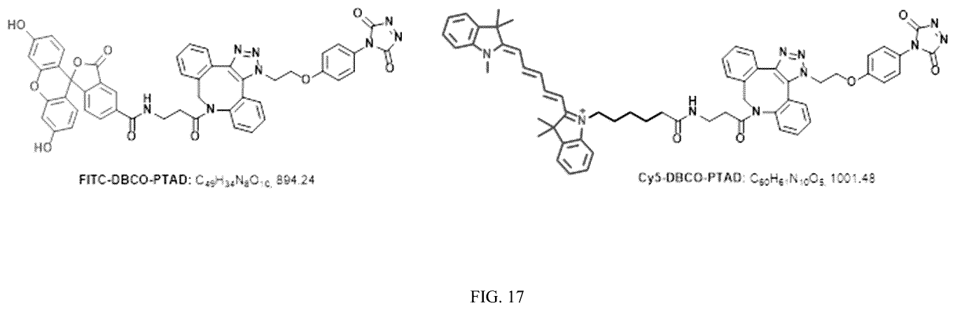

FIG. 16 shows examples of labels, in this case fluorescent labels Cy5 and BODIPY, suitable for attachment to tRNA according to the present disclosure (e.g., via Ge5 or Ge6). Other labels with reactive chemical groups attached may also be used, where they include chemical groups that can react with a reactive group at the Y position of a compound of Formula I. A Formula I-azide-to-cyclooctyne-activated-label is an example of a pairing disclosed herein, but others may be substituted. A list of nonlimiting examples includes amine-NHS, amine-imidoester, amino-pentophenyl ester, amine-hydroxymethyl phosphine, amine-carboxylic acid, thiol-maleimide, thiol-haloacetyl, thiol-pyridyl disulfide, thiol-vinyl sulfone, aldehyde-hydrazide, aldehyde-alkoxyamine, hydroxy-isocyanate, azide-alkyne, azide-phosphine, azide-cyclooctyne, azide-norbornene, transcyclooctene-tetrazine, and norbornene-tetrazine. Inclusion of one of such a pairing at the Y position of a compound of Formula I and the other of such a pairing on a label permits covalent attachment of the label to the compound of Formula I and, upon attachment of the compound of Formula I to a tRNA as disclosed herein, attachment of the label to the tRNA (or attachment of the label to a compound of Formula I attached to a tRNA).

A compound of Formula I as disclosed herein may be Ge5 or Ge6:

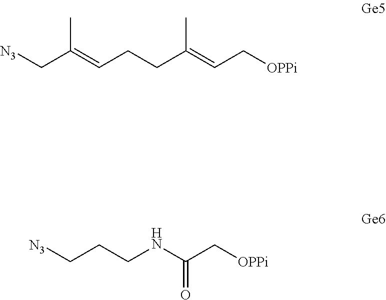

Or, the compound of Formula I may be Ge5 or Ge6 further modified by a click chemistry reaction between the azide of Ge5 or Ge6 with a cyclooctyne of a label, such as a DBCO-activated label. The label may be a fluorescent label. Also disclosed herein is a tRNA with a compound of Formula I, such as Ge5 or Ge6, covalently attached thereto, such as via its 2-thiouridine residue as disclosed herein. Or, in such an example, rather than such an example of a compound of Formula I where the Y position is a moiety for participation in a click chemistry reaction such as an azide as in Ge5 and Ge6, a label may be attached at the Y position of said compound of Formula I, whether by an azide-DBCO connection or other as disclosed above.

A label as disclosed herein may be any chemical structure for identifying, locating, isolating, tagging, or otherwise modifying an RNA. In an example, a label may be a fluorescent label, such as Cy5, FITC, BODIPY, or any other fluorescent composition. Some examples of fluorescent labels include for possible use in a method as disclosed herein include FAM, TAMRA, Cyanine Dye Cy3, Cy3.5, Cy5, Cy5.5, Cy7, Cy7.5, EDANS/Dabcyl, Alexa Fluor, BODIPY FL, and ATTO dyes. Any one of the foregoing fluorescent label may be modified by addition or inclusion of a chemical group and the Y position of a compound of Formula I modified with a complementary moiety whereby the chemical group and the complementary moiety form a bond-forming pair as disclosed above, suitable for attachment of the label to the compound of Formula I and, thus, to a tRNA, e.g. via enzyme (e.g. SelU)-mediated attachment to a 2-thiouridine residue of the tRNA.

Attachment of a label to a compound of Formula I to form a labeled compound of Formula I, or of a compound of Formula I to a tRNA, or a labeled compound of Formula I to a tRNA, or of a tRNA-attached compound of Formula I to a label, or any combination of the foregoing, may be accomplished extracellularly or intracellularly. For intracellular attachment, as disclosed herein, a cell may either intrinsically express an enzyme for enzyme-mediated attachment of a compound of Formula I or labeled compound of Formula I to a 2-thiouridine residue of a tRNA or a transgene may be introduced into the cell to cause the cell to express an enzyme that catalyzes addition of a compound of Formula I or a labeled compound of Formula I to a 2-thiouridine residue of a tRNA, such as SelU as disclosed herein. Or, as disclosed herein, an in vitro method for attaching a compound of Formula I, or labeled compound of Formula I, to a to a 2-thiouridine residue of a tRNA may involve contacting the tRNA and the compound with an enzyme that may catalyze their attachment to each other, such as SelU. Such examples of enzyme-mediated, including SelU-mediated, attachment of a compound of Formula I, or a labeled compound of Formula I, to a 2-thiouridine residue of a tRNA, are a step for covalently attaching a compound of Formula I, or labeled compound of Formula I, to a 2-thiouridine of a tRNA.

Also disclosed is an example of attaching a fluorescent label to a geranylated RNA via Ene ligation. As disclosed herein, direct labelling of prenyl-containing RNAs may be accomplished by such a method. Some tRNAs contains geranyl groups on the wobble position of 34U. As disclosed herein, a 4-phenyl-1,2,4-triazoline-3,5-dione (PTAD) activated fluorescence dye molecule can be directly attached to such terpene terminals through an Ene-reaction, where PTAD reacts with conjugated diene group. For example, tRNA possessing a geranyl group may be fluorescently labeled by contacting it with a PTAD-bound fluorescent label (as a non-limiting example, by way of illustration only, PTAD-DBCO-Cy5). A PTAD-mediated attachment of a label to a geranylated tRNA is a step for covalently attaching a compound of Formula I, or labeled compound of Formula I, to a 2-thiouridine of a tRNA.

Also disclosed herein is synthesis of i⁶A triphosphates (i⁶ATP) and incorporation thereof into RNA strands, and labeling of such compositions. For example, PTAD-activated label such as fluorescent label, as disclosed herein, ay directly bond to an i⁶A moiety of an RNA (e.g., tRNA, mRNA, rRNA, or other RNA). Such PTAD-mediated attachment of a label, including a fluorescent label, to an i⁶A of an RNA strand is a step for performing such a labeling method.

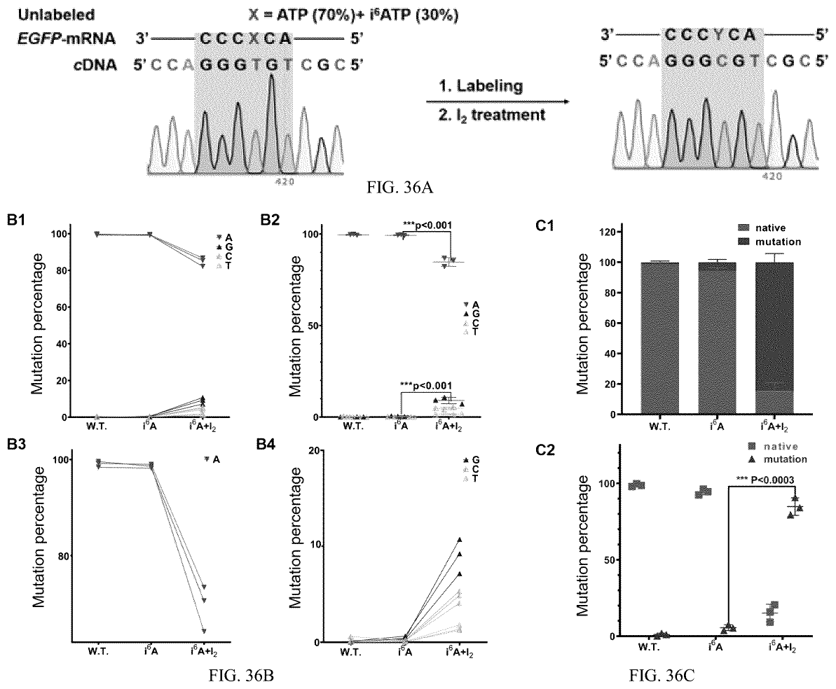

Also disclosed herein is a method of cyclizing i⁶A moieties of RNA by addition of I₂, a reagent for the cyclisation reaction of a prenyl group. Incubating RNA into which i′A has been incorporated (e.g., by an RNA polymerase by adding i⁶A during an RNA polymerization, in living cells or in vitro) then contacting the RNA with I₂ causes a cyclisation of the i⁶A which can subsequently be detected. Adenosine with prenyl modification displays normal base pairing with thymidine. By contrast, the addition of iodine induces the transformation of i⁶A to its cyclic form, thereby diminishing the normal base pairing specificity and resulting in a mixture of complementary G/C/U (FIG. 36A). Reverse transcribing a RNA to a cDNA and sequencing the cDNA may indicate the presence of i⁶A in the RNA (e.g., by comparison to a cDNA sequence reverse transcribed from a comparable RNA sequence that was not contacted by I₂ for cyclisation of i⁶A present therein). Differences in the sequences of the 2 cDNA molecules, resulting from cyclisation of the i⁶A in the RNA exposed thereto before the reverse transcribing, indicates the presence and location of prenylated i⁶A. Such detectable i⁶A incorporation may occur in prokaryotic cells or eukaryotic cells.

### EXAMPLES

The following examples are intended to illustrate particular embodiments of the present disclosure, but are by no means intended to limit the scope thereof.

### Example I: Tagging Lipid-Modified RNAs Via SelU-Mediated tRNA Geranylation Mechanism and Prenyl-Group Reactivity

### Synthesis of Geranyl Pyrophosphate Variants.

### (1) Synthesis of (E)-3, 7-dimethylocta-2, 6-dien-1-yldiphosphate Tetrabutylammonium Trimer (Ge1)

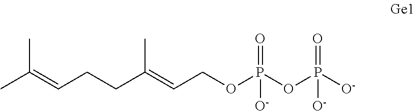 [SMILES: CC(C)=CCC/C(C)=C/COP(=O)([O-])OP(=O)([O-])[O-]]

To a stirred solution containing (E)-1-bromo-3,7-dimethylocta-2,6-diene (50.0 mg, 0.23 mmol) dissolved in acetonitrile (1.5 ml), tris (tetra-N-butylammonium) hydrogen pyrophosphate (200 mg, 0.48 mmol) was carefully and gradually introduced. This reaction mixture was maintained under a nitrogen atmosphere and stirred at room temperature for a period of 5 hours. Upon completion of the reaction, the crude product was subjected to purification through high-performance liquid chromatography (HPLC). Subsequently, the purified product was freeze-dried to obtain a dry solid, yielding a total of 4.3 mg with a percent yield of 6%.

White solid. ¹H NMR (400 MHz, D₂O) δ 5.37 (t, J=6.8 Hz, 1H), 5.11 (t, J=6.7 Hz, 1H), 4.38 (t, J=6.4 Hz, 2H), 2.06 (d, J=7.4 Hz, 2H), 2.01 (d, J=6.7 Hz, 2H), 1.60 (d, J=4.4 Hz, 6H). ³¹P NMR (162 MHz, D₂O) δ −6.85 (d, J=22.68 Hz), −10.57 (d, J=22.68 Hz). MS (ESI): Calculated [M+Na]⁺=337.2. Found [M+Na]⁺=337.0.

### (2) Synthesis of 3, 7-dimethyloct-6-en-1-yl pyrophosphate (Ge2)

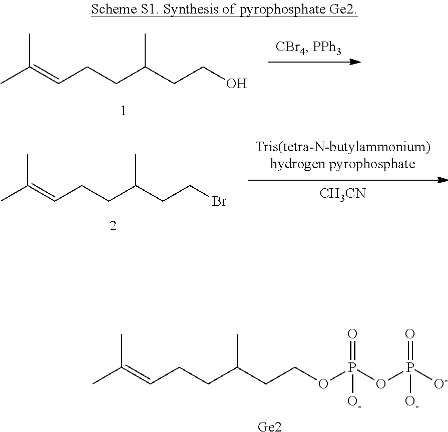

In a well-stirred solution, 3,7-dimethyloct-6-en-1-ol (150 mg, 0.96 mmol) was mixed with carbon tetrabromide (414 mg, 1.25 mmol) and triphenylphosphine (387 mg, equal to 1.47 mmol) in a volume of 3.0 ml of dichloromethane. This reaction mixture was then stirred at a controlled temperature of 0 degrees overnight under an inert nitrogen atmosphere. The crude product was purified using silica gel flash chromatography, which yielded 8-bromo-2,6-dimethyloct-2-ene as a yellow oil (150 mg, 0.69 mmol, 71% yield).

Subsequently, to a fresh solution containing 8-bromo-2,6-dimethyloct-2-ene (80.0 mg, 0.37 mmol), a solution of tris (tetra-N-butylammonium) hydrogen pyrophosphate (423 mg, 1.01 mmol) in 1.5 ml of acetonitrile (CH₃CN) was gently added. This new mixture was again stirred for a duration of 5 hours at room temperature, maintaining an inert nitrogen atmosphere throughout. Upon completion, the resulting crude product was further purified using high-performance liquid chromatography (HPLC). Following purification, the product was lyophilized to obtain a dry solid, with a final yield of 8.2 mg and a percentage yield of 7%.

¹H NMR (400 MHz, CDCl₃) δ 5.13-5.02 (m, 1H), 4.10 (q, J=7.1 Hz, 1H), 3.52-3.30 (m, 2H), 2.01-1.91 (m, 2H), 1.95-1.83 (m, 2H), 1.67 (s, 3H), 1.59 (s, 3H).

Compound Ge2: ¹H NMR (400 MHz, D₂O) δ 5.21 (t, J=7.2 Hz, 2H), 4.02-3.85 (m, 4H), 2.01-1.96 (m, 3H), 1.65 (s, 6H), 1.47-1.38 (m, 3H), 1.15 (dd, J=14.7, 6.6 Hz, 2H), 1.06 (t, J=7.5 Hz, 1H), 0.85 (m, 3H). ³¹P NMR (162 MHz, D₂O) δ −8.45 (d, J=17.3 Hz), −10.53 (d, J=21.06 Hz). MS (ESI): Calculated [M+H]⁺=336.2. Found [M+H]⁺=336.3.

### (3) Synthesis of 4-methylpent-3-en-1-yl Diphosphate Tetrabutyl Ammonium Trimer (Ge3)

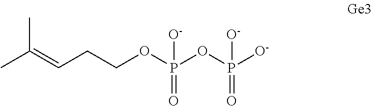

A stirred solution containing 5-bromo-2-methylpent-2-ene (50.0 mg, 0.31 mmol) was gradually treated with a solution of tris (tetra-N-butylammonium) hydrogen pyrophosphate (331 mg, 0.79 mmol) dissolved in 1.5 ml of acetonitrile (CH₃CN). The combined mixture was then stirred for a period of 5 hours at room temperature under an inert nitrogen atmosphere. Upon completion of the stirring period, the resultant crude product was purified using high-performance liquid chromatography (HPLC). Following purification, the product was subjected to lyophilization to achieve a dry state, yielding 4.8 mg with a 6% yield.

White solid. ¹H NMR (400 MHz, D₂O) δ 5.17 (s, 1H), 3.81 (s, 2H), 2.27 (s, 2H), 1.84 (d, J=101.2 Hz, 6H). ³¹P NMR (162 MHz, D₂O) δ −6.52 (d, J=22.68 Hz), −10.59 (d, J=21.06). MS (ESI): Calculated [M+H]⁺=536.4. Found [M+H]⁺=536.8.

### 4. Synthesis of Pyrophosphate Ge4

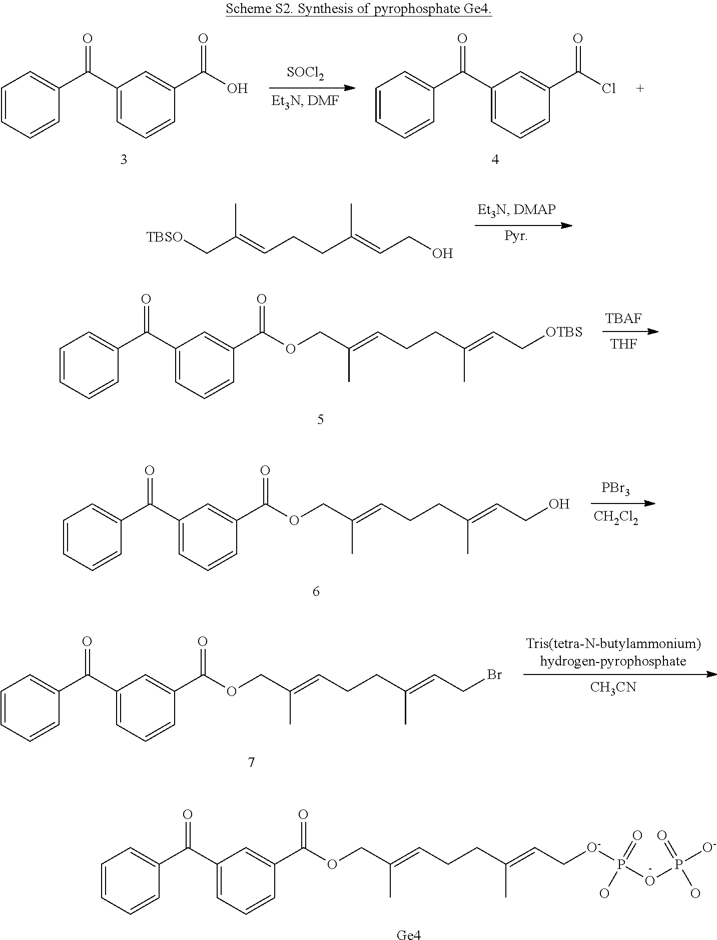

3-Benzoylbenzoic acid (450 mg, 1.99 mmol) was dissolved in a mixture of dichloromethane (4.4 ml) along with a small quantity of dimethylformamide. To this solution, thionyl chloride (SOCl₂, 0.3 ml, 3.97 mmol) was added, and the resulting mixture was continuously stirred at a temperature maintained at reflux for an overnight duration. After the reaction had completed, it was sequentially washed with a 0.1 M sodium hydroxide solution and subsequently extracted using dichloromethane. The organic layer was then dried over anhydrous sodium sulfate. Next, the solvents were removed via evaporation, and the residue obtained was immediately utilized for the subsequent step without further purification or isolation.

Approximately 200 mg of compound 4 (0.82 mmol) was combined with (2E, 6E)-8-((tert-butyl dimethylsilyl)oxy)-3,7-dimethylocta-2,6-dien-1-ol (125 mg, 0.44 mmol) in a reaction mixture that also included triethylamine (Et₃N, 0.2 ml, 1.44 mmol) and 4-dimethylaminopyridine (DMAP, 4.0 mg, 0.033 mmol) dissolved in 2.0 ml of pyridine. This mixture was then stirred consistently for a duration of 5 hours at a temperature of 65 degrees. The reaction was subsequently terminated by the addition of water, followed by a washing step with brine and extraction using dichloromethane. The organic phase was then dried over anhydrous sodium sulfate. Next, the crude product underwent purification through silica-gel flash chromatography, ultimately yielding compound 5 as a white solid (101.9 mg, 47% yield).

Compound 5 (230 mg, 0.47 mmol) and TBAF (600 μl, 0.6 mmol) were combined in 1.5 mL THF and stirred under a nitrogen atmosphere at 30° C. overnight. The reaction was halted using saturated NH₄Cl. After removing THE, the residue was extracted with ethyl acetate and then dried over Na₂SO₄. Purification by silica gel flash chromatography resulted in compound 6 with a yield of 159.8 mg (90%).

At 0° C., to a solution of compound 6 (159.8 mg, 0.42 mmol) in 2.0 ml of dichloromethane, PBr₃ (42.5 μl, 0.44 mmol) was cautiously added. The reaction mixture was stirred overnight at room temperature. Upon completion, it was quenched with water, washed with sodium bicarbonate and brine, then extracted with dichloromethane and dried over anhydrous Na₂SO₄. The crude product was purified by silica gel flash chromatography, yielding compound 7 (180.0 mg, 0.41 mmol, 97% yield).

Under N₂ at 0° C., 0.41 mmol of compound 7 (180.0 mg) dissolved in 0.5 mL CH₃CN was added dropwise to a solution of 0.86 mmol tris (tetra-N-butylammonium) hydrogen pyrophosphate (360.0 mg) in 0.5 mL CH₃CN. The reaction was stirred at room temperature for 3 hours and then purified by HPLC, yielding a white solid (10.9 mg, 5% yield).

Compound 5: ¹H NMR (400 MHz, CDCl₃) δ 8.44 (d, J=7.2 Hz, 1H), 8.26 (t, J=5.2 Hz, 1H), 8.02-7.96 (m, 1H), 7.80 (d, J=8.9 Hz, 2H), 7.61 (t, J=8.7 Hz, 1H), 7.56 (d, J=7.7 Hz, 1H), 7.50 (t, J=7.6 Hz, 2H), 5.47 (t, J=7.0 Hz, 1H), 5.37 (t, J=6.7 Hz, 1H), 4.86 (d, J=7.1 Hz, 2H), 3.99 (s, 2H), 2.21-2.13 (m, 2H), 2.14-2.07 (m, 2H), 1.59 (s, 5H), 0.89 (s, 9H), 0.05 (s, 6H). ¹³C NMR {¹H} (101 MHz, CDCl₃) δ 165.85 (s), 142.57 (s), 137.93 (s), 137.08 (s), 134.84 (s), 133.97 (s), 133.22 (s), 132.80 (s), 131.00 (s), 128.47 (s), 123.58 (s), 118.23 (s), 68.50 (s), 39.26 (s), 25.96 (s), 18.43 (s), 16.59 (s), 13.47 (s), 0.01 (s).

Compound 6: ¹H NMR (400 MHz, CDCl₃) δ 8.24 (d, J=7.9 Hz, 2H), 7.96 (t, J=6.5 Hz, 2H), 7.48 (t, J=7.6 Hz, 5H), 5.50 (dd, J=15.2, 8.3 Hz, 1H), 5.44 (d, J=7.0 Hz, 1H), 4.83 (d, J=7.1 Hz, 2H), 4.69 (s, 2H), 2.24-2.16 (m, 2H), 2.15-2.06 (m, 2H), 1.75 (s, 2H), 1.70 (s, 3H). ¹³C NMR {¹H} (101 MHz, CDCl₃) δ 196.37 (s), 166.36 (s), 142.63 (s), 138.46 (s), 137.56 (s), 134.56 (d, J=4.0 Hz), 133.73 (s), 133.35 (s), 131.52 (d, J=5.1 Hz), 130.86 (s), 130.60 (s), 129.68 (s), 129.23-128.90 (m), 119.10 (s), 71.43 (s), 62.70 (s), 39.41 (s), 30.23 (s), 17.13 (s), 14.63 (s).

Compound 7: ¹H NMR (400 MHz, CDCl₃) δ 8.43 (d, J=1.5 Hz, 2H), 8.25 (d, J=7.7 Hz, 2H), 7.80 (d, J=7.0 Hz, 5H), 5.22 (dd, J=17.3, 1.0 Hz, 1H), 5.10-5.02 (m, 1H), 4.38 (d, J=6.0 Hz, 2H), 3.95 (s, 2H), 2.15-2.08 (m, 2H), 2.07-2.02 (m, 2H), 1.74 (d, J=5.5 Hz, 6H). ¹³C NMR {¹H} (101 MHz, CDCl₃) δ 195.98 (s), 166.03 (s), 138.14 (s), 137.23 (s), 134.22 (s), 133.34 (s), 133.02 (s), 132.27 (s), 131.52 (s), 131.15 (s), 130.93 (s), 130.34 (d, J=15.1 Hz), 129.69 (s), 128.72 (d, J=9.6 Hz), 121.33 (s), 68.73 (s), 63.97 (s), 36.31 (s), 35.60 (s), 29.81 (s), 25.90 (s), 19.60 (s), 14.84 (s).

White solid. Pyrophosphate Ge3: ¹H NMR (400 MHz, D₂O) δ 8.32 (d, J=6.5 Hz, 4H), 8.11 (d, J=8.1 Hz, 2H), 7.83 (d, J=7.6 Hz, 5H), 7.76 (t, J=8.1 Hz, 2H), 7.65 (t, J=7.7 Hz, 4H), 5.61 (t, J=5.8 Hz, 2H), 4.44 (s, 2H), 4.36 (d, J=5.8 Hz, 3H), 2.13-2.07 (m, 2H), 1.89-1.82 (m, 2H), 1.72 (s, 6H). ³¹P NMR (162 MHz, D₂O) δ −9.71 (d, J=26.3 Hz), −10.86 (d, J=24.3 Hz). MS (ESI): Calculated [M+H]⁺=536.4. Found [M+H]⁺=536.8.

### 5. Synthesis of Pyrophosphate Ge5

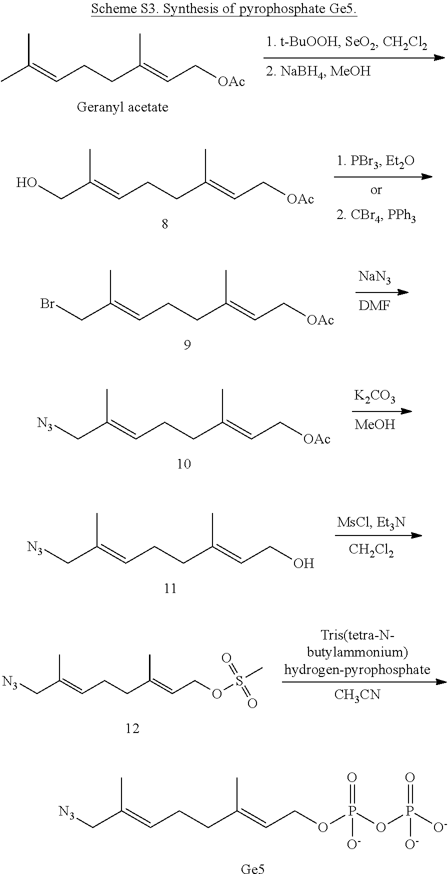

(E)-3,7-dimethylocta-2,6-dien-1-ylacetate (201.0 mg, 1.03 mmol) was reacted with t-BuOOH (122 μL, 2.06 mmol) and SeO₂ (17.1 mg, 0.15 mmol) in 2.0 ml dichloromethane at room temperature for 3 days. The cooled mixture to 0° C. was then treated with NaBH₄ (total 600 mg in six portions) until defoaming ceased after 5 hours stirring. The reaction was washed with water, extracted with dichloromethane, and dried over Na₂SO₄. Purification via silica gel flash chromatography gave compound 8 (120 mg, 0.57 mmol, 55% yield).

Subsequently, compound 8 (120 mg, 0.57 mmol) was then reacted with PBr₃ (1.31 g, 4.83 mmol) in 0.8 mL diethyl ether for 4 hours at an ice bath under a nitrogen atmosphere. Further purification of the crude residue through silica gel flash chromatography led to the isolation of the desired product 9 (141.2 mg, 0.51 mmol, 90% yield).

Compound 9 (50.0 mg, 0.18 mmol) and NaN₃ (23.0 mg, 0.35 mmol) were dissolved in 1.00 mL DMF and stirred under reflux at 75° C. overnight. Silica gel flash chromatography purified the reaction mixture, yielding compound 10 (40.0 mg, 0.17 mmol) with a 94% yield.

To a stirred solution of compound 10 (40.0 mg, 0.17 mmol) in methanol (2.0 ml), potassium carbonate (58.0 mg, 0.42 mmol) was added and stirred for 3 hours at room temperature. The mixture was then washed with deionized water, extracted with ethyl acetate, and dried over anhydrous sodium sulfate. This crude product was transferred into anhydrous methanol (5.0 ml), the reaction was stirred overnight at room temperature under a nitrogen atmosphere. After treating with saturated sodium bicarbonate and further drying over anhydrous Na₂SO₄, the crude product was purified through silica gel flash chromatography with a 10% ethyl acetate in petroleum ether (PE) eluent, affording compound 11 (33.0 mg, 0.17 mmol, 100% yield).

Compound 11 (33.0 mg, 0.17 mmol) was dissolved in 1.0 ml dichloromethane, followed by the addition of MsCl (20.0 μL, 0.26 mmol) and Et₃N (40.0 μL, 0.29 mmol) at 0° C. The mixture was stirred overnight and then vacuum-evaporated. The residue was purified by silica gel flash chromatography to give 12, which was directly used for the subsequent step without further isolation.

Compound 12 (0.17 mmol) was dissolved in a stirred solution, then tris (tetra-N-butylammonium) hydrogen pyrophosphate (220 mg, 0.53 mmol) in 1.0 ml CH₃CN was slowly added. The reaction was stirred for 5 hours at room temperature under N₂. The crude product was purified by HPLC and lyophilized, yielding a white solid (3.0 mg, 5% yield).

Alcohol 8: ¹H NMR (400 MHz, CDCl₃) δ 5.35 (m, 2H), 4.57 (s, 2H), 3.97 (s, 2H), 2.16 (dd, J=14.6, 7.0 Hz, 2H), 2.07 (t, J=5.9 Hz, 2H), 2.04 (s, 3H), 2.03 (s, 2H), 1.65 (s, 4H). ¹³C NMR {¹H} (101 MHz, CDCl₃) δ 171.21 (s), 141.73 (s), 135.22 (s), 124.74 (s), 118.47 (s), 68.31 (s), 61.33 (s), 39.01 (s), 25.63 (s), 20.88 (s), 16.21 (s), 13.54 (s). HRMS (ESI): Calculated [M+Na]=235.1310. Found [M+Na]=235.1308.

Bromide 9: ¹H NMR (400 MHz, CDCl₃) δ 5.56 (dd, J=12.3, 6.2 Hz, 1H), 5.33 (t, J=7.6 Hz, 1H), 4.57 (d, J=7.1 Hz, 2H), 3.95 (s, 2H), 2.18-2.08 (m, 7H), 1.74 (s, 3H), 1.69 (s, 3H). ¹³C NMR {¹H} (101 MHz, CDCl₃) δ 171.28 (s), 141.50 (s), 132.66 (s), 130.65 (s), 119.08 (s), 61.48 (s), 41.74 (s), 38.75 (s), 26.60 (s), 21.24 (s), 16.63 (s), 14.87 (s).

Azide 10: ¹H NMR (400 MHz, CDCl₃) δ 5.46-5.40 (m, 1H), 5.38 (m, 1H), 4.96 (dd, J=2.6, 1.3 Hz, 2H), 4.56 (d, J=7.1 Hz, 2H), 3.77 (dd, J=7.5, 5.4 Hz, 2H), 2.32-2.20 (m, 7H), 1.70 (d, J=0.8 Hz, 6H). ¹³C NMR {¹H} (101 MHz, CDCl₃) δ 171.17 (s), 142.28 (s), 130.46 (s), 129.56 (s), 114.61 (d, J=14.6 Hz), 68.80 (d, J=6.7 Hz), 62.86 (s), 59.41 (s), 33.15 (s), 25.20 (s), 20.99 (s).

Compound 11: ¹H NMR (400 MHz, CDCl₃) δ 5.50-5.43 (m, 1H), 5.42 (t, J=6.6 Hz, 2H), 4.98 (dd, J=3.0, 1.5 Hz, 2H), 3.81 (d, J=5.8 Hz, 2H), 2.05 (td, J=14.8, 6.9 Hz, 4H), 1.72 (d, J=0.8 Hz, 6H). ¹³C NMR {¹H} (101 MHz, CDCl₃) δ 131.25 (s), 129.97 (s), 115.13 (d, J=16.3 Hz), 69.35 (s), 61.61 (s), 59.99 (s), 40.33 (s), 37.28 (s), 33.87 (s), 30.23 (s), 29.62 (s), 25.81 (s), 19.96 (s), 18.04 (s), 15.14 (s).

White solid. Pyrophosphate Ge5: ¹H NMR (400 MHz, D₂O) δ 5.52-5.48 (m, 2H), 3.60-3.56 (m, 2H), 2.17 (m, 5.3 Hz, 2H), 2.11-2.04 (m, 2H), 1.68 (s, 3H), 1.65 (s, 3H). ³¹P NMR (162 MHz, D₂O) δ −6.64 (d, J=22.7 Hz), −10.44 (d, J=22.7 Hz). MS (ESI): Calculated [M+H]⁺=375.2. Found [M+H]⁺=375.5.

### 6. Synthesis of Pyrophosphate Ge6

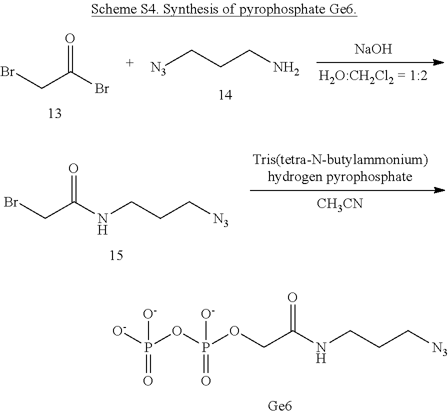

2-Bromoacetyl bromide (13, 0.696 mL, 8.0 mmol), 3-azidopropan-1-amine (14, 100 mg, 1.0 mmol), and NaOH (200 mg, 5.0 mmol) were combined in a CH₂Cl₂/H₂O (2.5 mL: 1.2 mL) solvent system and stirred overnight at room temperature. The aqueous layer was extracted with CH₂Cl₂, washed with 50 mM Na₂CO₃, dried over Na₂SO₄, and purified via silica gel flash chromatography, yielding compound 15 (221.1 mg, 1.0 mmol, 100% yield).

Compound 15 (50.0 mg, 0.23 mmol) was added slowly to a stirred solution containing tris (tetra-N-butylammonium) hydrogen pyrophosphate (220.1 mg, 0.53 mmol) in 1.0 mL of CH₃CN. The mixture was stirred for 5 hours at room temperature under a nitrogen atmosphere. The crude product was purified by HPLC, lyophilized, and obtained as a white solid (5.1 mg, 7% yield).

Compound 15: ¹H NMR (400 MHz, CDCl₃) δ 4.22 (t, J=6.0 Hz, 2H), 4.12 (t, J=14.3, 7.3 Hz, 3H), 2.46-2.36 (m, 2H), 2.33-2.25 (m, 2H).

White solid. Pyrophosphate Ge6: ¹H NMR (400 MHz, D₂O) δ 4.88 (d, J=6.4 Hz, 2H), 3.50-3.29 (m, 2H), 1.17 (dd, J=14.9, 7.4 Hz, 2H), 1.07 (t, J=7.4 Hz, 2H). 31P NMR (162 MHz, D₂O) δ −5.87 (d, J=22.7 Hz), −11.19 (d, J=22.3 Hz). MS (ESI): Calculated [M+H]⁺=316.1. Found [M+H]⁺=316.5.

### 7. Synthesis of Pyrophosphate Ge7

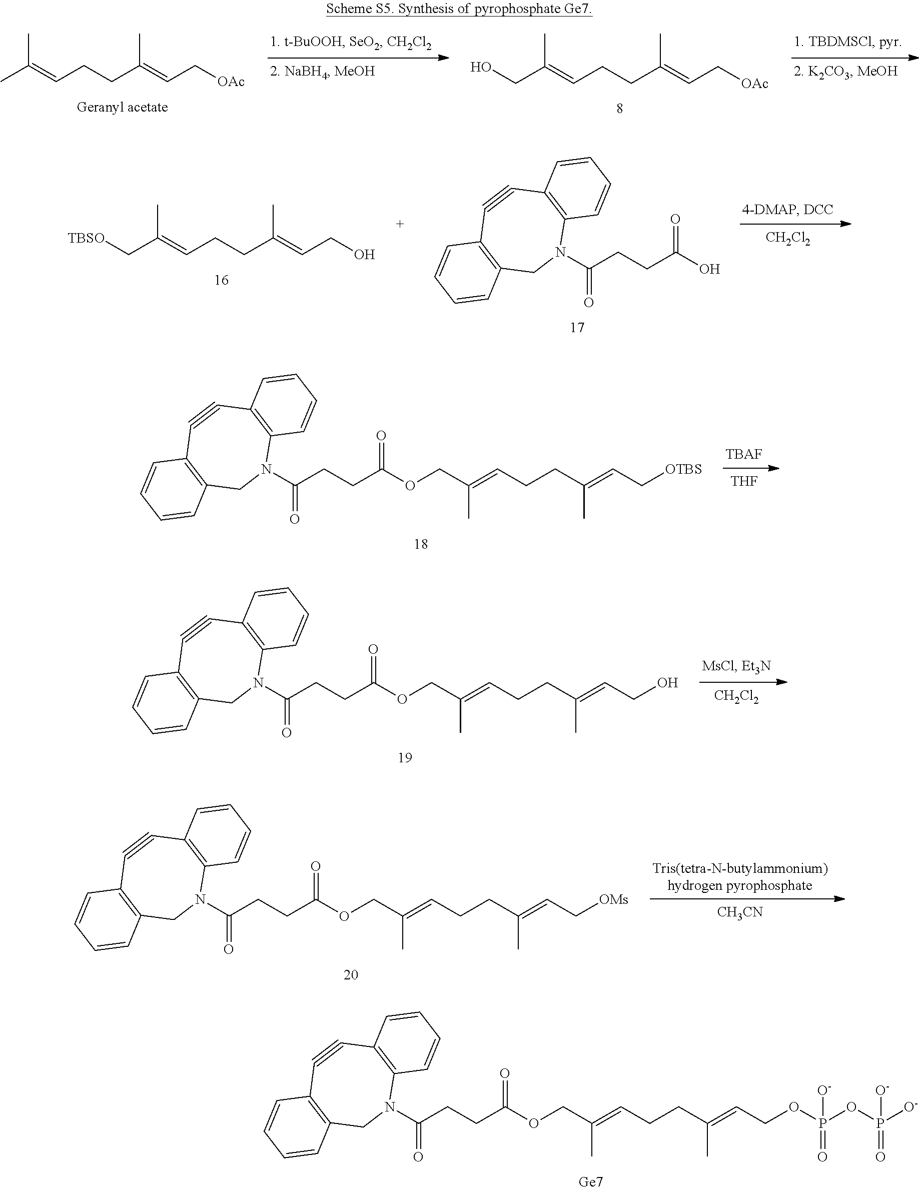

Compound 8 (500 mg, 2.36 mmol) in anhydrous pyridine (6.0 ml) was treated with tert-butyl chlorodimethylsilane (423 mg, 2.81 mmol) under N₂ at 0° C. for 2 hours. Next, solvents were evaporated under vacuum, and the residue was dissolved in CH₂Cl₂. The crude product was purified via silica gel flash chromatography using a petroleum ether-ethyl acetate (14:1) eluent, yielding compound 16 (600 mg, 2.11 mmol) with an 89% yield.

To a stirred dichloromethane (1.5 ml) solution, compound 16 (60.0 mg, 0.21 mmol), 17 (50.0 mg, 0.16 mmol), dicyclohexylcarbodiimide (80.0 mg, 0.39 mmol), and N-(4-pyridyl)-dimethylamine (10.0 mg, 0.082 mmol) were added, then the mixture was stirred overnight at room temperature under N₂. Upon reaction completion, pH was adjusted to 5-6 using 0.1 M HCl, followed by washing with saturated NaHCO₃ and saturated aqueous NaCl, extracting with dichloromethane, and drying over Na₂SO₄. The crude product was concentrated in a rotary evaporator and purified by silica gel flash chromatography with a gradient eluent of petroleum ether-ethyl acetate (from 60:1 to 30:1), yielding compound 18 (58.6 mg, 0.10 mmol) with a 64% yield.

To a stirred tetrahydrofuran (1.0 ml) solution containing compound 18 (60.0 mg, 0.10 mmol), tetrabutylammonium fluoride (0.4 ml, 0.40 mmol) was added. The mixture was stirred overnight at room temperature under a nitrogen atmosphere. Upon completion, the reaction was quenched with saturated NH₄Cl and concentrated in a rotary evaporator. The crude product was extracted with CH₂Cl₂, dried over Na₂SO₄, and further concentrated. It was then purified via silica gel flash chromatography using a gradient elution of petroleum ether-ethyl acetate (30:1 to 10:1) to afford alcohol 19 (38.1 mg, 0.083 mmol) with an 83% yield.

Alcohol 19 (38 mg, 0.083 mmol) was dissolved in 1.0 ml dichloromethane and cooled to 0° C. MsCl (20.0 μl, 0.26 mmol) and Et₃N (40.0 μl, 0.29 mmol) were then added. The mixture was stirred overnight and vacuum-evaporated to yield compound 20, which was directly employed in the subsequent step without further purification.

To a stirred solution of compound 20, tris (tetra-N-butylammonium) hydrogen pyrophosphate (101.1 mg, 0.24 mmol) in 1.0 ml CH₃CN was added slowly. The mixture was stirred for 5 hours at room temperature under a nitrogen atmosphere. The crude product was purified by HPLC, lyophilized, and obtained as a white solid with a yield of 5% (2.5 mg).

Compound 18: ¹H NMR (400 MHz, CDCl₃) δ 7.68 (d, J=7.6 Hz, 1H), 7.51 (dd, J=5.7, 3.2 Hz, 1H), 7.40 (dd, J=8.9, 5.1 Hz, 2H), 7.37 (d, J=1.4 Hz, 1H), 7.35 (d, J=1.6 Hz, 1H), 7.35-7.32 (m, 1H), 5.34 (t, J=6.8 Hz, 1H), 5.26 (t, J=7.6 Hz, 1H), 5.16 (d, J=13.9 Hz, 1H), 3.99 (s, 2H), 3.66 (d, J=8.3 Hz, 1H), 2.74 (ddd, J=16.2, 8.3, 6.4 Hz, 1H), 2.63 (ddd, J=17.1, 8.3, 6.3 Hz, 1H), 2.31 (dt, J=17.2, 6.1 Hz, 1H), 2.15-2.09 (m, 2H), 1.95 (dd, J=10.2, 6.2 Hz, 2H), 1.92 (d, J=6.0 Hz, 2H), 1.73-1.69 (m, 2H), 1.67 (d, J=4.8 Hz, 2H), 1.65 (s, 3H), 0.89 (s, 8H), 0.04 (s, 6H).

Alcohol 19: ¹H NMR (400 MHz, CD₃OD) δ 7.54 (d, J=8.1 Hz, 1H), 7.52-7.47 (m, 1H), 7.36 (d, J=2.2 Hz, 2H), 7.29-7.19 (m, 2H), 7.15 (d, J=7.2 Hz, 1H), 7.12 (d, J=2.4 Hz, 1H), 5.25 (dd, J=10.0, 7.1 Hz, 2H), 3.80 (s, 2H), 3.66-3.56 (m, 1H), 2.41-2.17 (m, 4H), 2.00-1.88 (m, 4H), 1.86 (s, 3H), 1.57 (s, 3H), 1.54 (s, 3H).

White solid. Pyrophosphate Ge7: ¹H NMR (400 MHz, CDCl₃) δ 7.69 (dd, J=17.0, 8.2 Hz, 4H), 7.61-7.56 (m, 4H), 7.37 (s, 2H), 4.30 (d, J=1.5 Hz, 6H), 4.10-4.08 (m, 2H), 3.75 (s, 2H), 3.62 (s, 2H), 2.20-2.08 (m, 4H), 2.04 (s, 3H), 1.97 (s, 1H), 1.79 (s, 3H), 1.76 (s, 3H). ³¹P NMR (162 MHz, D₂O) δ −6.61 (d, J=22.0 Hz), −10.74 (d, J=19.5 Hz). MS (ESI): Calculated [M−H]⁺=613.5. Found [M−H]⁺=613.0.

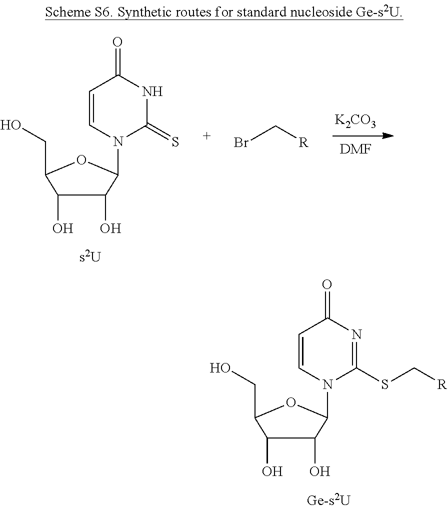

### (1) Synthesis of 1-((2R, 3R, 4S, 5R)-3, 4-dihydroxy-5-(hydroxymethyl) tetrahydrofuran-2-yl)-2-(((E)-3, 7-dimethylocta-2, 6-dien-1-yl)-thio)-pyrimidin-4 (1H)-one (22)

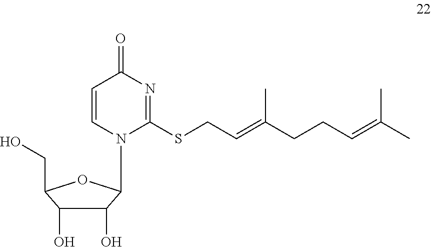 [SMILES: CC(C)=CCC/C(C)=C/CSc1nc(=O)ccn1C1OC(CO)C(O)C1O]

The mixture solution of nucleoside 21 (s²U, 5.0 mg, 0.019 mmol), geranyl bromide (13.3 mg, 0.057 mmol) and DIPEA (7.0 mg, 0.057 mmol) in MeOH (0.5 mL) was stirred overnight at r.t. under N₂ atmosphere. The crude residue was purified by preparative thin layer chromatography and confirmed by ¹H NMR and MS (ESI).

¹H NMR (400 MHz, CDCl₃) δ 8.23 (d, J=8.0 Hz, 1H), 6.05 (s, 1H), 6.03 (s, 1H), 5.87 (d, J=3.3 Hz, 1H), 5.58-5.57 (m, 1H), 5.51 (s, 1H), 5.06 (s, 1H), 4.86 (s, 1H), 4.37 (s, 1H), 4.07 (s, 1H), 4.06 (s, 1H), 2.00-1.99 (m, 2H), 1.99-1.97 (m, 2H), 1.75 (s, 3H), 1.74 (s, 3H). MS (ESI): Calculated [M+H]⁺=397.5. Found [M+H]⁺=398.2.

### (2) Synthesis of 1-((2R, 3R, 4S, 5R)-3, 4-dihydroxy-5-(hydroxymethyl)-tetrahydrofuran-2-yl)-2-((4-methylpent-3-en-1-yl)-thio)-pyrimidin-4-(1H)-one (23)

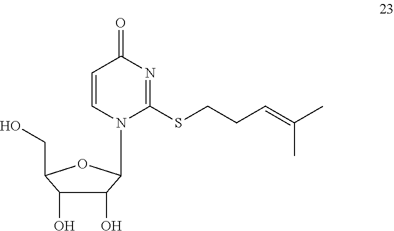 [SMILES: CC(C)=CCCSc1nc(=O)ccn1C1OC(CO)C(O)C1O]

The mixture solution of nucleoside 21 (s²U, 5.0 mg, 0.0190 mmol), 5-bromo-2-methylpent-2-ene (13.3 mg, 0.0570 mmol) and DIPEA (7.0 mg, 0.057 mmol) in MeOH (0.5 mL) was stirred overnight at r.t. under N₂ atmosphere. The residue was purified by preparative thin layer chromatography and confirmed by ¹H NMR and MS (ESI).

¹H NMR (400 MHz, CDCl₃) δ 8.18 (s, 1H), 6.19 (d, J=5.3 Hz, 1H), 4.47-4.42 (m, 2H), 4.40 (t, J=3.6 Hz, 1H), 4.36 (d, J=4.2 Hz, 1H), 4.32 (d, J=6.6 Hz, 1H), 4.13-4.01 (m, 2H), 3.43 (dd, J=15.2, 11.8 Hz, 2H), 2.94-2.83 (m, 2H), 1.68 (d, J=16.1 Hz, 6H). MS (ESI): Calculated [M+Na]⁺=365.4. Found [M+Na]⁺=365.3.

### (3) Synthesis of N-(3-azidopropyl)-2-((1-((2R, 3R, 4S, 5R)-3,4-dihydroxy-5-(hydroxymethyl)-tetrahydrofuran-2-yl)-4-oxo-1, 4-dihydropyrimidin-2-yl)-thio)acetamide (24)

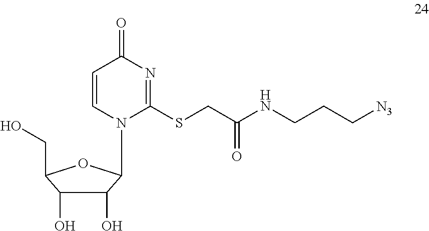 [SMILES: [N-]=[N+]=NCCCNC(=O)CSc1nc(=O)ccn1C1OC(CO)C(O)C1O]

To a stirred solution of nucleoside 21 (s²U, 5.0 mg, 0.0190 mmol), 5-bromo-2-methylpent-2-ene (13.3 mg, 0.0570 mmol) and DIPEA (7.00 mg, 0.057 mmol) in MeOH (0.5 mL). The mixture was stirred overnight at r.t. under N₂ atmosphere. The residue was purified by preparative thin layer chromatography and confirmed by ¹H NMR and MS (ESI).

¹H NMR (400 MHz, CDCl₃) δ 8.07 (d, J=6.9 Hz, 1H), 5.78 (d, J=3.4 Hz, 1H), 5.33 (s, 1H), 4.87 (dd, J=12.9, 2.4 Hz, 1H), 4.77 (d, J=2.7 Hz, 1H), 4.67 (dd, J=12.5, 2.9 Hz, 1H), 3.69-3.55 (m, 1H), 2.20 (dd, J=11.9, 5.1 Hz, 1H), 2.02-1.97 (m, 1H), 1.40 (d, J=4.8 Hz, 2H), 1.30 (d, J=5.3 Hz, 2H). MS (ESI): Calculated [M+Na]⁺=423.4. Found [M+Na]⁺=423.0.

### (4) Synthesis of 1-((2R, 3R, 4S, 5R)-3, 4-dihydroxy-5-(hydroxymethyl)-tetrahydrofuran-2-yl)-2-((3, 7-dimethyloct-6-en-1-yl)thio)-pyrimidin-4(1H)-one (25)

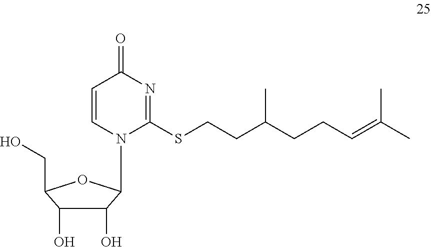 [SMILES: CC(C)=CCCC(C)CCSc1nc(=O)ccn1C1OC(CO)C(O)C1O]

To a stirred solution of nucleoside 21 (s²U, 5.0 mg, 0.019 mmol), 5-bromo-2-methylpent-2-ene (13.3 mg, 0.057 mmol) and DIPEA (7.0 mg, 0.057 mmol) in MeOH (0.5 mL). The mixture was stirred overnight at r.t. under N₂ atmosphere. The residue was purified by preparative thin layer chromatography and confirmed by ¹H NMR and MS (ESI).

¹H NMR (400 MHz, CDCl₃) δ 8.34 (d, J=8.2 Hz, 1H), 6.74 (d, J=8.4 Hz, 1H), 6.04-5.87 (m, 1H), 5.33 (s, 1H), 4.94 (dd, J=26.2, 14.0 Hz, 1H), 4.66 (d, J=12.8 Hz, 1H), 3.80-3.74 (m, 2H), 2.16 (s, 2H), 1.86 (s, 2H), 1.61 (s, 4H), 1.59 (s, 4H), 0.99 (d, J=6.6 Hz, 3H). MS (ESI): Calculated [M+H₂O+H]⁺=417.5. Found [M+H₂O+H]⁺=417.1.

SelU-mediated geranylation of tRNA in the fluorescent labeling investigations.

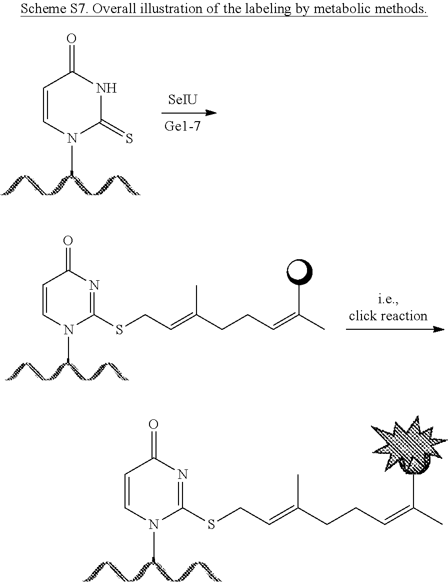

General procedure: Partially purified SelU-His₆ (15 μg, ca. 0.35 nmol), 20 μg (ca. 3.74 nmol) tRNA and geranyl pyrophosphate ammonium salt (5 eq. Sigma) dissolved in 100 μl of buffer containing 10 mM Tricine-KOH, pH=7.2, 0.2 mM dithiothreitol (DTT), and 100 mM MgCl₂, the sample mixture was then incubated at 25° C. for 24 h. The Ge-tRNA product was monitored by RP-HPLC using a Kinetex C₁₈ column (5μ, 100 Å) using the gradient parameters of buffer B: 0-10 min 0% B; 10-40 min 0-35% B; 40-45 min 35-100% B; 45-50 min, 100% B; 50-55 min, 100-0% B; 55-60 min, 0% B. Buffer A: 0.1 M CH₃CO₂NH₄; pH=6.8. Buffer B: 0.1 M CH₃CO₂NH₄ with 40% CH₃CN; the collected fraction was desalted and the geranylated product was tested by fluorescent labeling or MALDI-TOF MS.

Direct labeling of prenylated tRNA using this Ene-ligation.

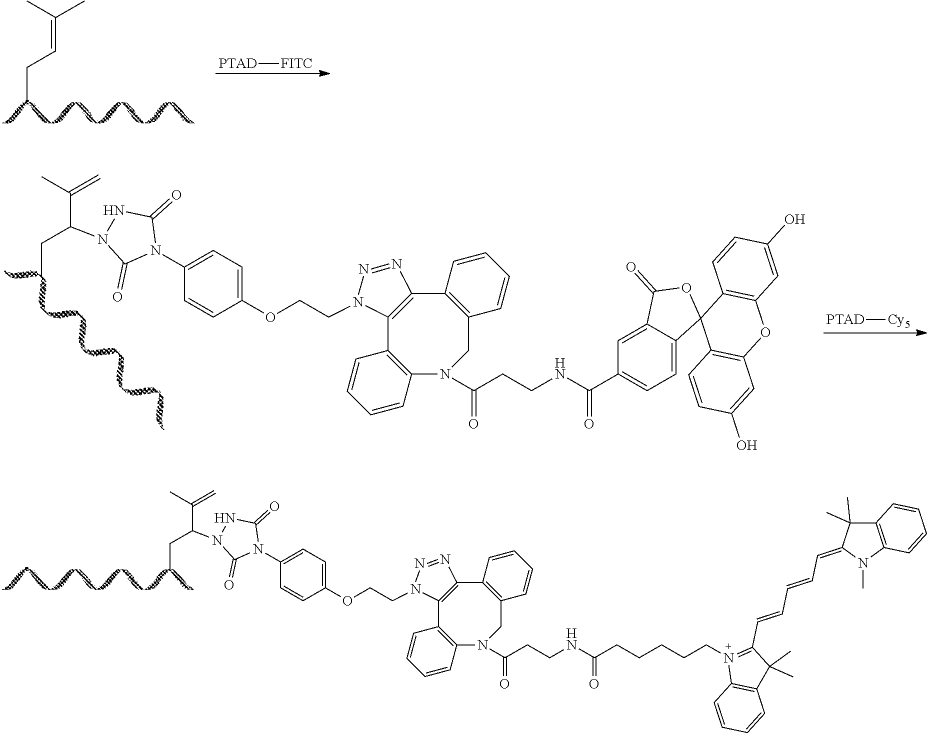

Scheme S8. Direct fluorescent labeling using fluorescent probes via Ene-ligation. Specifically, for prenylated substrates such as Ge1, Ge2 and Ge3.

### Analysis of Hydrolyzed Products by RNase T1.

RNase T1 is an endoribonuclease that specifically degrades single-stranded RNA at G residues. It cleaves the phosphodiester bond between 3′-guanylic residue and the 5′-OH residues of adjacent nucleotide with the formation of corresponding intermediate 2′, 3′-cyclic phosphate. The reaction products are 3′-GMP and oligonucleotides with a terminal 3′-GMP. RNase T1 does not require metal ions for activity.

tRNA^{Glu} from E. coli. mass analysis: Calculated MS 24441 (or 24124)+77.952+15.023+181.129 (or 225.119)−108.042−108.042+1004=24715.105/24579.233/24759.094/24442.094 (+1004). Found 25417.4251, 25724.55. It is reported that geranylated RNA in Escherichia coli, Enterobacter aerogenes, Pseudomonas aeruginosa and Salmonella enterica var. Typhimurium. Ana these geranylated nucleotides occur in the first anticodon position of tRNA^{Glu}_{UUC}, tRNA^{Lys}_{UUU}, tRNA^{Gln}_{UUG} at a frequency up to 6.7% (˜400 geranylated nucleotides per cell).

### Materials

Cas9 Nuclease, Streptococcus pyogenes (product #M0646), T7 Endonuclease I (product #M0302) Ribonucleotide solution mix (NTPs) and deoxy-ribonucleoside triphosphates (dNTPs) were purchased from New England Biolabs (USA). Transcript Aid T7 High Yield Transcription kit (product #K0441) and Glycogen (product #R0561) were purchased from Thermo Fisher Scientific. Pyrobest™ DNA Polymerase and PrimeSTAR HS DNA Polymerase were purchased from TaKaRa Shuzo Co. Ltd. (Tokyo, Japan). DNA Clean & Concentrator™-5 kit (product #D4014) was purchased from Zymo Research Corp. The DNeasy Blood & Tissue Kit was purchased from QIAGEN. The oligonucleotides at HPLC purity were obtained from TaKaRa Company (Dalian, China). The nucleic acid stains Super GelRed (No.: S-2001) was bought from US Everbright Inc. (Suzhou, China). DPBA (Cas #17261-28-8), TCEP (Cas #51805-45-9), 4-(2-hydroxyethyl)-piperazine-1-ethanesulfonic acid (HEPES, Cas #7365-45-9) and Thiazolyl Blue Tetrazolium Bromide (MTT, Cas #298-93-1) were purchased from Sigma-Aldrich Inc. (Shanghai, China). DPBS (Cas #63995-75-5) was purchased from TCI Development Co., Ltd (Shanghai). The concentration of DNA or RNA was quantified by NanoDrop 2000c (Thermo Scientific, USA). Gel Imaging was performed using Pharos FX Molecular imager (Bio-Rad, USA).

### Methods

### SelU Expression, and Purifications

Expression. Strain pET28b-SelU (BL21) was cultivated in Kan (kanamycin) LB overnight at 37° C., followed by inoculation into fresh 200 mL Kan LB at 220 r.p.m./min, 37° C. for about 3 h to OD=0.6. Subsequently, the protein expression was induced by adding 1 mmol/L IPTG at 220 r.p.m./min, 28° C. for 5 h.

Purifications. The His×6 Ni Gravity Column (1.0 ml) was equilibrated and all buffers were settled to the working temperature. The column was then washed with 5-10 column volumes of His×6 Ni Equilibration Buffer (50 mM sodium phosphate, 6 M guanidine-HCl, 300 mM NaCl, 20 mM imidazole; pH=7.4). The clarified sample was added to the column and sealed with column stopper. The target protein was allowed to bind by slowly inverting the column for 1 h. The bounded protein in the resin was then settled to the bottom of the column by placed the column in a standing position. The column was washed with 10 column volumes of His×6 Ni Equilibration Buffer followed by 10 column volumes of His×6 Ni Wash Buffer (50 mM sodium phosphate, 6.0 M guanidine-HCl, 300 mM NaCl, 40 mM imidazole; pH=7.4). The target protein was eluted with approximately 10 column volumes of Elution Buffer (50 mM sodium phosphate, 6 M guanidine-HCl, 300 mM NaCl, 300 mM imidazole; pH=7.4) and collect 1 mL per tube. The sample was analyzed and identified by 5% agarose gel electrophoresis.

### NMR Spectroscopy

Measurements were carried out using 1H, 13C NMR, and 31P NMR. Chemical shifts (δ) were reported in parts per million (ppm) and referenced to the solvent signals. Data are reported as follows: chemical shift, multiplicity (s=singlet, d=doublet, t=triplet, and m=multiplet) and coupling constants (in hertz). Bruker AM-400 spectrometer (400 MHz) and a Bruker AVANCE NEO 600 (600 MHz) was used for NMR data collection and spectral interpretation.

### General Synthetic Procedures for Pyrophosphates (Ge1-7, Scheme S1-5)

Typical procedure: To a stirred solution of (E)-1-bromo-3,7-dimethylocta-2,6-diene (50.0 mg, 0.220 mmol) in CH₃CN (1.5 ml) was slowly added tris (tetra-N-butylammonium) hydrogen pyrophosphate (200 mg, 0.264 mmol). The mixture was stirred for 5 h at room temperature under nitrogen atmosphere. The crude product was purified by HPLC and lyophilized to dryness.

Molecular Docking Study of SelU with Pyrophosphates Ge

The molecular docking studies were performed by using AutoDock Vina 1.1.2. Ref. M1 The ligand sites were detected by using AutoLigand, Ref. M2 and the ligands (pyrophosphate Ge1/Ge5/Ge6) site containing Cys97 and Gly67 was used to define the binding pocket by establishing a grid box centered on X: −26.5 Y: 2.6 Z: 7.8 Å with the dimensions of X: 26.9 Y: 26.0 Z: 19.1 Å. The magnesium ion was first docked into the binding pocket. The docked position of the magnesium ion with the lowest docked energy was selected to prepare a protein-ion complex. Then each ligand was docked into the binding pocket with the presence of the magnesium ion. The docking poses of ligands were also selected for subsequent analyses according to the lowest docked energies. Top references:
M1. Trott, O.; Olson, A. J., AutoDock Vina: improving the speed and accuracy of docking with a new scoring function, efficient optimization, and multithreading. J. Comput. Chem. 2010, 31, 455-61.
M2. Harris, R.; Olson, A. J.; Goodsell, D. S., Automated prediction of ligand-binding sites in proteins. Proteins 2008, 70, 1506-17.

General experimental procedures for SelU-mediated geranylation Enzymatic fluorescent labelling analysis with pyrophosphates Ge1-7.

Partially purified SelU-His×6 (15 μg, ca. 0.35 nmol), 20 μg (ca. 3.74 nmol) tRNA and geranyl pyrophosphate ammonium salt (Sigma, 5 equiv.) dissolved in 100 μl of buffer containing 10 mM Tricine-KOH, pH=7.2, 0.2 mM dithiothreitol (DTT), and 100 mM MgCl2, and the sample was incubated at 25° C. for 24 h. The Ge-tRNA product was monitored by RP-HPLC using a Kinetex C18 column (5μ, 100 A, 150×4.6 mm) using the gradient parameters of Buffer B: 0-10 min 0% B; 10-40 min 0-35% B; 40-45 min 35-100% B; 45-50 min, 100% B; 50-55 min, 100-0% B; 55-60 min, 0% B. Buffer A: 0.1 M CH3CO2NH4; pH=6.8. Buffer B: 0.1 M CH3CO2NH4 with 40% CH₃CN; the collected fraction was desalted and the geranylated product was tested by fluorescent labelling or MALDI-TOF MS.

With pyrophosphates Ge2, 3, 7.

RNase T1 is an endoribonuclease that specifically degrades single-stranded RNA at G residues. It cleaves the phosphodiester bond between 3′-guanylic residue and the 5′-OH residues of adjacent nucleotide with the formation of corresponding intermediate 2′, 3′-cyclic phosphate. The reaction products are 3′-GMP and oligonucleotides with a terminal 3′-GMP. RNase T1 does not require metal ions for activity. The Ge-tRNA product was hydroxylated to give nucleosides with varied modifications, which were further analyzed by AB Sciex 4500, Wuhan biological sample 4500 Q-trap (Wuhan Biobank Co., Ltd).

With pyrophosphates Ge1, 5, 6.

SelU-catalyzed tRNA geranylation with pyrophosphates Ge5-7 as substrates, the in-gel fluorescent imaging was performed therefore. In the fluorescent labelling studies when using Ge5-6 and Ge7 as substrates, DBCO-Cy5 was used for labelling of Ge5 or Ge6, while BODIPY-R6G-N3 was used for labelling of Ge7. Confocal parameters are: Blue channel (E.X. 492 nm. E.M. 507 nm), red channel (E.X. 649 nm. E.M. 660 nm) and green channel (nm). See Scheme S7 for details.

Direct fluorescent labelling of prenylated tRNA by using PTAD-DBCO-Cy5 probe.

tRNA (1.0 mg) isolated from E. coli using Zymo purification kit strictly following the instruction protocol was dissolved in PBS buffer (0.1 M, pH=7.4) and the PTAD-DBCO-Cy5 probe (1.0 mM) generated by in-situ oxidation with N-bromosuccinimide (NBS, 1 M in DMF) was added in one portion at 4° C. The mixture was incubated at 4° C. for 15 min in a sterile tube. And excess fluorescent probe was removed by 3K filter (Millipore) and the resulting nucleic acid was analyzed by in-gel fluorescence (Scheme S8).

Mass Spectra identification of prenyl-containing nucleic acid labelled with PTAD-DBCO-Cy5.

tRNAGlu from aforementioned procedure was further analyzed by MALDI-TOF/TOF mass spectra (AXIMA-PerformanceMA, Qinghua University). The details are presented below:

### MALDI Matrix Application

3-Hydroxypicolinic Acid (3-HPA, Sigma Aldrich, USA) was chosen as MALDI matrix for DNA detection. The matrix solution was prepared by dissolving 20 mg 3-HPA and 45 mg dihydrogen ammonium citrate (DHAC) in 1 mL mixture solution of 50% acetonitrile/50% water. A home-built inkjet printing device was developed to print an array of matrix droplets on sample. This device consisted of a piezoelectric inkjet print head for matrix solution ejection (Fuji Electrics Systems Co., Ltd, Japan) and an XY-motorized stage where the ITO glass was placed (MMU-30X, Chuo Precision Industrial Co., Ltd, Japan). A laboratory-made software was used to control the inkjet waveform and the movement of x-y stage. ITO glass with cells was firstly washed with 100 mM ammonium acetate solution to remove non-volatile salts which might affect ionization efficiency. After air drying, a 6×6 matrix array of circular regions ˜300 μm in diameter was printed onto the ITO glass and imaged with fluorescent microscope (DMI 4000B, Leica, Germany). For quantitative analysis, DNA internal standard and matrix solution were loaded into different channels of the inkjet printing head. The internal standard was printed prior to the matrix onto the same position.

### MALDI-MS Analysis

MALDI-MS analysis was performed in an AXIMA Performance MALDI-TOF/TOF mass spectrometer (Shimadzu Co. Ltd., Japan). This instrument was equipped with a 337 nm nitrogen laser. ITO glass with cells was attached to the stainless MALDI plate by conductive tapes. Data were acquired in a linear negative mode and signals between m/z 20000-30000 were collected. Raster scans on cell surfaces were performed automatically using the mass spectrometry software (Shimadzu Biotech., Japan). The scan area was 200 μm×200 μm with the sampling distance of 50 μm. For each coordinate, mass spectra resulting from 20 laser shots at 5 Hz were accumulated to obtain an average mass spectrum.

### HEK293T Cell Culture and Plating

The HEK293T cells were washed twice with PBS, and trypsin was added for 1 min. Then 2.0 mL DMEM medium was added to the mixed well, followed by centrifuged at 1,000 r.p.m for 5 min, and the supernatant was discarded. Fresh medium was added to resuspend the cell pellet and cells were seeded in a confocal dish at the density of 0.5×1 05, the cells were gently mixed using pipette, and then placed in CO2 incubator.

### Transfection of HEK293T Cells

150 mM sterile NaCl solution was prepared as a diluent for DNA and Vigofect (Vigorous Biotechnology Beijing Co., Ltd.). The culture medium in the confocal dish was then replaced with fresh complete culture medium before transfection, and the culture was incubated at 37° C. under 5% CO2 atmosphere. The pCMV-Myc-SelU-GFP group, pCMV-Myc-SelU-5s-GFP and Ge1 or Ge5 or Ge6 group were set as control groups.

Configure the transfection working solution. First, 2.5 μg pCMV-Myc-SelU-GFP or pCMV-Myc-SelU-5s-GFP was added into the diluent (total 100 μl) and stored at room temperature for 5 min. Next, 2.5 μl VigoFect was added to the diluent (total 100 μl), and the solution was mixed gently and placed at room temperature for 5 min. Next, the diluted VigoFect was added into the diluted DNA solution gently, and the resulting transfection working solution was incubated at room temperature for 15 min. Subsequently, the working solution was added to the cell culture, mixed gently and incubated for 24 h. 24 hours post transfection, the pyrophosphates Ge1, Ge5, Ge6 (1 mM) were added into the medium for 4 h. Cells were washed with PBS 3 times and then fixed with 4% paraformaldehyde for 15 min, and subsequently permeabilized with 0.5% Triton X-100 for 15 min. Next, TMSN3 (50 μM) and Selectfluor (50 μM) were added into Ge1 group for 30 min, cells were then incubated with DBCO-Cy5 (5 μM) for 30 min and then stained with 4′, 6-diamidino-2-phenylindole (DAPI) for 15 min in darkness at room temperature (RT). Samples were washed with PBS three times and examined under a confocal microscope (LSM780, Carl Zeiss).

Amplification, Expression, and Extraction of pCMV-Myc-SelU-GFP, pCMV-Myc-SelU-5s-GFP Plasmid

pCMV-Myc-SelU-GFP or pCMV-Myc-SelU-5s-GFP plasmid was transformed into competent DH5α cells at 4° C. Followed by incubation in an ice-water bath for 30 min, heat shocked at 42° C. for 90s, then placed on ice for another 5 min. 400 μl of LB solution was added and the cells were incubated at 37° C., 220 r.p.m. for 45 min. Finally, the bacterial solution was spread onto the Kan-containing LB solid culture plate and incubated at 37° C. for 12-16 h until a single colony appeared. Next, in sterile surface, a single colony was picked and inoculated into 5 mL of LB solution (kan), incubated overnight at 37° C., 220 r.p.m., until the culture solution appeared turbid, followed by plasmid extraction.

### Synthesis of the Standard Nucleosides.

General Procedure: In an anhydrous DMF (0.1 ml) solution of s2U (13.0 mg, 0.05 mmol), K2CO3 (0.2 mmol) was introduced at room temperature. A single portion of bromide (0.2 mmol) was then added, and the mixture was heated to 85° C. for overnight stirring. Solvents were subsequently evaporated under vacuum, and the residue was further purified by HPLC to yield the Ge-s2U product.

### Photo Linking Substrate (Ge4) for Reader Enzyme Evaluation

Co-incubation experiment of pET28b-SelU lysate and Ge4 (benzophenone pyrophosphate).

### General Procedure:

(1) Add the E. coli bacterial cells to the lysis solution (Western and IP lysis buffer, Beyotime), sonicate for 5 min (80W, work 10 seconds, stop 10 seconds), lyse until the sterile body precipitates, centrifuge at 10,000×g for 15 min, collect the supernatant, and determine the protein concentration by BCA kit.

(2) Add appropriate amount of Ge4 (benzophenone pyrophosphate) to the protein lysate and incubate overnight at 4° C. with UV (365 nm) irradiation on ice for 15 min.

(3) Take 50 μl of probe from each tube of sample and add excess NBS to oxidize (the reaction solution turns pink when upon addition), incubate the PTAD-DBCO-biotin probe with the above-mentioned lysis solution for 4 h (4° C.). Add 50 μl beads to the EP tube and place the tube on the magnetic stand, discard the supernatant, add buffer 2 to wash the beads twice, then add the lysate to the washed beads. Incubate overnight with rotation at 4° C., when complete incubation, place the tube on a magnetic stand, wash the complex with eluent 3 times, add 20 μl protein loading buffer, centrifuge at 14,000×g for 30s, denaturized at 95° C. for 5 min, centrifuge at 14000×g 1 min, then analyze the supernatant by SDS-PAGE electrophoresis (10%).

After mixing the above reagents thoroughly, cast the gel, add isopropanol to even the gel, solidify at r. t. for 30 min, then discard the isopropanol, and blot the remaining reagents with filter paper.

Stain the gel with Coomassie Brilliant Blue staining solution on a shaker for 30 min and destain with the decolorizing solution until the protein bands appear. The result is shown in the Fig. S9.

Cell-specific labeling of SelU-mediated tRNA geranylation.

### General Protocol:

### HEK293T Cell Culture.

1. HEK293T cell culture and plating. wash the HEK293T cells twice with PBS, add trypsin to break the adhere cells from vessel for 1 min. neutralize with 2.0 mL of DMEM medium, mix well then centrifuged at 1,000 r.p.m for 5 min and discard the supernatant. In sterilize table, add fresh medium to resuspend the cell pellet and count, seed the cells in confocal dish at the density of 0.5×105, mix the cells gently by pipetting, then place them in CO2 incubator.
2. Transfection of HEK293T cells. Prepare 150 mM sterile NaCl solution as a diluent for DNA and Vigofect., exchange the culture with fresh medium in the confocal dish before transfection, and incubate at 37° C., 5% CO2. The plasmids for transfection are pCMV-Myc-SelU-GFP group, pCMV-Myc-SelU-5s-GFP and Ge1 or Ge5 or Ge6 group. Prepare the transfection working solution:
3. Take 2.5 μg pCMV-Myc-SelU-GFP or pCMV-Myc-SelU-5s-GFP, add the diluent to 100 μl and store at room temperature 5 min.
4. Add 2.5 μl VigoFect to 100 μl diluent, mix gently and place at room temperature for 5 min.
5. Add the diluted VigoFect to the DNA solution, and place the resulting solution at room temperature for 15 min.
6. Add working solution to the culture solution, mix gently and incubate for 24 h. 24 h post transfection, add Ge1 [symbol] Ge5 [symbol] Ge6 (1 mM) to the medium incubate for 4 h. Wash the cells with PBS 3 times, fix with 4% paraformaldehyde for 15 min, and subsequently permeabilize with 0.5% Triton X-100 for 15 min. Add the TMSN3 and Selectfluor (50 μM) to Ge1 group for 30 min. Incubate the cells with DBCO-Cy5 for 30 min before stained with 4′, 6-diamidino-2-phenylindole (DAPI) for 15 min in darkness at r.t. Wash the samples with PBS 3 times and examine under a confocal microscope (LSM780, Carl Zeiss).

Amplification, expression, and extraction of pCMV-Myc-SelU-GFP, pCMV-Myc-SelU-5s-GFP plasmid.
1. pCMV-Myc-SelU-GFP or pCMV-Myc-SelU-5s-GFP transformation. Thaw the competent DH5α cells on ice. Add 1 μl plasmid to the DH5α cells, mix gently and bath in ice for 30 min. Heat shock at 42° C. for 90s, and quickly place on ice for 5 min. Add 400 μl of LB solution, shake at 37° C., 220 r.pm./min for 45 min, spread the bacterial solution onto the Kana-containing LB solid culture plate. Place it in 37° C. incubator, 12-16 h later until a single colony appears.
2. After 16 h, inoculate a single clone into 5 mL of LB solution (Kana), incubate overnight at 37° C., 220 r.p.m./min, until the culture solution becomes turbid followed by plasmid extraction.
3. Plasmid extraction is carried out by using Dynake Biological Plasmid Extraction Kit

(1) Add 250 μl of Buffer BL to the adsorption column AC and centrifuge at 12,000×g for 1 min to activate the silica gel membrane.

(2) Take 4 ml of the overnight bacterial cultured, centrifuge at 12,000×g for 1 min, and collect the bacterial cells.

(3) Add 200 μl Buffer S1 to resuspend the bacterial pellet, vortex and shake until a sterile block is reached.

(4) Add 200 μl Buffer S2, mix well by invert the column 7 times to fully lyse the bacteria.

(5) Add 200 μl Buffer S3, mix well by invert the column 7 times. The solution should turns into white flocculent precipitate. Centrifuge at 12,000×g for 15 min.

(6) Aspirate the supernatant carefully, transfer the supernatant to the adsorption column AC, centrifuge at 12000×g for 1 min, discard the waste liquid, and put the adsorption column AC back into the empty collection tube

(7) Add 700 μl Buffer W2 to the adsorption column AC, centrifuge at 12,000×g for 1 min, and discard the waste liquid. Repeat this step.

(8) Put the adsorption column AC back into the empty collection tube and centrifuge at 120,00×g for 2 min.

(9) Place the adsorption column AC in a clean 1.5 mL centrifuge tube, let it stand at 25° C. for 2 min, add 30 μl of Eluent to the middle of the adsorption membrane, let it stand at 25° C. for 2 min, then centrifuge at 12,000×g for 2 min. Obtain the plasmid, and determine the concentration.

### Results

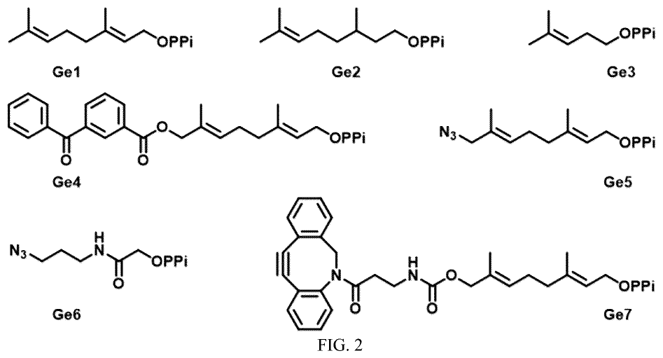

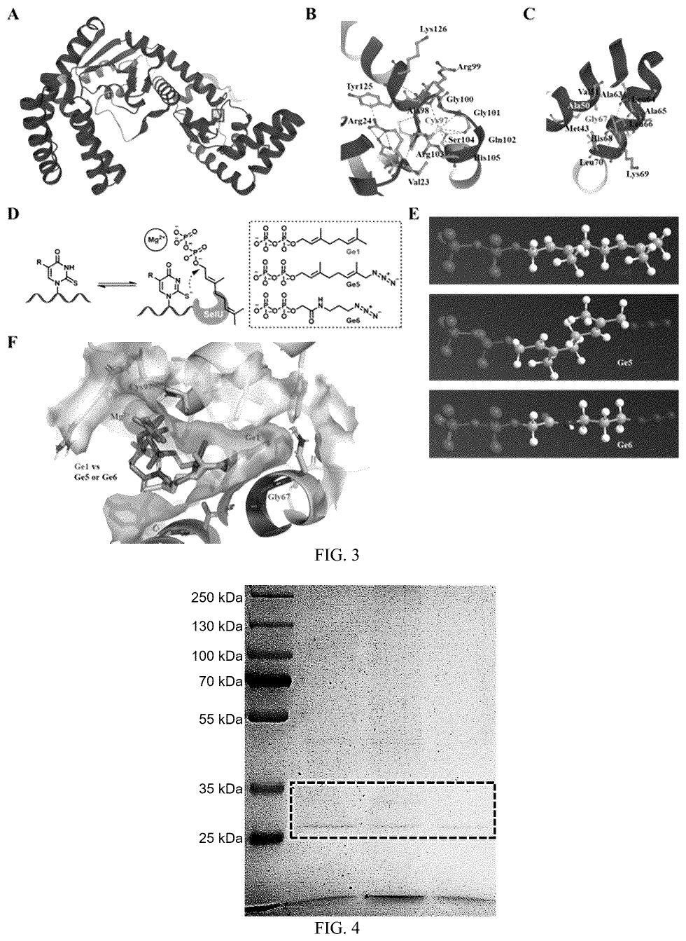

To test the viability of geranyl tRNA tagging, we first synthesized a series of analogues including geranyl pyrophosphate variants Ge2 and Ge3, a diphenylketone analogue Ge4, azido analogues Ge5 and Ge6, and a DBCO derivative Ge7 (FIG. 2 and Schemes S1-5). The pyrophosphate Ge4 was designed as a photo-crosslinker, and all other pyrophosphates were designed to mimic the natural geranyl pyrophosphate substrate Ge1. Synthesis of Ge1, Ge2, and Ge3 were all made from the corresponding bromide, and the crude products were purified by preparative HPLC. Synthesis of Ge6 followed a two-step protocol. First, 3-azidopropylamine was reacted with 2-bromoacetyl bromide to yield bromide intermediate with 90% of yield, which was further reacted with hydrogen pyrophosphate to give Ge6. The analogs Ge4, Ge5, and Ge7 were synthesized from geranyl acetate. Selenium dioxide and tert-butyl peroxide were used to oxidize the terminal methyl group on allylic position of geranyl acetate to generate the hydroxy intermediate. For Ge4, the diphenylketone acid was reacted with the hydroxide to give the ester. Deprotection of the acetyl group, bromination using tribromophosphine, and a treatment with hydrogen pyrophosphate in a three-step domino process with final HPLC purification yielded pure Ge4 (Scheme S2). For the synthesis of Ge5, a key intermediate hydroxide was subjected to the treatment with mesyl chloride and subsequently with sodium azide to generate the azido-intermediate. The aforementioned three-step domino process were subsequently applied to produce final Ge5 (Scheme S3). Similarly, a key intermediate hydroxide was subjected to react with triphosgene and DBCO-amine, followed by the three-step domino process to generate Ge7 (Scheme S5). All the compounds were analyzed by mass spectroscopy, ¹H NMR, and ³¹P NMR, confirming the identity of the desired products. Like the natural Ge1 [−6.85 (d, J=22.68 Hz), −10.57 (d, J=22.68 Hz)], the analogs Ge5 and Ge6 have characteristic peaks in the NMR spectra at −6.64 (d, J=22.68 Hz), −10.44 (d, J=22.68 Hz), −5.87 (d, J=22.7 Hz), and −11.19 (d, J=22.3 Hz), which are typical for pyrophosphates (FIG. 3D).

Following that, we expressed and purified SelU, the enzyme that installs the geranyl modification on tRNAs. Immunoblotting analysis confirmed the successful expression of SelU-His6 with the His-tag used for the affinity column purification (FIG. 4). To investigate the substrate recognition criteria and to better understand the geranylation process, we used AlphFold (DeepMind) to predict the 3D structure of SelU. The protein is predicted to adopt an overall α helical shape (FIG. 3A). SelU has a rhodanese domain, the key working domain for both geranylation and selenation processes,³ as well as a P-loop domain with a Walker A motif found in many ATP- and GTP-binding proteins and is also involved in substrate binding. Cys97 and Gly67, the two crucial residues in the rhodanese domain, have been shown to play roles in both substrate binding and geranylation activation. As a result, the precise interaction network of Cys97 and Gly67 with neighboring residues was tested (FIGS. 3B-3C), which is compatible with the reported experimental data.³ We conducted molecular docking investigations with the synthesized geranyl-pyrophosphate ligand analogues using this apo-model. The results showed that the predicted ligand binding site of SelU can well accommodate Ge1, Ge5, and Ge6, and that the azido analogues are ideal substrates for geranylation by SelU (FIGS. 3E-3F). Although more precise SelU-ligand interactions with high-resolution complex structures remain elusive, this AI-based prediction and docking technique provides some general directions for the mechanistic studies and additional ligand design based on structurally unknown proteins.

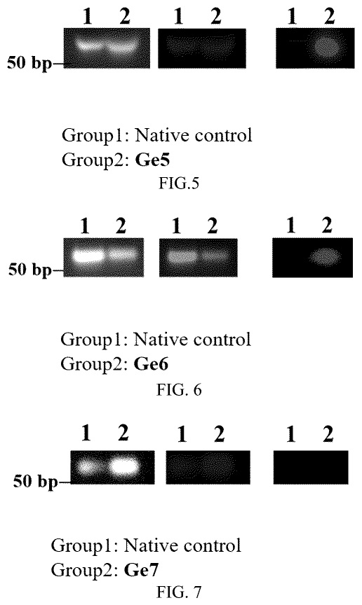

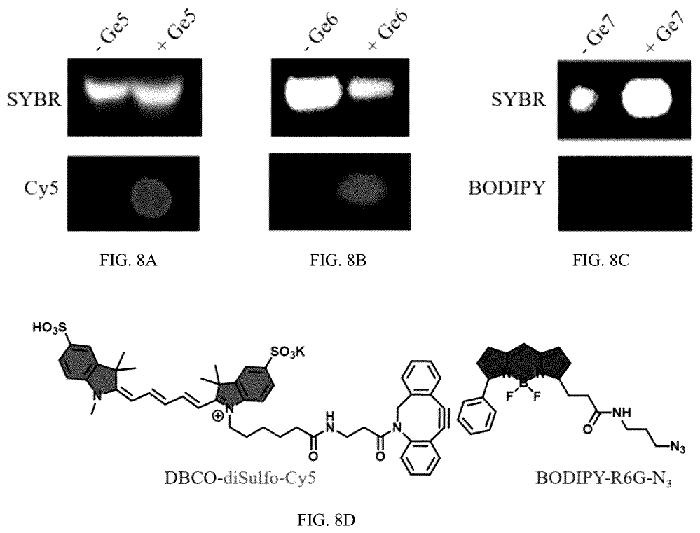

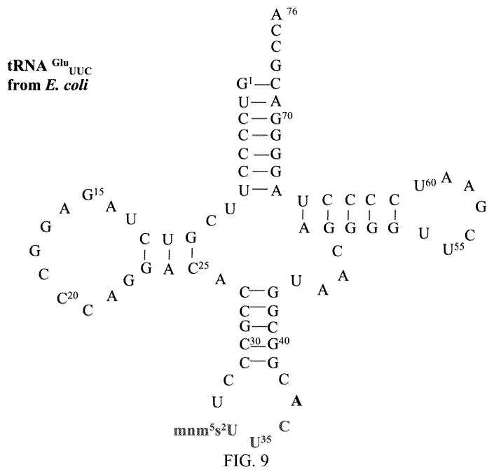

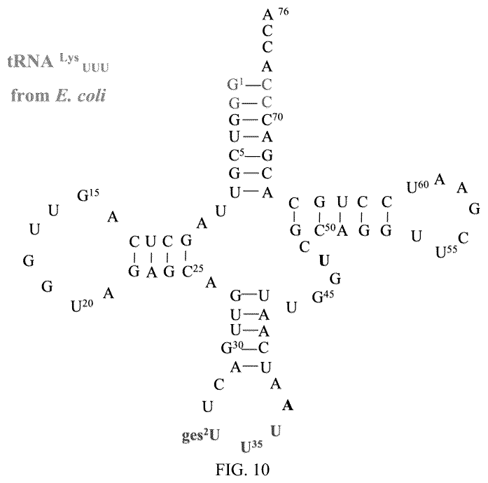

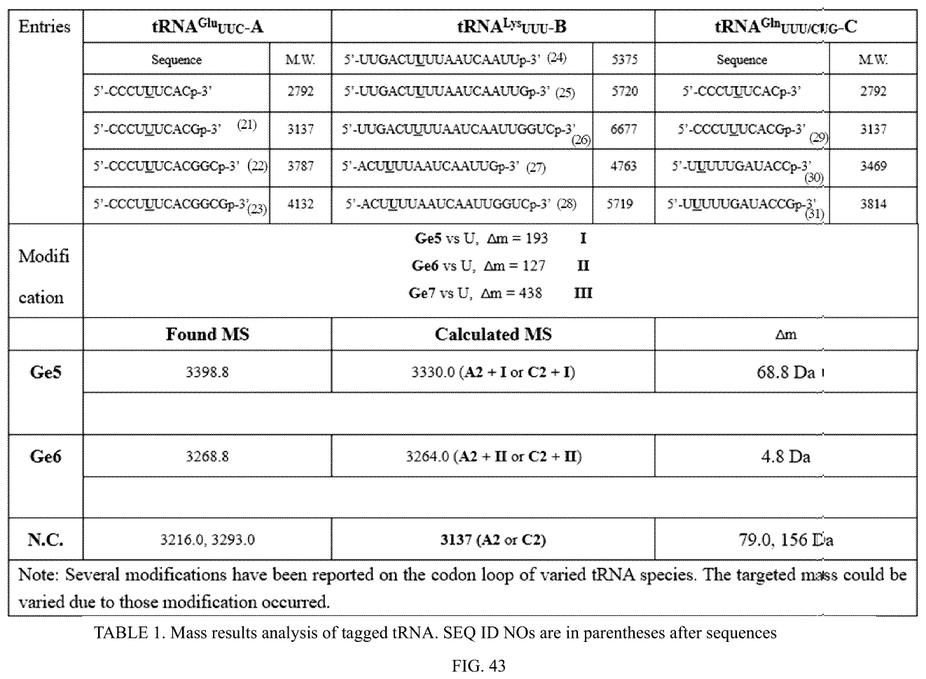

With these ‘clickable’ tags being recognized by SelU, we further conducted the fluorescent labelling of tRNA^{Gln, Glu, Lys} in the presence of the ligands Ge5, Ge6, Ge7 and the purified SelU (the detailed processes are shown in Scheme S7, FIGS. 5-7, and Table 1 in FIG. 43). When the reaction was performed in the presence of azido-pyrophosphate Ge5, efficient fluorescent signal of tRNA was observed with DBCO-Cy5 treatment, as shown in FIGS. 5 and 8. SYBR green staining was used to validate the presence of RNAs. Similarly, Ge6 is also a substrate for SelU, and the subsequent click reaction with DBCO-Cy5 resulted in fluorescent tRNAs (FIGS. 6 and 8B). After the labelling, the tRNAs were further treated with RNase T1 and analyzed by LC-MS/MS to confirm the presence of the fluorescent groups on nucleosides (Table 1 in FIG. 43 and Scheme S6 for detailed standard nucleoside synthesis). The mass fragmentation results (FIGS. 9-11), including 3398.8 (CCCUUUCACG (SEQ ID NO: 4), Ge5), 3268.8 (ACUUUUAAUCAAUUG (SEQ ID NO: 5), Ge6) and 3216.0/3293.0 (UUUUUGAUACCG (SEQ ID NO: 6), Ge1), were assigned as the corresponding RNA fragments cleaved at the G sites, which are the target cleavage sites of the RNase T1. All these data support the idea that our ligands Ge5 and Ge6 can be successfully transferred to tRNA as tags for further fluorescence labelling. On the other hand, Ge7 bears a large DBCO moiety and most likely interferes with the SelU binding, as a result the fluorescent click labelling using BODIPY-R6G-N₃ failed (FIGS. 7 and 8C). Similarly, we also evaluated the labelling feasibility of ligands Ge2 and Ge3. After the click reaction and RNase cleavage, both HPLC and MS-spec data showed no modified RNA residues, indicating that neither of these two substrates can be recognized by SelU, probably due to the higher flexibility of Ge2 and shorter chain length of Ge3.

This two-step indirect ACT-Flu labelling has great application potential in targeting lipidated RNAs in both healthy and disease eukaryotic cells. Recently, it was shown that the levels of thiolated wobble uridines change in response to hydroxyurea in budding yeasts.⁴ Similarly, it was discovered that paromomycin therapy altered the amounts of thiolated-tRNA in human colorectal cancer cells. In addition, the human U34 writers related to mcm⁵s²U have also been shown to confer resistance to targeted therapy in melanomas.⁴³ As a result, our labelling approach could be used to research and track the quantities of these thiolated tRNAs under various cellular conditions and environmental stresses. In summary, these experiments allowed us to identify two pyrophosphate analogues, Ge5 and Ge6, which are suitable for SelU-mediated tRNA geranylation and fluorescent labelling. This two-step process, azidation and click tagging of fluorescent dye (ACT-Flu), may be very effective for in-vitro tRNA labelling and detecting.

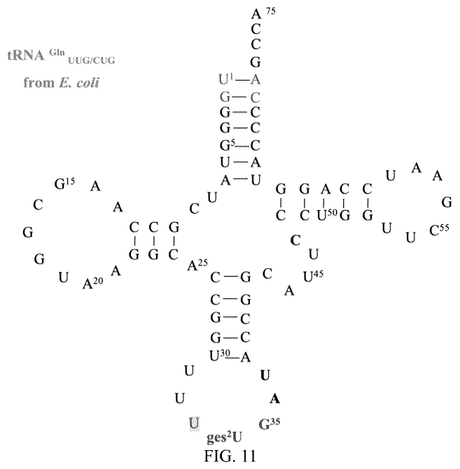

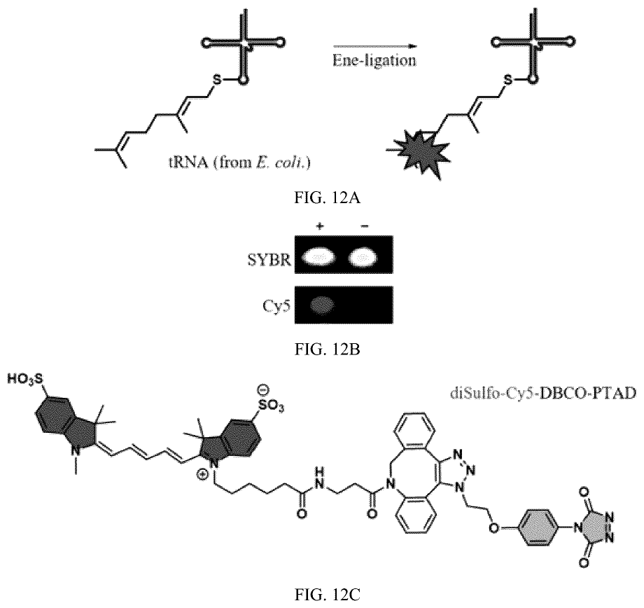

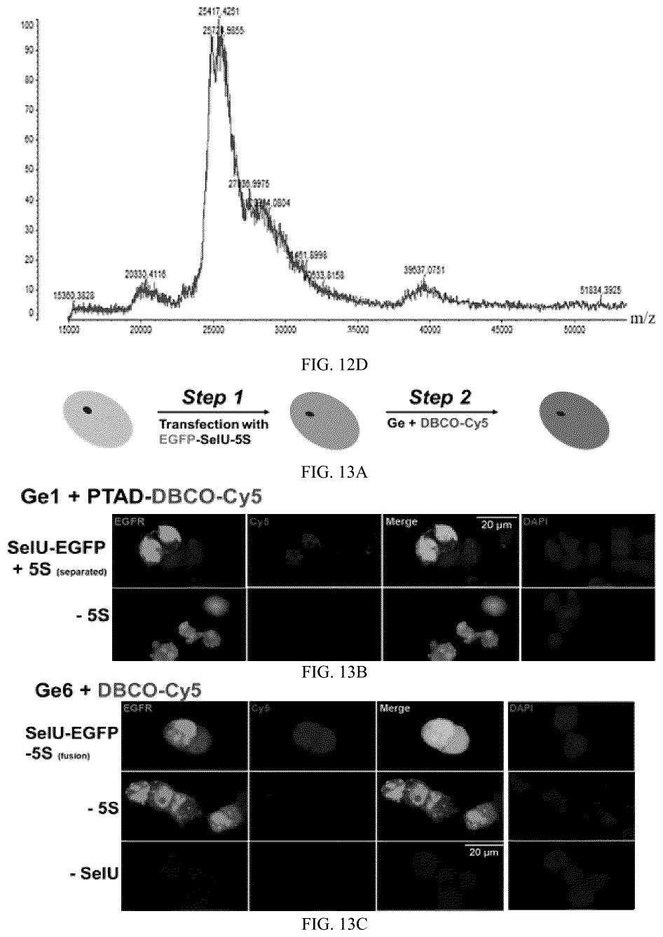

We also accessed the feasibility of a direct labelling of prenyl-containing RNAs. Since some bacterial tRNAs contains geranyl groups on the wobble position of ³⁴U, we investigated if a 4-phenyl-1,2,4-triazoline-3,5-dione (PTAD) activated fluorescence dye molecule could be directly attached to these terpene terminals through a known Ene-reaction, where PTAD reacts with conjugated diene group³⁵⁻³⁷ (FIG. 12A). Indeed, as shown in FIG. 12B, a red fluorescent signal was observed when the total RNAs isolated from E. coli were treated with 10 μM of PTAD-DBCO-Cy5 (structure shown in FIG. 12C, also see FIG. 11), whereas there was no red fluorescence in the absence of this fluorophore. SYBR staining confirmed that the same amount of tRNAs were loaded in gel. We next applied this fluorophore probe directly to the ges²U-tRNA to test the labelling specificity. The labelled tRNAs were analyzed by MALDI-TOF (FIG. 12D). Mass peaks detected at 25724.9355 and 25417.4251 amu corresponded to PTAD-DBCO-Cy5-labeled tRNA^{Gln, Glu, Lys}. Briefly, the direct labelling of geranylated tRNA by the fluorescent probe PTAD-DBCO-Cy5 could be achieved based on the in vitro Ene-ligation.

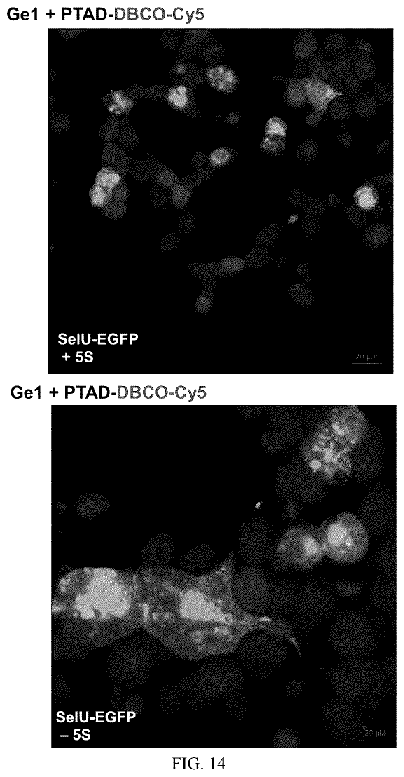

Next, we constructed the SelU-pCMV-Myc-EGFP (5593 bp), which contains E. coli SelU tagged with EGFP, and the 5s-pCMV3 (6333 bp, which contains 5S rRNA) plasmids in order to further apply our methodologies to in-cell tRNA labelling. We then tested the in vivo fluorescent labeling of geranylated tRNAs with pyrophosphates Ge1 coupled with the direct labeling (FIG. 13A). In particular, the prenylated tRNAs catalyzed by SelU in the presence of unprocessed geranyl pyrophosphate Ge1 in the HEK293T cell line were selectively tagged using the direct probe PTAD-DBCO-Cy5. The prenylated RNAs were directly labelled in this manner with high efficacy. The confocal image clearly showed the red fluorescence when transfected SelU, 5s (tRNA^{Lys}) with pyrophosphate Ge1, as well as probe PTAD-DBCO-Cy5 (FIGS. 13B and 14). Alternatively, in order to test the sequential two-step ACT-Flu approach, we also co-transfected Sell/with pyrophosphate Ge5 or Ge6, as well as the probe DBCO-Cy5 into HEK293T cell line. The resulting confocal image also displayed strong red florescence to indicate the successful labeling of the prenyl group (FIG. 15). When the SelU-pCMV-Myc-EGFP-5s plasmid was transfected in the presence of Ge6, both red and yellow fluorescence could be observed (FIG. 13C). As the control, no red fluorescence signal was detected in the absence of either SelU or 5s. These combined results demonstrated the successful in-cell labeling capacity of our two methodologies.

### Conclusions

In conclusion, we present two chemical labelling strategies for lipid-modified RNAs by taking advantage of the natural SelU-mediated tRNA geranylation process and the unique chemical reactivity of prenyl-groups. We synthesized a series of ‘clickable’ geranyl pyrophosphate analogues and discovered two candidates Ge5 and Ge6 feasible for indirect in-cell RNA labelling via a two-step process, azidation-and-click tagging of fluorescent dye, namely the ACT-Flu technique. Fluorescent microscopy and molecular simulation studies both confirmed that the two ligands could serve as ideal SelU substrates in the tRNA geranylation process, thereby providing a new toolset for detecting and monitoring the 2-thiouridine residue, which is an important biomarker of bacterial infections and cancers. In addition, we developed a direct metabolic incorporation and biorthogonal tagging (MIBT-Tag) method using the PTAD-based Ene-ligation chemistry of the prenyl group. Both approaches have been applied successfully for in-cell RNA fluorescent labelling. The biological importance of prenylation process has been increasingly appreciated with recent studies of OAS1, the double stranded RNA sensor that activates RNase L, indicating that the OAS1 mediated prenylation^{44, 45} process protects patients from severe illness infected by SARS-CoV-2 virus. The biochemical toolsets developed in our work have significance in vivo application potential. They will not only provide insights into the roles of prenylation modifications in gene regulation and RNA biology, but will also have applications as biomarkers specifically for prenylation levels in both healthy and diseased cells.

### REFERENCES

1. Adams, J. M.; Cory, S., Modified nucleosides and bizarre 5′-termini in mouse myeloma mRNA. Nature 1975, 255 (5503), 28-33.
2. Strobel, E. J.; Watters, K. E.; Loughrey, D.; Lucks, J. B., RNA systems biology: uniting functional discoveries and structural tools to understand global roles of RNAs. Curr Opin Biotechnol 2016, 39, 182-191.
3. Roundtree, I. A.; Evans, M. E.; Pan, T.; He, C., Dynamic RNA Modifications in Gene Expression Regulation. Cell 2017, 169 (7), 1187-1200.
4. Liu, N.; Dai, Q.; Zheng, G.; He, C.; Parisien, M.; Pan, T., N (6)-methyladenosine-dependent RNA structural switches regulate RNA-protein interactions. Nature 2015, 518 (7540), 560-4.
5. Barbieri, I.; Kouzarides, T., Role of RNA modifications in cancer. Nat Rev Cancer 2020, 20 (6), 303-322.
6. Li, J.; Zhu, W. Y.; Yang, W. Q.; Li, C. T.; Liu, R. J., The occurrence order and cross-talk of different tRNA modifications. Sci China Life Sci 2021, 64 (9), 1423-1436.
7. Jia, G.; Fu, Y.; Zhao, X.; Dai, Q.; Zheng, G.; Yang, Y.; Yi, C.; Lindahl, T.; Pan, T.; Yang, Y. G.; He, C., N⁶-methyladenosine in nuclear RNA is a major substrate of the obesity-associated FTO. Nat Chem Biol 2011, 7 (12), 885-7.
8 Zhao, B. S.; Roundtree, I. A.; He, C., Post-transcriptional gene regulation by mRNA modifications. Nat Rev Mol Cell Biol 2017, 18 (1), 31-42.
9. Frye, M.; Harada, B. T.; Behm, M.; He, C., RNA modifications modulate gene expression during development. Science 2018, 361 (6409), 1346-1349.
10. Yang, X.; Yang, Y.; Sun, B. F.; Chen, Y. S.; Xu, J. W.; Lai, W. Y.; Li, A.; Wang, X.; Bhattarai, D. P.; Xiao, W.; Sun, H. Y.; Zhu, Q.; Ma, H. L.; Adhikari, S.; Sun, M.; Hao, Y. J.; Zhang, B.; Huang, C. M.; Huang, N.; Jiang, G. B.; Zhao, Y. L.; Wang, H. L.; Sun, Y. P.; Yang, Y. G., 5-methylcytosine promotes mRNA export—NSUN2 as the methyltransferase and ALYREF as an m(5)C reader. Cell Res 2017, 27 (5), 606-625.
11. Chen, X.; Li, A.; Sun, B. F.; Yang, Y.; Han, Y. N.; Yuan, X.; Chen, R. X.; Wei, W. S.; Liu, Y.; Gao, C. C.; Chen, Y. S.; Zhang, M.; Ma, X. D.; Liu, Z. W.; Luo, J. H.; Lyu, C.; Wang, H. L.; Ma, J.; Zhao, Y. L.; Zhou, F. J.; Huang, Y.; Xie, D.; Yang, Y. G., 5-methylcytosine promotes pathogenesis of bladder cancer through stabilizing mRNAs. Nat Cell Biol 2019, 21 (8), 978-990.
12. Chen, P.; Crain, P. F.; Nasvall, S. J.; Pomerantz, S. C.; Bjork, G. R., A “gain of function” mutation in a protein mediates production of novel modified nucleosides. EMBO J 2005, 24 (10), 1842-51.
13. Zhang, L. S.; Xiong, Q. P.; Pena Perez, S.; Liu, C.; Wei, J.; Le, C.; Zhang, L.; Harada, B. T.; Dai, Q.; Feng, X.; Hao, Z.; Wang, Y.; Dong, X.; Hu, L.; Wang, E. D.; Pan, T.; Klungland, A.; Liu, R. J.; He, C., ALKBH7-mediated demethylation regulates mitochondrial polycistronic RNA processing. Nat Cell Biol 2021, 23 (7), 684-691.
14. Wang, K.; Yi, C., Coupling Transcription and Translation via the Epitranscriptomic m(6)A Mark. Biochemistry 2019, 58 (5), 297-298.
15. Wang, Y.; Zhang, X.; Zou, G.; Peng, S.; Liu, C.; Zhou, X., Detection and Application of 5-Formylcytosine and 5-Formyluracil in DNA. Acc Chem Res 2019, 52 (4), 1016-1024.
16. Song, J.; Zhuang, Y.; Zhu, C.; Meng, H.; Lu, B.; Xie, B.; Peng, J.; Li, M.; Yi, C., Differential roles of human PUS10 in miRNA processing and tRNA pseudouridylation. Nat Chem Biol 2020, 16 (2), 160-169.
17. Chen, Y. S.; Yang, W. L.; Zhao, Y. L.; Yang, Y. G., Dynamic transcriptomic m(5)C and its regulatory role in RNA processing. Wiley Interdiscip Rev RNA 2021, 12 (4), e1639.
18. Grozhik, A. V.; Jaffrey, S. R., Epitranscriptomics: Shrinking maps of RNA modifications. Nature 2017, 551 (7679), 174-176.
19. Patil, D. P.; Pickering, B. F.; Jaffrey, S. R., Reading m(6)A in the Transcriptome: m(6)A-Binding Proteins. Trends Cell Biol 2018, 28 (2), 113-127.
20. Meyer, K. D.; Jaffrey, S. R., Rethinking m(6)A Readers, Writers, and Erasers. Annu Rev Cell Dev Biol 2017, 33, 319-342.
21. Yu, Q.; Liu, S.; Yu, L.; Xiao, Y.; Zhang, S.; Wang, X.; Xu, Y.; Yu, H.; Li, Y.; Yang, J.; Tang, J.; Duan, H. C.; Wei, L. H.; Zhang, H.; Wei, J.; Tang, Q.; Wang, C.; Zhang, W.; Wang, Y.; Song, P.; Lu, Q.; Zhang, W.; Dong, S.; Song, B.; He, C.; Jia, G., RNA demethylation increases the yield and biomass of rice and potato plants in field trials. Nat Biotechnol 2021, 39 (12), 1581-1588.
22. Dumelin, C. E.; Chen, Y.; Leconte, A. M.; Chen, Y. G.; Liu, D. R., Discovery and biological characterization of geranylated RNA in bacteria. Nat Chem Biol 2012, 8 (11), 913-9.
23. Sierant, M.; Leszczynska, G.; Sadowska, K.; Dziergowska, A.; Rozanski, M.; Sochacka, E.; Nawrot, B., S-Geranyl-2-thiouridine wobble nucleosides of bacterial tRNAs; chemical and enzymatic synthesis of S-geranylated-RNAs and their physicochemical characterization. Nucleic Acids Res 2016, 44 (22), 10986-10998.
24. Sierant, M.; Leszczynska, G.; Sadowska, K.; Komar, P.; Radzikowska-Cieciura, E.; Sochacka, E.; Nawrot, B., Escherichia coli tRNA 2-selenouridine synthase (SelU) converts S2U-RNA to Se2U-RNA via S-geranylated-intermediate. FEBS Lett 2018, 592 (13), 2248-2258.
25. Wei, G.; Eberl, F.; Chen, X.; Zhang, C.; Unsicker, S. B.; Kollner, T. G.; Gershenzon, J.; Chen, F., Evolution of isoprenyl diphosphate synthase-like terpene synthases in fungi. Sci Rep 2020, 10 (1), 14944.
26. Wang, R.; Ranganathan, S. V.; Basanta-Sanchez, M.; Shen, F.; Chen, A.; Sheng, J., Synthesis and base pairing studies of geranylated 2-thiothymidine, a natural variant of thymidine. Chem Commun (Camb) 2015, 51 (91), 16369-72.
27. Wang, R.; Vangaveti, S.; Ranganathan, S. V.; Basanta-Sanchez, M.; Haruehanroengra, P.; Chen, A.; Sheng, J., Synthesis, base pairing and structure studies of geranylated RNA. Nucleic Acids Res 2016, 44 (13), 6036-45.
28. Rilling, H. C.; Breunger, E.; Epstein, W. W.; Crain, P. F., Prenylated proteins: the structure of the isoprenoid group. Science 1990, 247 (4940), 318-20.
29. Gershenzon, J.; Dudareva, N., The function of terpene natural products in the natural world. Nat Chem Biol 2007, 3 (7), 408-14.
30. Jha, N. K.; Sharma, C.; Hashiesh, H. M.; Arunachalam, S.; Meeran, M. N.; Javed, H.; Patil, C. R.; Goyal, S. N.; Ojha, S., beta-Caryophyllene, A Natural Dietary CB2 Receptor Selective Cannabinoid can be a Candidate to Target the Trinity of Infection, Immunity, and Inflammation in COVID-19. Front Pharmacol 2021, 12, 590201.
31. Baranyi, M.; Buday, L.; Hegedus, B., K-Ras prenylation as a potential anticancer target. Cancer Metastasis Rev 2020, 39 (4), 1127-1141.
32. Li, F.; Dong, J.; Hu, X.; Gong, W.; Li, J.; Shen, J.; Tian, H.; Wang, J., A covalent approach for site-specific RNA labeling in Mammalian cells. Angew Chem Int Ed Engl 2015, 54 (15), 4597-602.
33. Jingwen Zhang, J. Z., Li Wang, Hongmei Hu, Sheng Wang, Ping Yu, Rui Wang, Labeling of polyprenylated macromolecules using mild Ene reactions. Tetrahedron 2021, 81.
34. Alexander, S. C.; Busby, K. N.; Cole, C. M.; Zhou, C. Y.; Devaraj, N. K., Site-Specific Covalent Labeling of RNA by Enzymatic Transglycosylation. J Am Chem Soc 2015, 137 (40), 12756-9.
35. Wang, S.; Li, Y.; Zhou, H.; Wang, L.; Wang, R., Development of Biocompatible Ene-Ligation Enabled by Prenyl-Based beta-Caryophyllene with Triazoline/Selectfluor under Physiological Conditions. J Org Chem 2022, 87 (13), 8648-8655.
36. Jingwen Zhang, Y. L., Sheng Wang, Rui Wang, Labeling of prenylated nucleic acid by Ene-type fluorination under physiological condition. Tetrahedron Letters 2021, 74.
37. Jager, G.; Chen, P.; Bjork, G. R., Transfer RNA Bound to MnmH Protein Is Enriched with Geranylated tRNA—A Possible Intermediate in Its Selenation? PLoS One 2016, 11 (4), e0153488.
38. Walker, J. E.; Saraste, M.; Runswick, M. J.; Gay, N. J., Distantly related sequences in the alpha- and beta-subunits of ATP synthase, myosin, kinases and other ATP-requiring enzymes and a common nucleotide binding fold. EMBO J 1982, 1 (8), 945-51.
39. Koonin, E. V., A superfamily of ATPases with diverse functions containing either classical or deviant ATP-binding motif. J Mol Biol 1993, 229 (4), 1165-74.
40. Patil, A.; Dyavaiah, M.; Joseph, F.; Rooney, J. P.; Chan, C. T.; Dedon, P. C.; Begley, T. J., Increased tRNA modification and gene-specific codon usage regulate cell cycle progression during the DNA damage response. Cell Cycle 2012, 11 (19), 3656-65.
41. Begley, U.; Sosa, M. S.; Avivar-Valderas, A.; Patil, A.; Endres, L.; Estrada, Y.; Chan, C. T.; Su, D.; Dedon, P. C.; Aguirre-Ghiso, J. A.; Begley, T., A human tRNA methyltransferase 9-like protein prevents tumour growth by regulating LIN9 and HIF1-alpha. EMBO Mol Med 2013, 5 (3), 366-83.
42. Rapino, F.; Delaunay, S.; Rambow, F.; Zhou, Z.; Tharun, L.; De Tullio, P.; Sin, O.; Shostak, K.; Schmitz, S.; Piepers, J.; Ghesquiere, B.; Karim, L.; Charloteaux, B.; Jamart, D.; Florin, A.; Lambert, C.; Rorive, A.; Jerusalem, G.; Leucci, E.; Dewaele, M.; Vooijs, M.; Leidel, S. A.; Georges, M.; Voz, M.; Peers, B.; Buttner, R.; Marine, J. C.; Chariot, A.; Close, P., Codon-specific translation reprogramming promotes resistance to targeted therapy. Nature 2018, 558 (7711), 605-609.
43. Chavent, M.; Duncan, A. L.; Rassam, P.; Birkholz, O.; Helie, J.; Reddy, T.; Beliaev, D.; Hambly, B.; Piehler, J.; Kleanthous, C.; Sansom, M. S. P., How nanoscale protein interactions determine the mesoscale dynamic organisation of bacterial outer membrane proteins. Nat Commun 2018, 9 (1), 2846.
44. Wickenhagen, A.; Sugrue, E.; Lytras, S.; Kuchi, S.; Noerenberg, M.; Turnbull, M. L.; Loney, C.; Herder, V.; Allan, J.; Jarmson, I.; Cameron-Ruiz, N.; Varjak, M.; Pinto, R. M.; Lee, J. Y.; Iselin, L.; Palmalux, N.; Stewart, D. G.; Swingler, S.; Greenwood, E. J. D.; Crozier, T. W. M.; Gu, Q.; Davies, E. L.; Clohisey, S.; Wang, B.; Trindade Maranhao Costa, F.; Freire Santana, M.; de Lima Ferreira, L. C.; Murphy, L.; Fawkes, A.; Meynert, A.; Grimes, G.; Investigators, I. C.; Da Silva Filho, J. L.; Marti, M.; Hughes, J.; Stanton, R. J.; Wang, E. C. Y.; Ho, A.; Davis, I.; Jarrett, R. F.; Castello, A.; Robertson, D. L.; Semple, M. G.; Openshaw, P. J. M.; Palmarini, M.; Lehner, P. J.; Baillie, J. K.; Rihn, S. J.; Wilson, S. J., A prenylated dsRNA sensor protects against severe COVID-19. Science 2021, 374 (6567), eabj3624.
45. Kmiec, D.; Lista, M. J.; Ficarelli, M.; Swanson, C. M.; Neil, S. J. D., S-farnesylation is essential for antiviral activity of the long ZAP isoform against RNA viruses with diverse replication strategies. PLoS Pathog 2021, 17 (10), e1009726.

### Example II: Fluorescent Labeling and Iodine-Mediated Cyclization of i⁶A

### 1. Materials

All chemicals were purchased from Sigma-Aldrich, Acros, Innochem, Macklin Inc, Energy Chemical and were used without further purification. Extra dry solvents, such as 1,4-dioxane, DMF, and THF, were obtained from Innochem in sealed bottles over 3 or 4 Å molecular sieves and stored under dried nitrogen. Organic solvents were obtained from Sinopharm Chemical Reagent Co., Ltd (Shanghai, China) and used in reactions, column chromatography and recrystallizations. Milli-Q ultrapure water (resistivity, 18 m (2) purified through Millipore Milli-Q Advantage A1 purification system was used for all bioconjugation reactions. The reactions were monitored by thin-layer chromatography (TLC) analysis using silica gel (60-Å pore size, F254, Yantai Chemical Industry Research Institute) plates. Compounds were visualized by UV irradiation (λ=254 nm) and/or spraying TLC stain such as a KMnO₄ solution followed by electronic heating. Flash chromatography columns were performed on silica gel (60-Å pore size, 230-400 mesh). HPLC purification were performed using EasyChrom-1000 system with NU3000 serials UV/Vis. detector (Hanbon Sci. & Tech., Jiangsu, China) using an Ultimate® XB-C₁₈ column, 21.2× 250 mm 5 micron (Welch Materials Inc., Shanghai, China). Separation was achieved by gradient elution from 5% to 70% acetonitrile in water (constant 0.1% formic acid) over 20 min, isocratic elution with 70% acetonitrile from 20 to 25 min, and returned to initial conditions and equilibrated for 5 min. The LC chromatograms were recorded by monitoring absorption at 254 nm and 220 nm. ¹H and ¹³C NMR spectra were recorded at room temperature on a Bruker spectrometer (AM-600 or AM-400) operating at 600/400 and 150/100 MHz, respectively. Chemical shifts are given in parts per million, and 1H and 13C {1H} NMR spectra were referenced using the solvent signal as an internal standard. The following abbreviations are used for the proton spectra multiplicities: s: singlet, d: doublet, t: triplet, m: multiplet, br: broad. Coupling constants (J) are reported in Hertz (Hz). HRMS (TOF) were obtained from the Bruker Micro TOF II Spectrometer using Electro Spray Ionization (ESI). MS (ESI) was obtained from the Expression L (Beijing Bohui Innovation Biotechnology Co., Ltd). LC-MS analysis was obtained from the Bruker Orbitrap LC/MS (Q Exactive™) at Huazhong University of Science and Technology Analytical and Testing Center. UV-visible absorbance measurements were performed UV-visible spectrometer (Lambda365, PerkinElmer, German). Confocal Imaging was performed using a LSM 780 confocal microscope (Zeiss) with a 20× objective at 16-bit depth under non-saturating conditions. EGFP were imaged with a 480 nm (excitation) and a 510 nm (emission) and false-colored green.

TABLE 2
Materials.

| Entries | Name | Vendor | Product# |
|---|---|---|---|
| 1 | T7 RNA Polymerase | Thermo Fisher Scientific | #18033100 |
| 2 | DNase I | Invitrogen ™ (Thermo Fisher | #18047019 |
| | | Scientific) | |
| 3 | Transcript Aid T7 High Yield | Thermo Fisher Scientific | #K0441 |
| | Transcription Kit | | |
| 4 | Glycogen | Thermo Fisher Scientific | #R0561 |
| 5 | Pyrobest ™ DNA | TaKaRa Shuzo Co. Ltd. | #R005Q |
| | Polymerase | (Tokyo, Japan) | |
| 6 | PrimeSTAR HS DNA | TaKaRa Shuzo Co. Ltd. | #R010Q |
| | Polymerase | (Tokyo, Japan) | |
| 7 | DNA Clean & | Zymo Research Corp | #D4014 |
| | Concentrator ™-5 Kit | | |
| 8 | DNeasy Blood & Tissue | QIAGEN | #69504 |
| | Kit | | |
| 9 | DNA Size Marker | TsingKe Biotech (Beijing, | #TSJ102 |
| | | China) | |
| 10 | rNTP Mixture | Sangon Biotech (Shanghai, | #B600057 |
| | | China) | |
| 11 | Nucleic Acid Stains Super | US Everbright Inc. (Suzhou, | #S-2001 |
| | GelRed | China) | |
| 12 | GoldView ™ Nucleic Acid | Yeasen Biotech (Shanghai, | #10201ES03 |
| | Dye Staining | China) | |
| 13 | RIPA | Sangon Biotech (Shanghai, | #C500005 |
| | | China) | |
| 14 | DMEM Medium | Gibco ™ (Thermo Fisher | #11960077 |
| | | Scientific) | |
| 15 | 10% Fetal Bovine Serum | Gibco ™ (Thermo Fisher | #26010066 |
| | (FBS) | Scientific) | |
| 16 | Penicillin-Streptomycin | Gibco ™ (Thermo Fisher | #10378016 |
| | | Scientific) | |
| 17 | Mth RNA ligase | NEB | #M2611A |
| 18 | Alcohol | Innochem (Beijing, China) | #G00021 |
| 19 | RNase free DEPC water | Innochem (Beijing, China) | #B46770 |
| 20 | High-Capacity cDNA | Applied Biosystems ™ (Thermo | #4368814 |
| | Reverse Transcription Kit | Fisher Scientific) | |
| 21 | T-Vector pMD ™19 | TaKaRa Shuzo Co. Ltd. | #3271 |
| | | (Tokyo, Japan) | |
| 22 | DH5α E. coli. | TaKaRa Shuzo Co. Ltd. | #9057 |
| | | (Tokyo, Japan) | |
| 23 | GAPDH | Invitrogen ™ (Thermo Fisher | #PA1-987 |
| | | Scientific) | |
| 24 | Notch1 | Invitrogen ™ (Thermo Fisher | #41-3500 |
| | | Scientific) | |
| 25 | HeLa cells | ATCC | #30-2020 |
| 26 | CyQUANT ™ MTT | Invitrogen ™ (Thermo Fisher | V13154 |
| | | Scientific) | |
| 27 | Total RNA Extraction Kit | Invitrogen ™ (Thermo Fisher | #15596026 |
| | | Scientific) | |
| 28 | Total tRNA (E. coli | Merck | #10109541001 |
| | MRE600) | | |
| 29 | N-Bromosuccinimide | Innochem (Beijing, China) | #A02193 |
| 30 | Iodine | Thermo Fisher Scientific | #35634 |
| 31 | Dimethyl sulfoxide | Thermo Fisher Scientific | #D159-4 |
| 32 | Sodiumthiosulfate | Innochem (Beijing, China) | #A44684 |
| 33 | Sodium Carbonate | Innochem (Beijing, China) | #A19943 |
| 34 | Acetonitrile | Innochem (Beijing, China) | #A0080 |

### 2. Preliminary Rearrangement Investigations

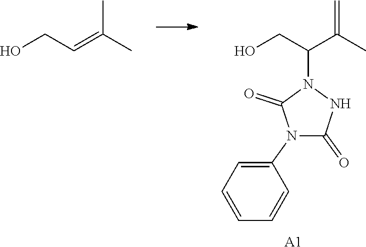

To a stirred solution of prenyl alcohol (8.6 mg, 0.1 mmol) was added 4-phenyl-3H-1, 2, 4-triazole-3, 5 (4H)-dione (PTAD, 17 mg, 0.1 mmol) in acetonitrile (0.6 mL). The mixture continued for 12 hours. Solvent was removed and the crude product was purified by flash chromatography (eluent: PE:ethyl acetate=10:1) to afford yellowish oil product A1 (20 mg, 0.077 mmol) in 82% yield.

¹H NMR (400 MHz, CDCl₃) δ 7.50-7.25 (m, 5H), 5.02 (s, 1H), 4.92 (s, 1H), 4.64-4.61 (m, 1H), 4.00-3.95 (m, 1H), 3.88-3.84 (m, 1H), 1.75 (s, 3H). MS (ESI) Calculated for [M+H]⁺=262.1. found 262.0.

### 3. Synthesis Nucleoside i⁶A (4)

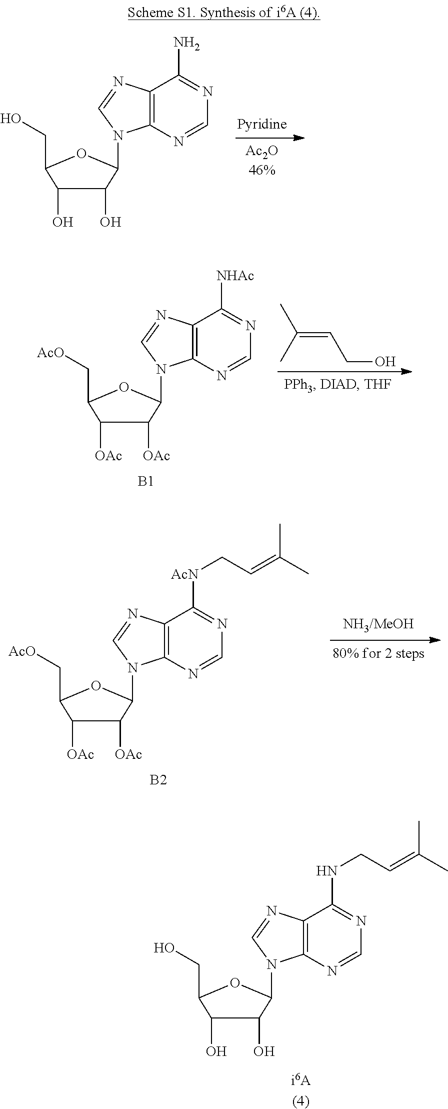

### Synthesis of N⁶-acetyl-2′, 3′, 5′-tri-O-acetyladenosine (B1)

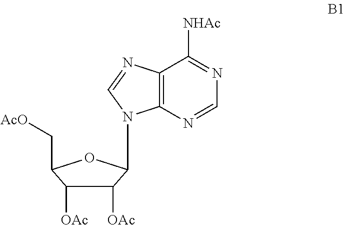 [SMILES: CC(=O)Nc1ncnc2c1ncn2C1OC(COC(C)=O)C(OC(C)=O)C1OC(C)=O]

A mixture of adenosine (2.0 g, 7.48 mmol), pyridine (15 mL), and Ac₂O (7 mL, 74.2 mmol) was stirred at room temperature overnight. The resulting clear solution was refluxed at 60° C. overnight. The reaction was cooled down and quenched by EtOH. Then the reaction mixture was co-evaporated with addition of excess of EtOH to remove pyridine completely. The resultant foam was dissolved in MeOH (20 mL) and imidazole (0.4 g, 5.88 mmol) was added and the solution was stirred at room temperature. After 8 hours, the solution was diluted with ethyl acetate (50 mL) and washed by brine (5×50 mL). Then the organic solvent was dried over Na₂SO₄, filtered and concentrated. The residue was purified by silica gel (CH₂Cl₂:MeOH=30:1) to give B1 (1.48 g, 3.4 mmol, 46%) as yellowish solid. ¹H NMR (400 MHz, CDCl₃) δ 9.16 (s, 1H), 8.70 (s, 1H), 8.23 (s, 1H), 6.23 (d, J=4.0 Hz, 1H), 5.97 (t, J=8.0 Hz, 1H), 5.68 (t, J=12.0 Hz, 1H), 4.48-4.44 (m, 2H), 4.41-4.36 (m, 1H), 2.63 (s, 3H), 2.16 (s, 3H), 2.12 (s, 3H), 2.09 (s, 3H). ¹³C NMR (101 MHz, CDCl₃) δ 170.80, 170.36, 169.60, 169.39, 152.66, 151.04, 149.52, 141.47, 122.23, 86.45, 80.40, 73.09, 70.61, 63.03, 25.75, 20.75, 20.54, 20.39. MS (ESI) Calculated for [M+Na]⁺=458.1. found 458.0.

### Synthesis of N⁶-acetyl-2′, 3′, 5′-tri-O-acetyl-N⁶-(3-methylbut-2-enyl) adenosine (B2)

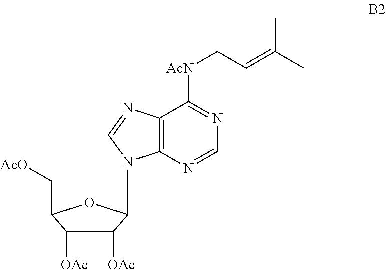

A mixture of B1 (1.3 g, 3 mmol), triphenylphosphine (PPh₃, 1.2 g, 4.5 mmol), and 3-methyl-2-buten-1-ol (387 mg, 4.5 mmol) in THF (5.0 mL) was stirred at r.t. until a homogeneous solution was formed. Di-isopropyl azodicarboxylate (DIAD, 0.9 g, 4.5 mmol) was added in one portion. The reaction was monitored by TLC. A second addition of triphenylphosphine (4.5 mmol), alcohol and DIAD was made to achieve complete conversion of starting material B1 after 20 hours. After 5 hours the mixture was evaporated and the residue was purified by column chromatography (CH₂Cl₂:MeOH=50:1) to give crude product B2 (2.33 g, 4.63 mmol) as white solid, which was taken to the next step directly

### Synthesis of N⁶-(3-methylbut-2-enyl)-adenosine (4, i⁶A)

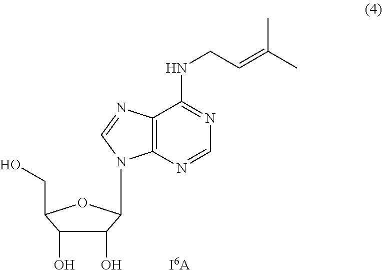 [SMILES: CC(C)=CCNc1ncnc2c1ncn2C1OC(CO)C(O)C1O ; "I6A"]

Compound B2 (2.33 g, 4.63 mmol) was dissolved in 7.0 M NH₃ in MeOH solution (1.0 mL, 7.0 mmol) and the solution was stirred for 48 hours. The volatiles were evaporated under vacuo and the residue was purified by silica gel chromatography (PE:ethyl acetate=1:1) to give nucleoside i⁶A (4, 800 mg, 2.38 mmol, 80% over 2 steps) as white solid.

¹H NMR (400 MHz, DMSO-d₆) δ 8.35 (s, 1H), 8.22 (s, 1H), 7.91 (s, 1H), 5.89 (d, J=4.0 Hz, 1H), 5.45-5.42 (m, 2H), 5.31 (t, J=16.0 Hz, 1H), 5.19 (d, J=8.0 Hz, 1H), 4.62 (q, J=16.0 Hz, 1H), 4.16 (q, J=12.0 Hz, 1H), 3.98 (q, J=8.0 Hz, 1H), 3.71-3.66 (m, 1H), 3.59-3.53 (m, 1H), 1.69 (d, J=12.0 Hz, 6H). ¹³C NMR (101 MHz, DMSO-d₆) δ 172.17, 154.80, 152.79, 140.14, 133.81, 122.43, 120.29, 88.45, 86.38, 73.97, 71.13, 62.14, 38.13, 25.84, 22.91, 18.29. HRMS (ESI/Q-TOF): m/z: [M+H]⁺ Calculated for C₁₅H₂₂N₅O₄ 336.1672. Found 336.1690.

### 4. Synthesis of Fluorescent Probes

### Probe PTAD-DBCO-FITC (8).

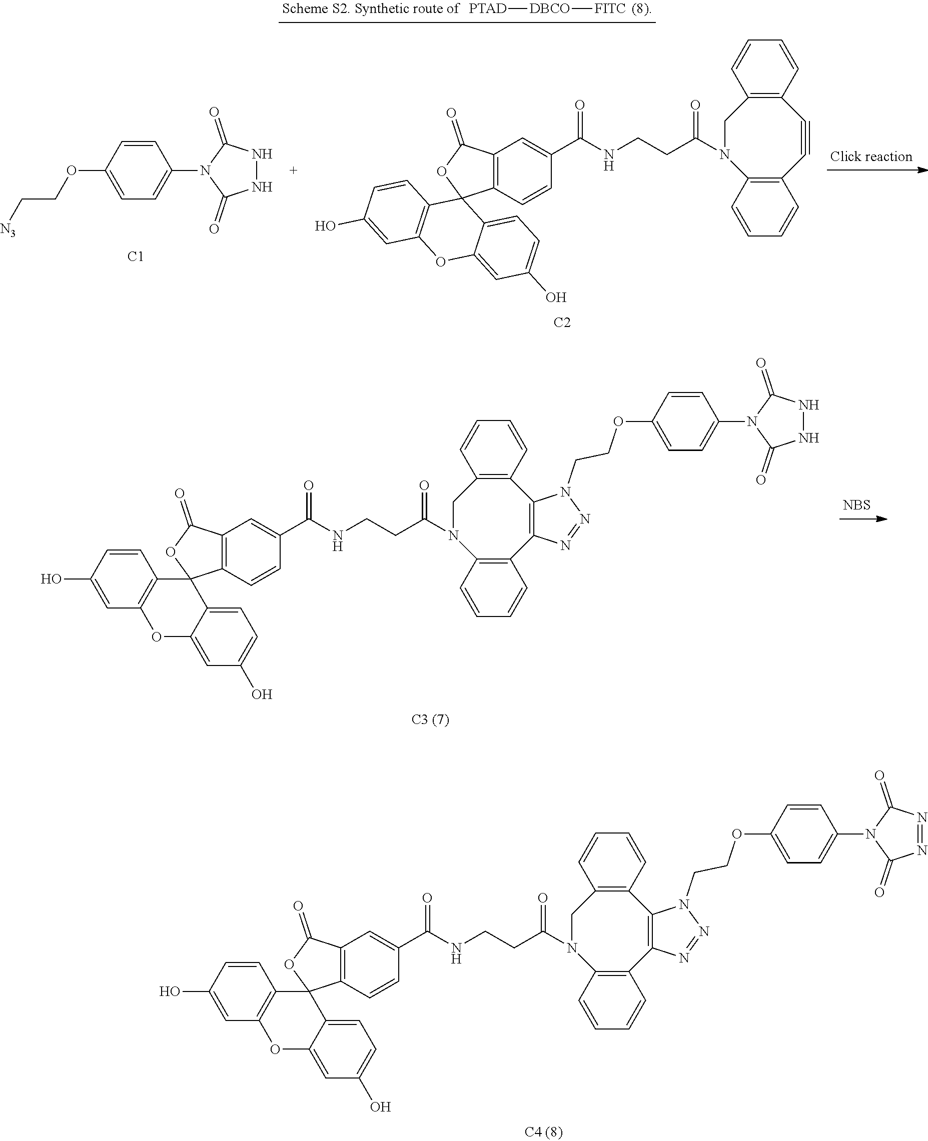

A solution of compound C1 (10 mg, 0.04 mmol) and FITC-DBCO (C2, 5 mg, 0.01 mmol) in dry DMF (1.0 mL) under nitrogen was stirred at room temperature overnight. The reaction mixture was purified by RP-HPLC (separation was achieved using an Ultimate XB-C₁₈ column, 21.2× 250 mm 5 micron (Welch Materials Inc., Shanghai, China) by gradient elution from 5% to 70% acetonitrile in water (constant 0.1% formic acid) over 15 min, isocratic elution with 70% acetonitrile from 15 to 30 minutes, and returned to initial conditions and equilibrated for 5 minutes to give PTAD-DBCO-FITC (7, 1.0 mg, 0.001 mmol, 11%).

¹H NMR (400 MHz, methanol-d₄) δ 8.23 (s, 1H), 7.99 (d, J=8.0 Hz, 1H), 7.54 (d, J=8.0 Hz, 2H), 7.45 (s, 2H), 7.29 (d, J=4.0 Hz, 1H), 7.25-7.23 (m, 3H), 7.29 (t, J=8.0 Hz, 2H), 7.15 (d, J=8.0 Hz, 1H), 6.95 (d, J=8.0 Hz, 1H), 6.86 (d, J=8.0 Hz, 1H), 6.60 (s, 2H), 6.51 (q, J=12.0 Hz, 2H), 6.46-6.42 (m, 2H), 5.94 (d, J=20.0 Hz, 1H), 4.42 (t, J=16.0 Hz, 1H), 4.36 (t, J=8.0 Hz, 1H), 3.40-3.30 (m, 2H), 2.24-2.08 (m, 2H), 1.96-1.74 (m, 2H). MS (ESI) Calculated for [M+H]⁺=897.3. found 897.2. HRMS (ESI/Q-TOF): m/z: [M+H]⁺ Calculated for C₄₉H₃₇N₈O₁₀ 897.2633. found 897.2616; HRMS (ESI/Q-TOF): m/z: [M+Na]⁺ Calculated for C₄₉H₃₆N₈NaO₁₀ 919.2452. found 919.2436. PTAD-DBCO-FITC (8) was obtained by in situ oxidation using NBS (DMF solution) at room temperature for 5 minutes.

### Probe PTAD-DBCO-Cy5 (10).

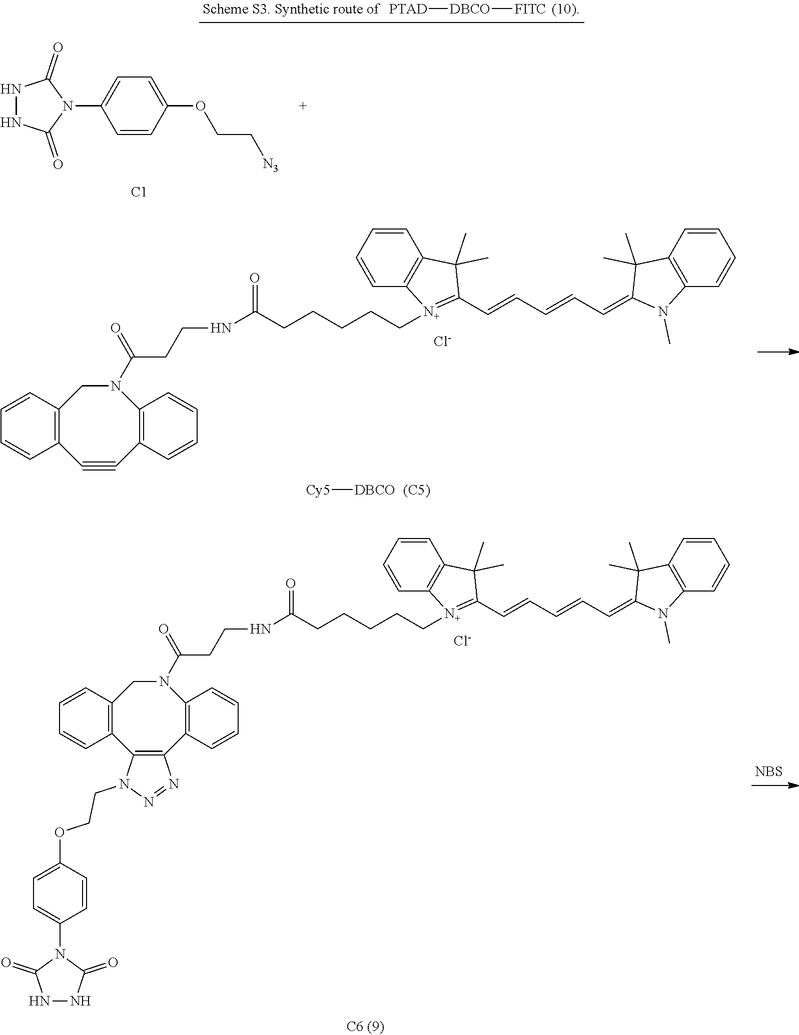

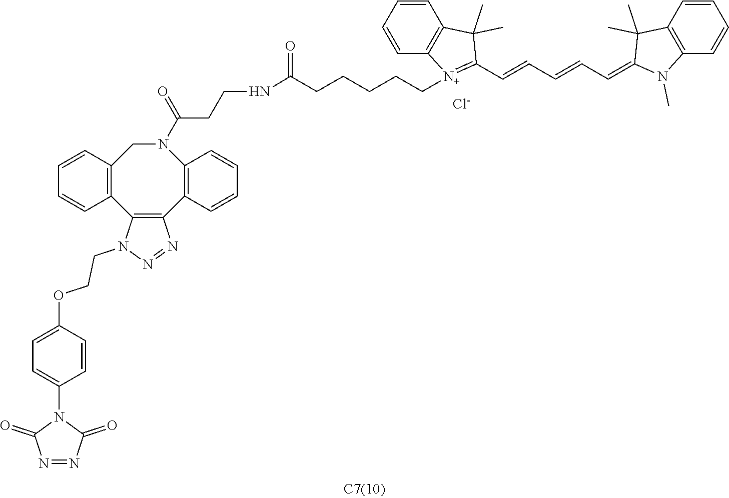 [SMILES: CN1/C(=C/C=C/C=C/C2=[N+](CCCCCC(=O)NCCC(=O)N3Cc4ccccc4-c4c(nnn4CCOc4ccc(N5C(=O)N=NC5=O)cc4)-c4ccccc43)c3ccccc3C2(C)C)C(C)(C)c2ccccc21 ; [Cl-]]

A solution of compound C1 (1.7 mg, 0.0064 mmol) and Cy5-DBCO (C5, 2.5 mg, 0.0032 mmol) in dry CH₃CN (1.0 mL) under nitrogen was stirred at room temperature overnight. The reaction mixture was purified by RP-HPLC (Separation was achieved using a Ultimate XB-C₁₈ column, 21.2×250 mm 5 micron (Welch Materials Inc., Shanghai, China) by gradient elution from 5% to 70% acetonitrile in water (constant 0.1% formic acid) over 15 minutes, isocratic elution with 70% acetonitrile from 15 to 30 minutes, and returned to initial conditions and equilibrated for 5 minutes) to give compound 9 (3.0 mg, 0.0028 mmol, 90%) as blue solid, which was dissolved in anhydrous DMF (0.14 mL) and was treated equal equivalent N-bromo succinimide (NBS) solution (20 mM in DMF) to prepare PTAD-DBCO-Cy5 stock solution (10, 10 mM in DMF, stored in −20° C.). MS (ESI) Calculated for [M−H]⁻=1035.5. found 1035.8. HRMS (ESI/Q-TOF): m/z: [M-Cl]⁺, Calculated for C₆₀H₆₃N₁₀O₅⁺ 1003.4977. found 1003.4964 {for PTAD-DBCO-Cy5 precursor (9)}.

### 5. Investigations of Reaction Properties of i⁶A (4) with PTAD (5)

Reaction of i⁶A (4) with PTAD (5).

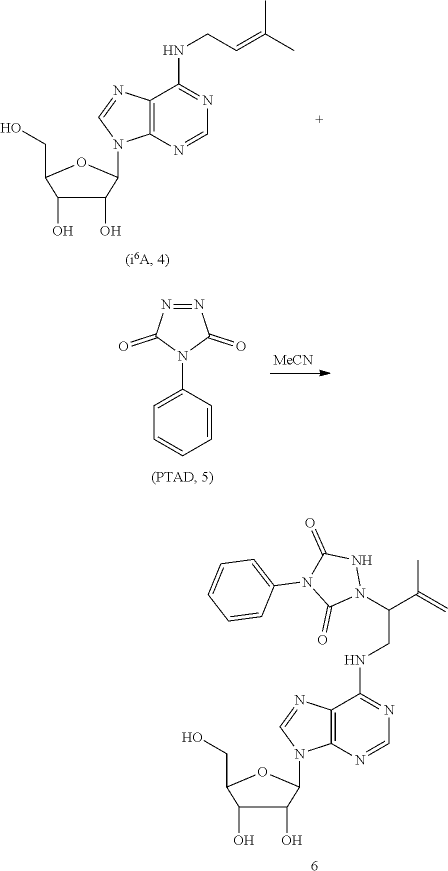

To a solution of nucleoside i⁶A (4, 33 mg, 0.1 mmol) in acetonitrile (1.0 mL) was added 4-phenyl-1, 2, 4-triazoline-3, 5-dione (5, PTAD, 34 mg, 0.2 mmol) and stirred at room temperature until the red color of PTAD disappeared. Then the organic solvents were removed. The residue was purified by silica gel column chromatography (CH₂Cl₂:MeOH=10:1) to afford adduct 6 (20 mg, 0.039 mmol, 39%) as white solid.

¹H NMR (400 MHz, methanol-d₄) δ 8.26 (t, J=12.0 Hz, 2H), 7.51 (d, J=4.0 Hz, 1H), 7.42-7.33 (m, 4H), 7.24-7.19 (m, 2H), 5.95 (q, J=8.0 Hz, 1H), 5.18 (d, J=12.0 Hz, 2H), 5.07 (t, J=16.0 Hz, 1H), 4.76-4.70 (m, 1H), 4.32 (q, J=8.0 Hz, 1H), 4.18-4.11 (m, 3H), 3.88 (q, J=16.0 Hz, 1H), 3.76-3.72 (m, 1H), 1.90 (s, 3H). ¹³C NMR (151 MHz, methanol-d₄) δ 154.92, 153.72, 152.02, 140.41, 131.48, 128.75, 128.59, 128.58, 127.86, 126.03, 124.44, 120.04, 113.87, 89.95 (d, J=21.1 Hz), 86.73, 74.06 (d, J=27.2 Hz), 71.29 (d, J=18.1 Hz), 62.10 (d, J=15.1 Hz), 59.06, 48.18, 20.15 (d, J=4.5 Hz). MS (ESI) Calculated for [M+Na]⁺=533.2. found=533.0. HRMS (ESI/Q-TOF): m/z: [M+H]⁺, Calculated for C₂₃H₂₇N₈O₆ 511.2054. found 511.2041.

Dynamic Investigations of i⁶A (4) with PTAD (5).

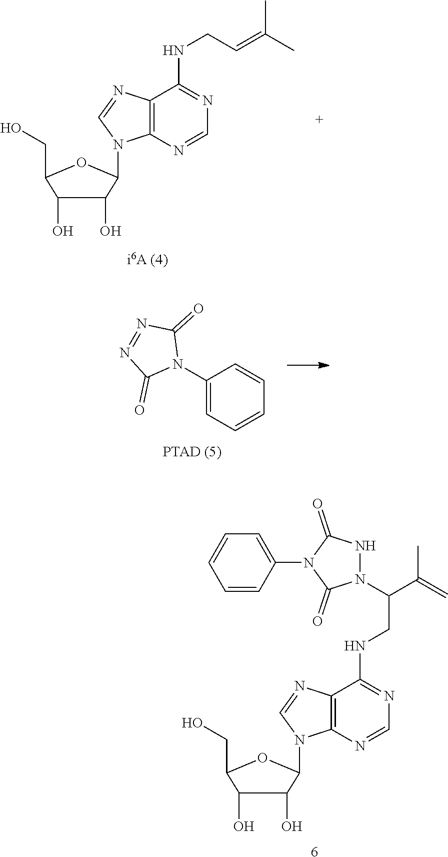

Nucleoside i⁶A (4, 10 mg, 0.03 mmol) were dissolved in CD₃CN/D₂O (1.0 mL, v/v=1:1, final concentration=30 mM). PTAD (5, 26 mg, 0.15 mmol) was then added to the solution (final concentration=150 mM), and the reaction mixture was allowed to stand at room temperature. The ¹H NMR spectra were recorded by proton NMR (400 MHz).

### 6. Synthesis of Nucleotide i⁶ATP

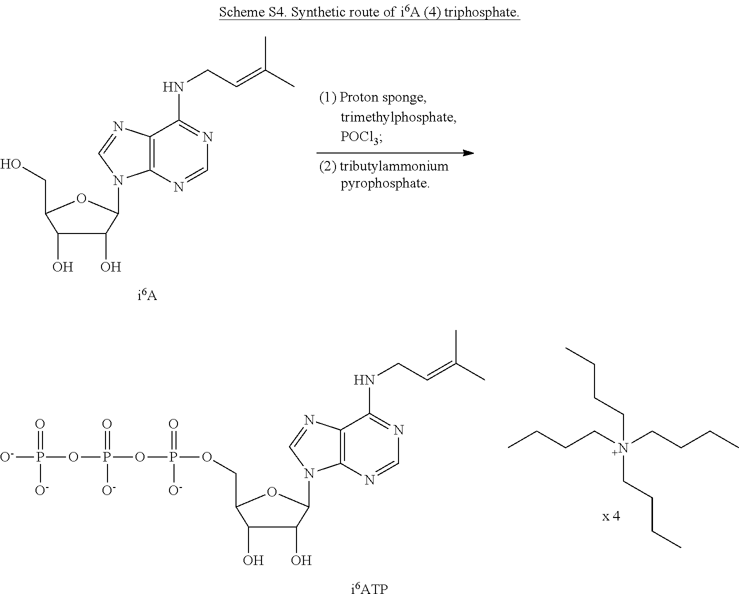 [SMILES: CC(C)=CCNc1ncnc2c1ncn2C1OC(CO)C(O)C1O ; "i6A" ; CC(C)=CCNc1ncnc2c1ncn2C1OC(COP(=O)([O-])OP(=O)([O-])OP(=O)([O-])[O-])C(O)C1O ; CCCC[N+](CCCC)(CCCC)CCCC ; "i6ATP"]

Nucleoside i⁶A (4, 35 mg, 0.1 mmol) and proton sponge (58 mg, 0.25 mmol) were dissolved in trimethyl phosphate (0.7 mL) and was placed under the ice-water bath. Phosphorus oxychloride (POCl₃, 33.6 μL, 0.36 mmol) was slowly added and stirred at 0° C. for 12 hours. A solution of tributylamine (175 μL, 0.72 mmol) and tributyl ammonium pyrophosphate (642 mg) in DMF (2.0 mL) was slowly added and the reaction mixture was stirred at 0° C. for 30 minutes. The reaction was quenched by addition of 1.0 M aqueous triethylammonium bicarbonate (TEAB, pH=7.5, 15 mL). The mixture was diluted with H₂O (5.0 mL) and subjected to HPLC purification. Separation was achieved using an Ultimate XB-C₁₈ column, 21.2×250 mm 5 micron by gradient elution from 5% to 50% acetonitrile in water (constant 0.1% formic acid) over 25 minutes, isocratic elution with 50% acetonitrile from 25 to 30 minutes, and returned to initial conditions and equilibrated for 5 minutes to give i⁶ATP (10.0 mg, as tributyl ammonium salt).

¹H NMR (400 MHz, methanol-d₄) δ 8.40 (s, 2H), 8.30 (s, 1H), 6.13 (d, J=4.0 Hz, 1H), 5.40 (t, J=12.0 Hz, 2H), 4.73 (q, J=16.0 Hz, 1H), 4.57-4.43 (m, 1H), 4.27-4.26 (m, 2H), 4.20-4.13 (m, 2H). ³¹P NMR (162 MHz, methanol-d₄) δ −10.47 (d, J=22.68 Hz, α-P), −11.46 (d, J=22.68 Hz, γ-P), −24.00 (t, J=22.68 Hz, β-P). MS (ESI) Calculated for [M−H]⁻=574.1. found 574.2.

### 7. Profiling of i⁶A-Incorporated RNA

7.1 Test the Ability of T7 RNA Polymerase to Recognize Modified Nucleotides (i⁶ATP) at Specific Sites.

### Step 1. Construction of T7 RNA Polymerase-Mediated EGFP Transcription System In Vitro.

In view of the high specificity of T7 RNA polymerase, T7 promoter as a strong promoter can efficiently guide the expression of downstream genes. Here, the T7 promoter sequence is used to guide the transcription of EGFP in vitro.

EGFP gene sequence was amplified from pEGFP-C1 plasmids. Then T7 promoter sequence was linked to the 5′-end by fusion PCR. The primers are summarized as follow:

TABLE 3
Sequences for the construction
of chimeric T7P-EGFP.

| Name | Sequence (5′-3′) |
|---|---|
| T7P-GFP-F | TAATACGACTCACTATAGGGATGGTGAGCAAGGGCGAGG |
| | AGCTG SEQ ID NO: 7 |
| T7P-GFP-R | CTACTTGTACAGCTCGTCCATGCCG SEQ ID NO: 8 |

### Step 2. Detection of T7 RNA Polymerase Catalyzed RNA Polymerization Process In Vitro.

Using the T7P-EGFP constructed above as a template and rNTP as substrates, the reaction was carried out at 37° C. for 2 hours which was catalyzed by T7 RNA polymerase. After the reaction, the template DNA T7P-EGFP was degraded with DNase I. Finally, the RNA was precipitated and purified, and the transcription effect of the RNA was detected by agarose gel electrophoresis. The T7P-EGFP template sequence and HeLa genomic RNA were used as the control group.

The results show that through the above T7 RNA polymerase transcription system in vitro, the transcription of the target gene has been successfully achieved.

Firstly, the EGFP gene sequence containing the T7 promoter was obtained by PCR amplification, and then the T7P-EGFP gene sequence was used as a template to perform in vitro reverse transcription by T7 RNA polymerase. The HeLa cell genome was used as a control template, and after the transcription was completed, it was detected by agarose gel electrophoresis. Based on the above assays, it has been demonstrated that using T7P-EGFP as a template the in vitro transcription of EGFP RNA sequence.

7.2 Optimization of the Labeling Conditions and Labeling Ability Detection of T7 RNA Polymerase-Promoted Specific RNA Synthesis Using i⁶ATP as the Substrate.

Preliminarily experiments have unveiled that the recognition efficiency of T7 RNA polymerase for i⁶ATP is significantly higher than that of K4. Next, i⁶ATP will be used as a substrate to optimize the labeling conditions when i⁶ATP is incorporated on the target RNA using the T7 RNA polymerase transcription system, and further test the labeling efficiency.

Step 1. Detection of Fidelity of Chimeric T7P-EGFP Towards i⁶ATP Substrate.

EGFP-RNA was transcript with T7 transcription kit (Takara) in vitro. The system was kept at 37° C. for 2 hours. 1-4 extended to 6 hours. Then the templets were digested with DNase 1, 37° C. for 30 minutes. After that, the RNA was precipitated with 75% alcohol at −20° C. overnight. Then the RNA was washed with 75% alcohol and re-dissolved with RNase free DEPC water (20 μl) and detected by 1% agarose gel electrophoresis. RNA concentration was determined with a Nanodrop One microvolume UV-Vis. spectrophotometer.

TABLE 4
Protocol of transcription system in vitro.

| System | Volume |
|---|---|
| T7P-EGFP gene | 1 μg |
| T7 RNA polymease | 1 μl |
| Transcription buffer in vitro | 2 μl |
| Lane 2 (rNTP mixture, 100 mM) | 2 μl |
| Lane 3 (A\C\G\UTP, 100 mM) | 0.5 μl × 4 |
| Line 4 (i⁶ ATP, 100 mM) | 0.5 μl × 1 |
| Line 5 (i⁶ ATP, 100 mM) | 0.5 μl × 2 |
| Line 6 (i⁶ ATP, 100 mM) | 0.5 μl × 3 |
| Line 7 (i⁶ ATP, 100 mM) | 0.5 μl × 4 |
| DEPC water | total 20 μl |

As depicted, it is demonstrated that T7 RNA polymerase could recognize i⁶ATP, although the recognition efficiency is low comparing with rATP.

The construction systems are shown below:

TABLE 5
Protocol for transcription.

| System | Volume |
|---|---|
| T7P-EGFP gene | 1.0 μg |
| T7 RNA polymerase | 1.0 μl |
| Transcription buffer in vitro | 2.0 μl |
| NTP (100 mM) | 2.0 μl |
| i⁶ ATP (100 mM) | 4.0 μl |
| DEPC water | total 20.0 μl |

Then the templets were digested with DNase I, 37° C. for 30 minutes. Subsequently, the RNA was precipitated with 75% alcohol at −20° C. overnight. Then the RNA was washed with 75% alcohol and re-dissolved with DEPC water (20 μl). The RNA will be directly used for the next step.

Step 2. Optimization of the Transcription Efficiency with i⁶ATP.

In order to improve the transcription efficiency of T7P-EGFP under the condition of i⁶ATP addition, the above conditions were optimized.

The results showed that the transcription efficiency of T7P-EGFP is low in the presence of low concentration i⁶ATP in experimental group 1, which is consistent with the previous results. When the same concentration of adenosine triphosphate (ATP) is added, the transcription efficiency of T7P-EGFP is significantly improved, and the transcription efficiency of T7P-EGFP decreases with the increase of the concentration of i⁶ATP, but it is not obvious.

7.3 Detection of the Labeling Efficiency Using Optimized Condition with i⁶ATP Targeting mRNA of T7P-EGFP.

Through the above experiments, it has been demonstrated that i⁶ATP can be recognized by T7 RNA polymerase, and subsequently the in vitro transcription efficiency of EGFP mRNA in the presence of i⁶ATP has been optimized. Next, the efficiency of i⁶ATP transcription into EGFP needs to be further verified, and the prenyl group on i⁶A (4) is specifically labeled by the well-designed fluorescent probe PTAD-DBCO-FITC (8).

The experimental results showed that the control group could not detect the fluorescence signal of PTAD-DBCO-FITC (8), and with the increase of i⁶ATP concentration, the fluorescence intensity of RNA band gradually increased, which indicated that the content of i⁶A (4) in RNA gradually increased. The labeling efficiency of RNA gradually increases.

### 7.4 Construction of T7-RNA Polymerase Eukaryotic Expression System.

In order to realize the detection of RNA labeling efficiency by use of i⁶A (4) in eukaryotic cells, the eukaryotic expression system of T7 RNA polymerase was first constructed. By detecting the insertion level of i⁶A (4) in the downstream target gene mRNA guided by the T7 promoter, the target RNA can be labeled by the addition of i⁶A (4) in eukaryotic cells. If T7 RNA polymerase in eukaryotic cells can recognize i⁶A (4), the change in the transcription profile of the cell at a specific time point can be detected by controlling the detection time when i⁶A (4) is added.

At first stage, detection of the recognition of i⁶A (4) by T7 RNA polymerase in the eukaryotic cells and the RNA labeling efficiency.

### Step 1. Construction Map of T7-RNA Polymerase Eukaryotic Expression System.

In order to realize the simultaneous expression of T7 RNA polymerase and its target gene in the same cells, the T7 RNA polymerase and EGFP gene expression system were constructed into the same eukaryotic expression system. As shown below, T7 RNA polymerase is transcribed and expressed by the strong eukaryotic promoter CMV, and the EGFP gene sequence is guided by the T7 promoter. In addition, because T7 RNA polymerase transcription produces mRNA lacking the ribosome recognition sequence in eukaryotic cells, the translation process cannot be realized in eukaryotic cells. Therefore, the 5′-end of the EGFP gene is connected to an IRES2 sequence, and the IRES2 transcription sequence can guide the ribosome to downstream genes. Translation process to realize the expression process of EGFP in eukaryotic cells such as tumor cells. In addition, in order to detect the expression of EGFP and T7 RNA polymerase and the relationship between the two, respectively, an expression system for EGFP and T7 RNA polymerase was constructed.

### Step 2. Detection of Expression of T7 RNA Polymerase and EGFP in HeLa Cells.

Next, the T7 RNA polymerase constructed above and the expression level of the EGFP eukaryotic expression system mediated by T7 RNA polymerase in tumor cells were further tested. To this end, the above system is transfected into HeLa cells through a cation-mediated transfection reagent. After 36 hours of transfection, the expression of green fluorescent protein was first detected by fluorescence microscope. Then the cells were lysed by RIPA, the total cell protein was extracted, and the expression of T7 RNA polymerase and EGFP were detected by Western-blotting.

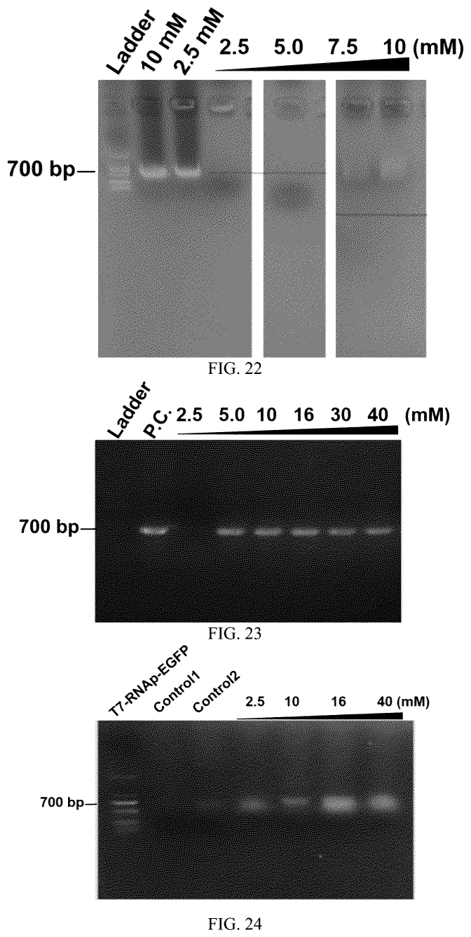

The results showed that T7 RNA polymerase successfully achieved expression in Hela cells. Moreover, the expression of EGFP is T7 RNA polymerase dependent (FIG. 22A), and EGFP expression can only be detected in cells that also express 77 RNA polymerase (FIG. 22B). Therefore, the above experiments show that the EGFP eukaryotic expression system mediated by T7 RNA polymerase has been successfully constructed.

### 7.5 Detection of T7 RNA Polymerase Transcription in Eukaryotic Cells by Fluorescent Expression of EGFP.

In the above experiment, the protein expression of T7 RNA polymerase and EGFP has been detected by Western-blotting. Next, the expression of EGFP was further detected by fluorescence microscope. As in the previous step, the (MV-T7 RNA polymerase and T7 RNA polymerase-IRES2-EGFP transcription systems were transfected into HeLa cells, and the two systems were transfected together. In addition, the (MV-T7 RNA polymerase and T7 RNA polymerase-IRES2-EGFP integration systems were separately transfected into an experimental group. 36 hours after transfection, the expression of EGFP fluorescent protein was detected by fluorescence microscope. The experimental results are consistent with the western-blot detection results, that is, the expression of EGFP is T7 polymerase-dependent. No matter in the (MV-T7 RNA polymerase and T7 RNA polymerase-IRES2-EGFP single transfection group or the two integrations group, only two systems existed concurrently, the expression of green fluorescent protein can only be detected.

The above results demonstrated that the system constructed herein has successfully achieved the T7 RNA polymerase specifically directing the transcription of target genes in eukaryotic cells.

7.6 Detection of i⁶A-Incorporated RNA Labeling Ability in Eukaryotic Cells.

Through the above experiments, we have successfully constructed a T7 RNA polymerase recognition system for i⁶A (4), and realized that i⁶A (4) participates in the transcription of the target gene. In addition, a T7 RNA polymerase-guided eukaryotic transcription system was constructed to realize the eukaryotic transcription process of target genes guided by T7 RNA polymerase. Next, we will further test whether i⁶A (4) can be recognized and participate in the transcription process in eukaryotic cells.

### Step 1. Cytotoxic Assays of i⁶A (4) Towards HeLa Cells.

In order to better detect the involvement of i⁶A (4) in the transcription process of cellular RNA, we also tested the effect of i⁶A (4) on cytotoxicity and optimized the concentration of i⁶A (4) to treat cells.

The experimental results show that the i⁶A (4) concentration within 400 μM has little effect on cell viability. When the concentration reaches 800 μM, cell viability will be significantly inhibited. When the concentration reaches 2.0 mM, i⁶A (4) has a significant inhibitory effect on cells.

### Step 2. Exploration of Whether i⁶A (4) can be Recognized by RNA Polymerase in Eukaryotic Cells.

Next, we will further test whether i⁶A (4) can participate in the RNA transcription process in eukaryotic cells. First culture the HeLa cells in a six-well plate to 70%-80%. Then add different concentrations of i⁶A (4) to DMEM medium (100 μM, 200 μM and 400 μM of i⁶A). After 12 hours of culture, collect all cells and extract total RNA.

RNA (i⁶A-incorporated) labelled by fluorescein PTAD-DBCO-FITC (8).

To the 1.5 mL Eppendorf tube containing PTAD-DBCO-FITC precursor (7, 10 μL, 10 mM solution in DMF) was added N-bromo succinimide (1.0 μL, 100 mM in DMF). The mixture was vortexed gently and formation of the light-red color was observed. The reagent was kept on ice and used for the i⁶A-incorporated RNA labelling immediately. Labeling protocol is the same stated above.

After labeling process, remove excess PTAD-DBCO-FITC (11) dye to determine whether i⁶A (4) can be transcribed into RNA efficiently.

The analysis of the results showed that under the conditions of GoldView™ nucleic acid dye staining, it was detected that the total RNA image in the cell with addition of i⁶A (4) was better than that of the wild type. In addition, its 5s, 18s RNA bands moved up significantly compared with the wild type, while the 28s band did not change significantly.

Through PTAD-DBCO-FITC (8) dye staining, it was found that no fluorescence signal was detected in the control group, but significant fluorescence signal was detected in the total RNA of the cells in the presence of i⁶A (4), and the addition of i⁶A (4, 800 μM) resulted in a weaker RNA fluorescence signal, which may be due to i⁶A (4, 800 μM) has certain cytotoxicity to cells. Through this experiment, we have demonstrated that i⁶A (4) can be recognized in eukaryotic cells and participate in the RNA transcription process.

7.7 Labeling of i⁶A-Incorporated RNA In Vivo.

HeLa cells was obtained from ATCC. HeLa cells were cultured in DMED medium with 10% fetal calf serum. Cells were maintained in a humidified incubator at 5% CO₂ and 37° C. To label RNA in vivo, HeLa cells were cultured in 6-well plates to 70%-80%. Then different concentration i⁶A (4) was added to DMEM medium (i⁶A: 100 μM, 200 μM, 400 μM). After 12 hours, all of the cells were collected and total RNA was extracted with total RNA extraction kit (Takara). The RNA was further labeled with PTAD-DBCO-FITC (8) as described. Then it will be detected by agarose gel electrophoresis.

### 8. Procedure for Labeling of RNA (i⁶A-Incorporated) Using Fluorescent PTAD-DBCO-Cy5 (10)

To the 1.5 mL Eppendorf tube containing PTAD-DBCO-Cy5 precursor (9, 10 μL, 10 mM solution in DMF) was added N-bromo succinimide (1.0 μL, 100 mM in DMF). The mixture was vortexed gently and formation of the light-red color was observed. The reagent was kept on ice and used for the i⁶A-incorporated RNA labelling immediately. The i⁶A-incorporated total RNA (10 μl, 20 ng/μl in DEPC water) was incubated with PTAD-DBCO-Cy5 (10, 10 μl, 0.1 mM), shaken for 30 minutes at 0° C. RNA was analyzed by gel electrophoresis on 1% agarose. The electrophoresis conditions were as follows: power supply (DYY-6C power supply, Liuyi Biotechnology) was set to 130 V. 1% agarose gels were run at room temperature (25° C.) for 20 minutes and stained with or without GoodView™ (SBS Genentech, Beijing, China). A DNA size marker (1 kb DNA Ladder, TsingKe Biotech, TSJ102, Beijing, China) was used. Gels were analyzed by in-gel fluorescence measurements on a FluorChem® FC3 imager (Alpha Innotech). Fluorescence was measured with a blue light excitation wavelength (475 nm) and a green filter emission (537 nm).

### 9. Investigation of I₂-Mediated Cyclisation of i⁶A (4)

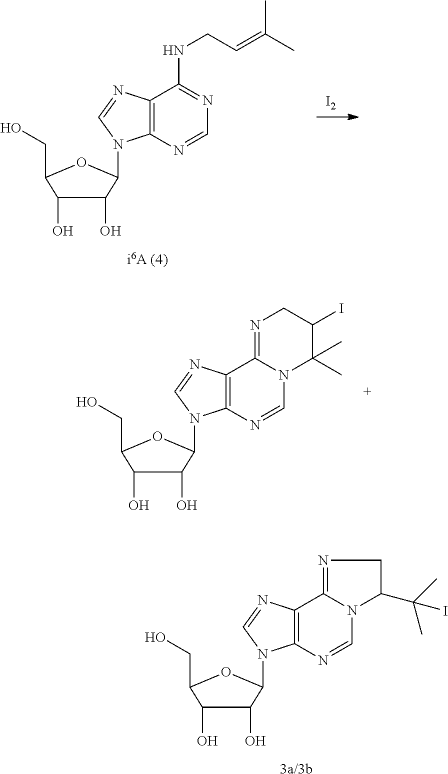 [SMILES: CC(C)=CCNc1ncnc2c1ncn2C1OC(CO)C(O)C1O ; "I2" ; "i6A (4)" ; CC1(C)C(I)CN=C2c3ncn(C4OC(CO)C(O)C4O)c3N=CN21 ; CC(C)(I)C1CN=C2c3ncn(C4OC(CO)C(O)C4O)c3N=CN21 ; "3a/3b"]

Model reactions of i⁶A nucleoside (4). The model reactions were carried out in DMSO-d₆ in order to facilitate in situ NMR characterization without further purification. i⁶A (4, 0.15 mmol) was dissolved in DMSO-d₆ (0.5 mL), and iodine (0.45 mmol) was added. The mixture was stirred at 37° C. for 5 minutes and was taken out for ¹H NMR.

### 9.1 HPLC and Mass Investigations.

The nucleoside i⁶A (4, 0.15 mmol) was dissolved in DMSO (1.0 mL), and iodine (0.45 mmol) was added. The mixture was stirred at 37° C. for 1 hour. Afterwards, saturated Na₂S₂O₃ in H₂O was titrated into the solution in order to remove the excess iodine, and then Na₂CO₃ in H₂O was added. The resultant mixture was further stirred at 37° C. for 1 hour and then subjected to HPLC. Separation was achieved using an Ultimate XB-C₁₈ column, 4.6×150 mm 5 micron (Welch Materials Inc., Shanghai, China) by gradient elution at 0.5 mL/min from 5% to 90% acetonitrile in water (constant 0.1% formic acid) over 15 minutes, isocratic elution with 90% acetonitrile from 15 to 18 minutes, and returned to initial conditions and equilibrated for 5 minutes).

9.4 DFT Calculations of the Iodocyclisation Reaction of i⁶A (4) with Iodine.

Gene sequences used herein.

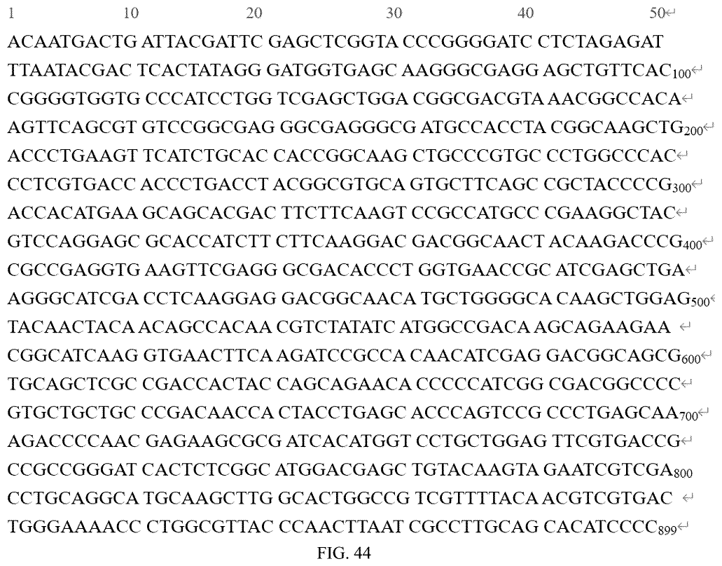

Templated EGPF sequence used here is shown in FIG. 44.

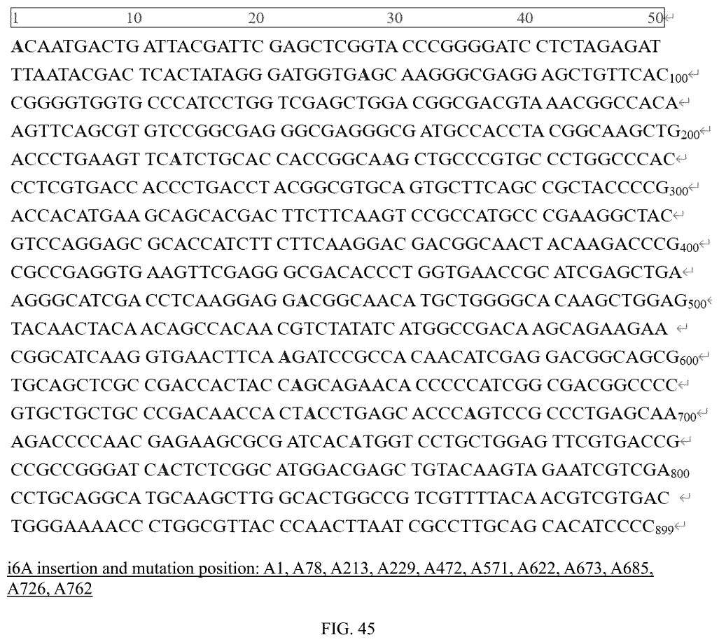

Mutant results for mRNA incorporating i⁶A (templated with EGPF) is shown in FIG. 45. i⁶A insertion and mutation positions: A1, A78, A213, A229, A472, A571, A622, A673, A685, A726, A762.

### Material and Methods

General information. All chemicals were purchased from Sigma-Aldrich, Acros, Inno-chem, Macklin Inc, Energy Chemical and were used without further purification. Extra dry solvents, such as 1,4-dioxane, DMF, and THF, were obtained from Inno-chem in sealed bottles over 3 or 4 Å molecular sieves and stored under dried nitrogen. Organic solvents were obtained from Sinopharm Chemical Reagent Co., Ltd (Shanghai, China) and used in reactions, column chromatography and recrystallizations. Milli-Q ultrapure water (resistivity, 18 m (2) purified through Millipore Milli-Q Advantage A1 purification system was used for all bioconjugation reactions. The reactions were monitored by thin-layer chromatography (TLC) analysis using silica gel (60-Å pore size, F254, Yantai Chemical Industry Research Institute) plates. Compounds were visualized by UV irradiation (λ=254 nm) and/or spraying TLC stain such as a KMnO₄ solution followed by electronic heating. Flash chromatography columns were performed on silica gel (60-Å pore size, 230-400 mesh). HPLC purification were performed using EasyChrom-1000 system with NU3000 serials UV/Vis. detector (Hanbon Sci. & Tech., Jiangsu, China) using an Ultimate® XB-C₁₈ column, 21.2×250 mm 5 micron (Welch Materials Inc., Shanghai, China). Separation was achieved by gradient elution from 5% to 70% acetonitrile in water (constant 0.1% formic acid) over 20 minutes, isocratic elution with 70% acetonitrile from 20 to 25 minutes, and returned to initial conditions and equilibrated for 5 minutes. The LC chromatograms were recorded by monitoring absorption at 254 nm and 220 nm. ¹H and ¹³C NMR spectra were recorded at room temperature on a Bruker spectrometer (AM-600 or AM-400) operating at 600/400 and 150/100 MHz, respectively. Chemical shifts are given in parts per million, and ¹H and ¹³C NMR spectra were referenced using the solvent signal as an internal standard. The following abbreviations are used for the proton spectra multiplicities: s: singlet, d: doublet, t: triplet, m: multiplet, br: broad. Coupling constants (J) are reported in Hertz (Hz). HRMS (TOF) were obtained from the Bruker Micro TOF II Spectrometer using Electro Spray Ionization (ESI). Advion MS (ESI) were obtained from the Expression L (Beijing Bohui Innovation Biotechnology Co., Ltd). LC-MS analysis was obtained from the Bruker Orbitrap LC/MS (Q Exactive™) at Huazhong University of Science and Technology Analytical and Testing Center. UV-visible absorbance measurements were performed with or using UV-visible spectrometer (Lambda365, PerkinElmer, German). Confocal Imaging was performed using a LSM 780 confocal microscope (Zeiss) with a 20× objective at 16-bit depth under non-saturating conditions. EGFP were imaged with a 480 nm (excitation) and a 510 nm (emission) and false-colored green.

Materials. Streptococcus pyogenes (product #M0646), T7 Endonuclease I (product #M0302), Ribonucleotide solution mix (NTPs) and deoxy-ribonucleoside triphosphates (dNTPs) were purchased from New England Biolabs (USA). Transcript Aid T7 High Yield Transcription kit (product #K0441) and Glycogen (product #R0561) were purchased from Thermo Fisher Scientific. Pyrobest™ DNA Polymerase and PrimeSTAR HS DNA Polymerase were purchased from TaKaRa Shuzo Co. Ltd. (Tokyo, Japan). DNA Clean & Concentrator™-5 kit (product #D4014) was purchased from Zymo Research Corp. The DNeasy Blood & Tissue Kit was purchased from QIAGEN. The oligonucleotides at HPLC purity were obtained from TaKaRa company (Dalian, China). The nucleic acid stains Super GelRed (NO.: S-2001) was bought from US Everbright Inc. (Suzhou, China). Thiazolyl Blue Tetrazolium Bromide (MTT, CAS #298-93-1) were purchased from Sigma-Aldrich Inc. (Shanghai, China). DPBS (CAS #63995-75-5) was purchased from TCI (Shanghai) Development Co., Ltd.

Polymerase chain reaction protocol. The concentration of DNA or RNA was quantified by NanoDrop 2000c (Thermo Scientific, USA). Polymerase Chain Reaction was conducted using a A300 Fast Gradient Thermal Cycler (Long Gene Scientific Instruments, Hangzhou, China). Briefly, each reaction was performed under conditions of initial denaturation at 94° C. for 3 minutes, followed by 30 cycles of denaturation (94° C. for 30 seconds), annealing (63° C. for 30 seconds) and extension (72° C. for 60 seconds) and a final extension at 72° C. for 10 minutes.

Electrophoresis protocol. Agarose were purchased from Biofroxx (German). 50× Tris-Acetate EDTA (TAE) buffer (40 mM Tris-acetate and 1 mM EDTA, pH 8.0) were purchased from Biosharp (China). 8 μL of each amplification product was separated by gel electrophoresis on 1% agarose. The electrophoresis conditions were as follows: power supply (DYY-6C power supply, Liuyi Biotechnology) was set to 130 V. 1% agarose gels were run at room temperature (25° C.) for 20 minutes and stained with or without GoodView™ (SBS Genentech, Beijing, China). A DNA size marker (1 kb DNA Ladder, TsingKe Biotech, TSJ102, Beijing, China) was used. Gels were analyzed by in-gel fluorescence measurements on a FluorChem® FC3 imager (Alpha Innotech). Fluorescence was measured with a blue light excitation wavelength (475 nm) and a green filter emission (537 nm) and/or a red-light excitation wavelength (632 nm) and a far-red filter emission (710 nm).

### Functional Probes Synthesis.

The green fluorescent probe PTAD-DBCO-FITC (8).

A solution of compound PTAD-N₃ (10.1 mg, 0.04 mmol) and DBCO-FITC (5.0 mg, 0.01 mmol) in dry N,N-dimethylformamide (DMF, 1.0 mL) under nitrogen was stirred at room temperature overnight. The reaction mixture was purified by RP-HPLC (Separation was achieved using an Ultimate XB-C₁₈ column, 21.2×250 mm 5 micron (Welch Materials Inc., Shanghai, China) by gradient elution from 5% to 70% MeCN in water (constant 0.1% formic acid) over 20 minutes, isocratic elution with 70% MeCN from 20 to 30 minutes, and returned to initial conditions and equilibrated for 5 minutes) to give compound PTAD-DBCO-FITC precursor (6.0 mg, 0.006 mmol, 66%, retention time 22 minutes) as yellow solid. ¹H NMR (400 MHz, methanol-d⁴) δ 8.23 (s, 1H), 7.99 (d, J=8.0 Hz, 1H), 7.54 (d, J=8.0 Hz, 2H), 7.45 (s, 2H), 7.29 (d, J=4.0 Hz, 1H), 7.25-7.23 (m, 3H), 7.29 (t, J=8.0 Hz, 2H), 7.15 (d, J=8.0 Hz, 1H), 6.95 (d, J=8.0 Hz, 1H), 6.86 (d, J=8.0 Hz, 1H), 6.60 (s, 2H), 6.51 (q, J=12.0 Hz, 2H), 6.46-6.42 (m, 2H), 5.94 (d, J=20.0 Hz, 1H), 4.42 (t, J=16.0 Hz, 1H), 4.36 (t, J=8.0 Hz, 1H), 3.40-3.30 (m, 2H), 2.24-2.08 (m, 2H), 1.96-1.74 (m, 2H). HRMS (TOF) Calculated for [M+Na]⁺=919.2452. found=919.2436. The in situ oxidation of the PTAD-DBCO-FITC precursor in N,N-dimethylformamide (DMF) using an equal equivalent of N-bromosuccinimide (NBS) yielded PTAD-DBCO-FITC (8, 10 mM in DMF, stored at −20° C.). This product was utilized directly without further purification.

The red fluorescent probe PTAD-DBCO-Cy5 (10).

A solution of compound PTAD-N₃ (3.4 mg, 0.0128 mmol) and DBCO-Cy5 (5.0 mg, 0.0064 mmol) in dry CH₃CN (1.0 mL) under nitrogen was stirred at room temperature overnight. The reaction mixture was purified by RP-HPLC (Separation was achieved using a Ultimate XB-C₁₈ column, 21.2×250 mm 5 micron (Welch Materials Inc., Shanghai, China) by gradient elution from 5% to 70% MeCN in water (constant 0.1% formic acid) over 15 min, isocratic elution with 70% MeCN from 15 to 30 minutes, and returned to initial conditions and equilibrated for 5 minutes) to give PTAD-DBCO-Cy5 precursor (6.0 mg, 0.0057 mmol, 90%, retention time 24 minutes) as blue solid, which was re-dissolved in 0.3 mL of anhydrous N,N-dimethylformamide (DMF) and subsequently treated with an equal equivalent of N-bromosuccinimide (NBS) solution (20 mM in DMF) to formulate a PTAD-DBCO-Cy5 stock solution (10, 10 mM in DMF, stored at −20° C.). MS (ESI) Calculated for [M−H]⁻=1035.5. found [M−H]⁻=1034.7. HRMS (TOF) Calculated for [M−Cl]⁺=1003.4977. found 1003.4964.

### Nucleotide i⁶A Triphosphate Synthesis.

Nucleoside i⁶A (35 mg, 0.1 mmol) and proton sponge (58 mg, 0.25 mmol) were dissolved in trimethyl phosphate (0.7 mL) and was placed under the ice-water bath. phosphorus oxychloride (33.6 μL, 0.36 mmol) was slowly added and stirred at 0° C. for 12 hours. A solution of tributylamine (175 μL, 0.72 mmol) and tributyl ammonium pyrophosphate (642 mg) in DMF (2.0 mL) was slowly added and the reaction mixture was stirred at 0° C. for 30 minutes. The reaction was quenched by addition of 1.0 M aqueous TEAB (pH=7.5, 15 mL). The mixture was diluted with H₂O (5.0 mL) and subjected to HPLC purification. Separation was achieved using an Ultimate XB-C₁₈ column, 21.2×250 mm 5 micron by gradient elution from 5% to 50% MeCN in water (constant 0.1% formic acid) over 25 minutes, isocratic elution with 50% MeCN from 25 to 30 minutes, and returned to initial conditions and equilibrated for 5 minutes to give i⁶ATP (10.0 mg, as tributyl ammonium salt, retention time 20 minutes). ¹H NMR (400 MHz, methanol-d⁴) δ 8.40 (s, 2H), 8.30 (s, 1H), 6.13 (d, J=4.0 Hz, 1H), 5.40 (t, J=12.0 Hz, 2H), 4.73 (q, J=16.0 Hz, 1H), 4.57-4.43 (m, 1H), 4.27-4.26 (m, 2H), 4.20-4.13 (m, 2H). The proton peak of methyl group in prenylated functionality was covered by tributyl ammonium salt in ¹H NMR. ³¹P NMR (162 MHz, methanol-d⁴) δ −10.47 (d, J=22.68 Hz, α-P), −11.46 (d, J=22.68 Hz, γ-P), −24.00 (t, J=43.74 Hz, β-P). MS (ESI) Calculated for [M−H]⁻=574.1. found [M−H]⁻=574.2.

### Profiling the Dynamic of the Ene-Ligation Between i⁶A Nucleoside and PTAD.

The nucleoside i⁶A (10 mg, 0.03 mmol) was dissolved in a mixture of CD₃CN and D₂O (0.5 mL, volume-to-volume ratio of 4:1, resulting in a final concentration of 60 mM). Subsequently, 4-phenyl-3H-1,2,4-triazole-3,5(4H)-dione (PTAD, 26 mg, 0.15 mmol, equivalent to 5 times the amount of i⁶A) was added to the solution (resulting in a final concentration of 300 mM). The reaction mixture was then allowed to stand at room temperature. The ¹H NMR spectra were obtained using a 400 MHz ¹H NMR instrument with CD₃CN/D₂O as the solvent.

### Fluorescence Labeling of i⁶A in the Metabolites of Eukaryotic Cells.

The HeLa cells are first cultured in a six-well plate until they reach a confluence of 70%-80%. Different concentrations of i⁶A (4) are then added to the DMEM medium, consisting of 100 μM, 200 μM, and 400 μM of i⁶A. After a 12-hour incubation period, all cells are collected, and total RNA is extracted from them.

RNA incorporating i⁶A was labeled using fluorescein PTAD-DBCO-FITC (8).

To In a 1.5 mL Eppendorf tube containing the PTAD-DBCO-FITC precursor (7, 10 mL of a 10 mM solution in DMF), N-bromosuccinimide (1.0 mL of a 100 mM solution in DMF) was added. The mixture was gently vortexed, and the formation of a light-red color was observed. The reagent was kept on ice and immediately used for labeling the i⁶A-incorporated RNA.

The i⁶A-incorporated total RNA (10 mL, 20 ng/mL in DEPC water) was incubated with PTAD-DBCO-FITC (8, 10 mL of a 0.1 mM solution), shaken for 30 minutes at 0° C. The RNA was then analyzed through gel electrophoresis on a 1% agarose gel. The electrophoresis conditions were as follows: power supply (DYY-6C power supply, Liuyi Biotechnology) was set to 130 V. 1% agarose gels were run at room temperature (25° C.) for 20 minutes and stained with or without GoodView™ (SBS Genentech, Beijing, China). A DNA size marker (1 kb DNA Ladder, TsingKe Biotech, TSJ102, Beijing, China) was used. Gels were analyzed by in-gel fluorescence measurements on a FluorChem® FC3 imager (Alpha Innotech). Fluorescence was measured with a blue light excitation wavelength (475 nm) and a green filter emission (537 nm).

RNA containing i⁶A was labeled using fluorescein PTAD-DBCO-Cy5 (10).

In a 1.5 mL Eppendorf tube, the PTAD-DBCO-Cy5 precursor (9, 10 mL of a 10 mM solution in DMF) was mixed with N-bromosuccinimide (1.0 mL of a 100 mM solution in DMF). The mixture was gently vortexed, and the formation of a light-red color was observed, indicating successful reaction. The prepared reagent was then kept on ice and used immediately for labeling the i⁶A-incorporated RNA. The i⁶A-containing total RNA (10 mL, 20 ng/mL in DEPC water) was incubated with the activated PTAD-DBCO-Cy5 (10, 10 mL of a 0.1 mM solution), and the mixture was shaken for 30 minutes at 0° C. to facilitate labeling. Following this incubation, the labeled RNA was analyzed through gel electrophoresis on a 1% agarose gel. The electrophoresis conditions were as follows: power supply (DYY-6C power supply, Liuyi Biotechnology) was set to 130 V. 1% agarose gels were run at room temperature (25° C.) for 20 minutes and stained with or without GoodView™ (SBS Genentech, Beijing, China). A DNA size marker (1 kb DNA Ladder, TsingKe Biotech, TSJ102, Beijing, China) was used. Gels were analyzed by in-gel fluorescence measurements on a FluorChem® FC3 imager (Alpha Innotech). Fluorescence was measured with a blue light excitation wavelength (475 nm) and a green filter emission (537 nm).

### The Cyclization of Nucleoside i⁶A Mediated by Iodine.

The reactions were conducted in DMSO-d⁶ to enable in situ NMR characterization without the need for additional purification. i⁶A (4, 0.15 mmol) was dissolved in DMSO-d⁶ (0.5 mL), and iodine (0.45 mmol in DMSO-d⁶) was subsequently added to the solution. The mixture was then stirred at 37° C. for 5 minutes. After this incubation period, the reaction mixture was subjected to ¹H NMR, ¹³C NMR, HPLC, UV and LC-MS analysis.

### Prediction of the Plausible Reaction Pathway for Iodine-Mediated Cyclization of Nucleoside i⁶A.

All calculations were conducted using the Gaussian 16, Revision B.01 software program. The geometry optimization of the model systems in the gas phase was performed utilizing the B3LYP/BSI Density Functional Theory (DFT) method, which was augmented with the D3 (BJ) version of Grimme's empirical dispersion correction. In this context, BS1 refers to a basis set that combines the SDD for iodine atoms and the 6-31G (d, p) for all other atoms. Frequency calculations were carried out at the same theoretical level to ascertain whether the optimized structures represent minima (absence of imaginary frequencies) or saddle points (presence of a single imaginary frequency) on the potential energy surface. These calculations also provided thermal corrections to the Gibbs free energies. The solvation energy corrections were computed at the B3LYP/BS1 level employing the SMD solvation model for DMSO, based on the gas-phase optimized geometries. Single point energies were calculated using the M062X/BS2 method, which was also augmented with the D3 version of Grimme's empirical dispersion correction. In this case, BS2 denotes a basis set combining the SDD for iodine atoms and the Def2TZVP for all other atoms.

Illustrations of the profiling methods for i⁶A residues in cells.

In Vitro Detection of the Efficiency of i⁶ATP in the T7 RNA Polymerase Transcription System.

The EGFP mRNA sequence was obtained by transcribing under the conditions of i⁶ATP: ATP ratio of 0:1, 3:7, 7:3, 1:0 through the in vitro transcription system, and then detected each group of EGFP RNA sequence through iodine (0.5 M) treatment. After processing, the cDNA sequence of EGFP in each group was obtained by reverse transcription. Next, the obtained cDNA sequence was used as a template, and the mutant EGFP gene sequence in each group was obtained by PCR amplification, and cloned into the T vector. Through the amplification of DH5α E. coli., 50 clones were picked from each group and sequenced to detect the mutation. After the mutation site of EGFP gene, and analyze the insertion efficiency of i⁶ATP.

Comparative Analysis of the Recognition Efficiency of T7 RNA Polymerase and RNA Polymerase II for i⁶ATP in Eukaryotic Cells.

T7 RNA polymerase and T7-IRES2-EGFP expression system were expressed in Hela cells, and RNA was extracted 48 hours after transfection, and then the mixture was treated with iodine to cause i⁶ATP insertion site mutation. Then, the mutant sequences of the two endogenously expressed genes of GAPDH and Notch1 were obtained by reverse transcription, and the mutant gene sequence of EGFP catalyzed by T7 RNA polymerase transcription was obtained. Same as the previous detection process, by detecting the insertion efficiency of i⁶ATP in the endogenous highly expressed gene GAPDH and the oncogene Notch1 and the insertion efficiency of i⁶ATP in T7-EGFP. Compare the recognition efficiency of i⁶ATP by eukaryotic RNA polymerase and the feasibility of using it as a real-time transcriptome marker.

Yeast Cell Culture and tRNA Isolation.

The wild-type and MOD5 deletion strains of Saccharomyces cerevisiae were kindly provided by Dr. Jia-Xing Yue from Sun Yat-sen University Cancer Center. The S. cerevisiae cells were cultured in yeast extract-peptone-dextrose (YPD) medium at 30° C. until reaching the logarithmic growth phase. For H₂O₂ treatment, the cells were exposed to 12 mM H₂O₂ for 1 hour before harvesting, as reported previously (38).

Cells were pelleted by centrifugation, and grinded into fine powders using a mortar and pestle, with the addition of liquid nitrogen. Total RNA was extracted using Trizol (Invitrogen, RN0401) following the manufacturer's instructions. RNAs shorter than 200 nucleotides, primarily consisting of tRNAs, were isolated using the Small RNA clean kit (ZYMO research).

### Measurement of i⁶A Levels by Mass Spectrometry.

To determine the levels of i⁶A in tRNAs, liquid chromatography-tandem mass spectrometry (LC-MS/MS) was performed by Wuhan BioBank in Wuhan, China. Synthesized i⁶A was used as a standard for this analysis.

### IMCR-tRNA-Seq Library Construction.

Initially, 10 μg of tRNAs were treated with 0.1 M Tris-HCl (pH 9.0) at 37° C. for 45 minutes to deacylate the tRNAs. Half of the deacylated tRNAs were treated with 0.5 M I₂ for 15 minutes on ice. Following this, saturated Na₂S₂O₃ was added until the solution became colorless, and saturated Na₂CO₃ was added until it ceased to produce bubbles. Once the reaction was complete, the tRNAs were purified using the RNA Clean & Concentrator Kit (ZYMO Research). Subsequently, both iodine-treated and untreated tRNAs were used for sequencing library construction, following a previously established protocol with some modifications (39). Notably, Induro™ Reverse Transcriptase (NEB, #M0681L) was used instead of TGIRT. The process began with the incubation of deacylated tRNAs with T4 PNK to dephosphorylate their 3′ ends. tRNAs from both samples were then purified using PAGE, followed by the ligation of a 3′ adaptor to the purified tRNAs using T4 RNA Ligase 2, truncated KQ (NEB, M0373L), at 25° C. for 3 hours. After another round of PAGE purification, the ligated products were reverse transcribed using Induro® Reverse Transcriptase (NEB, M0681L) at 55° C. for 16 hours in 1×RT buffer (10 mM MgCl₂, 10 mM DTT, and 75 mM KCl). The resulting cDNA was then purified and circularized using Circligase™-II (Lucigen, #CL9025K). Finally, the libraries were amplified using KAPA HiFi Polymerase (Roche, #KK2602) and sequenced on an Illumina Novaseq 6000 platform. The primer sequences are listed in Table S5.

tRNA-Seq Data Analysis.

Adaptors and random sequences were trimmed, and low-quality sequences were discarded using Cutadapt software (40-41). The resulting clean reads were then processed using a previously published pipeline to quantify and analyze tRNA expression and modifications (39). The mature tRNA sequences of S. cerevisiae were obtained from GtRNAdb. The analysis was performed using the following parameters:
--cluster --cluster-id 0.90 --min-cov 2000 --max-mismatches 0.1 --remap --remap-mismatches 0.075 --control-condition control

To ensure the quality of our tRNA-seq data, we thoroughly examined several key parameters. These included mapping rate, correlation of samples, coverage of tRNAs, and detection of known tRNA modifications.

To identify the i⁶A site in tRNAs, the mutation rate with and without I₂ treatment was calculated to determine the i⁶A score. The i⁶A score is defined as relative possibility of i⁶A modification at a specific site. A larger mutation rate difference corresponds to a higher score. An i⁶A score threshold of 1 was set, with scores greater than 1 indicating the presence of i⁶A modification at the respective site. The score is calculated as follows:

$$Score = \frac{( M t - M c ) * M t}{M c + 0.001}$$

Mt represents the mutation rate with I₂ treatment, and Mc is the mutation rate without I₂ treatment. To ensure efficient calculation of the i⁶A score, Laplace smoothing is incorporated. All customized scripts used in this study are available from the corresponding author upon request.

### Quantification and Statistical Analysis

Two-tailed t-tests or Wilcoxon's signed-rank tests were employed for group comparisons. Correlation analyses were conducted using Pearson tests. A p-value of less than 0.05 was considered statistically significant. Data visualization was accomplished using custom R scripts.

### Results and Discussion

### Prenyl-Transformations and the Development of New Labeling Reagents.

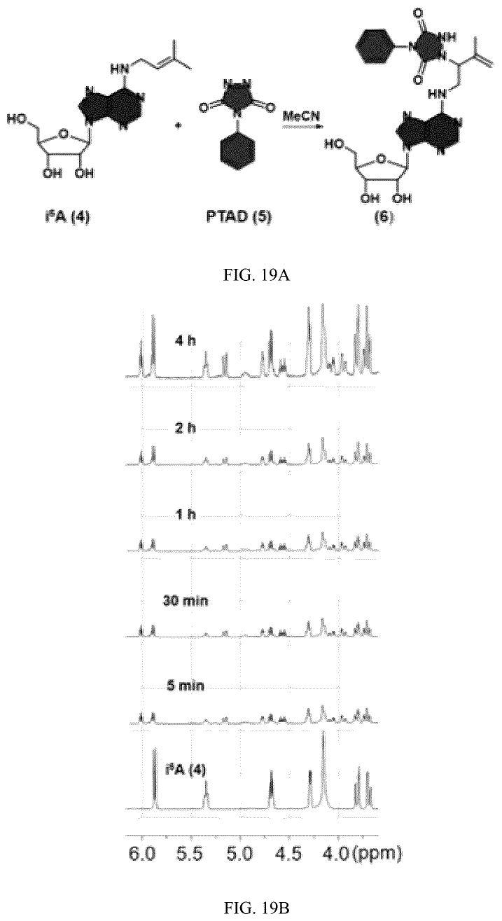

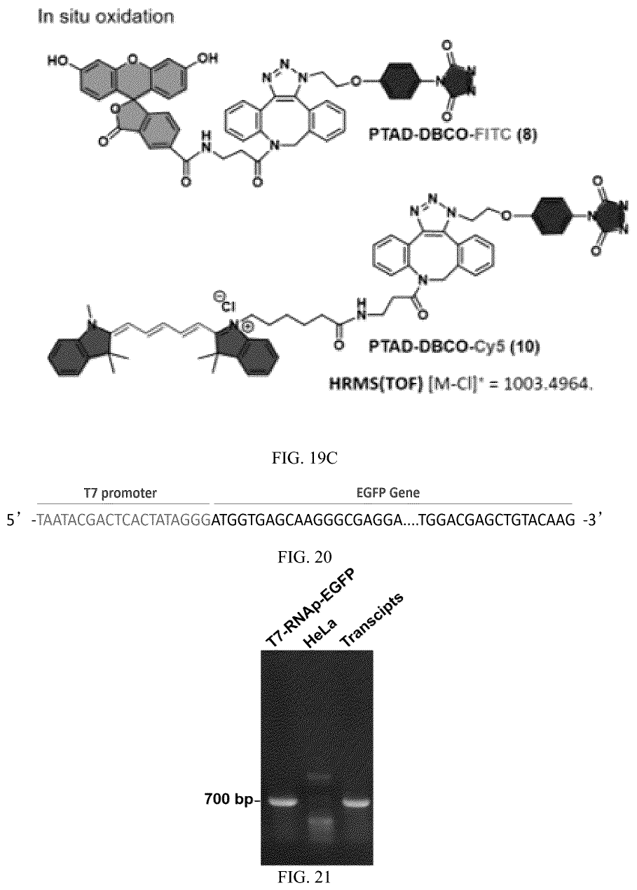

We synthesized the i⁶A nucleoside (4 in FIG. 19) through a linear 4-step process in an overall 69% yield (Scheme S1), and started to test its compatibility with labeling reagents. A rapid reaction was observed between i⁶A (4) and PTAD (5) (FIG. 19A) with a clear color change in less than 5 minutes. The resulting products were monitored by in situ NMR (FIG. 19B), confirming that this reaction can take place quickly with good targeting specificity in biocompatible conditions.

To further expand this method and take advantage of the fluorescence labeling using this chemistry, we constructed two fluorescent probes derived from the structures of PTAD (8 and 10, FIG. 19C, and Scheme S2-3). The strain-promoted click reaction of azido PTAD with FITC-DBCO (Scheme S2) or Cy5-DBCO (Scheme S3) proceeded smoothly to provide fluorescent precursors, followed by the in situ oxidation with N-bromosuccinimide (NBS) to yield fluorescent probes 8 and 10. In the meantime, we synthesized the i⁶A triphosphates (i⁶ATP) for its enzymatic incorporation into RNA strands. Based on previous studies, i⁶ATP was obtained in a linear 2-step process with 30% of isolated yields (Scheme S4). All the data were consistent with the literature results. The peaks in the phosphorus NMR spectra at −10.5, −11.5 and −24.0 ppm were assigned to α, γ and β phosphorus atoms respectively, each with a J value of 26.8 Hz.

### Fluorescence Labeling of i⁶A in the Metabolites of Eukaryotic Cells.

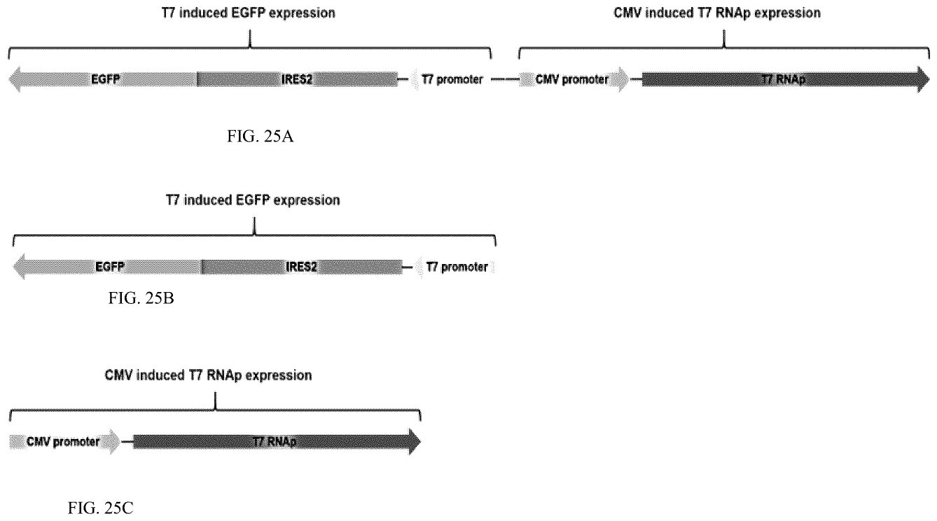

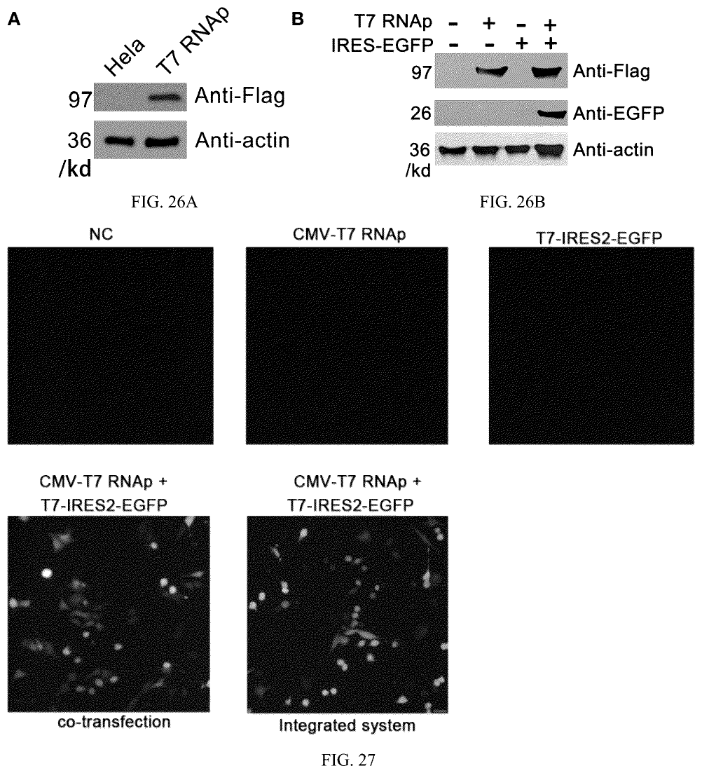

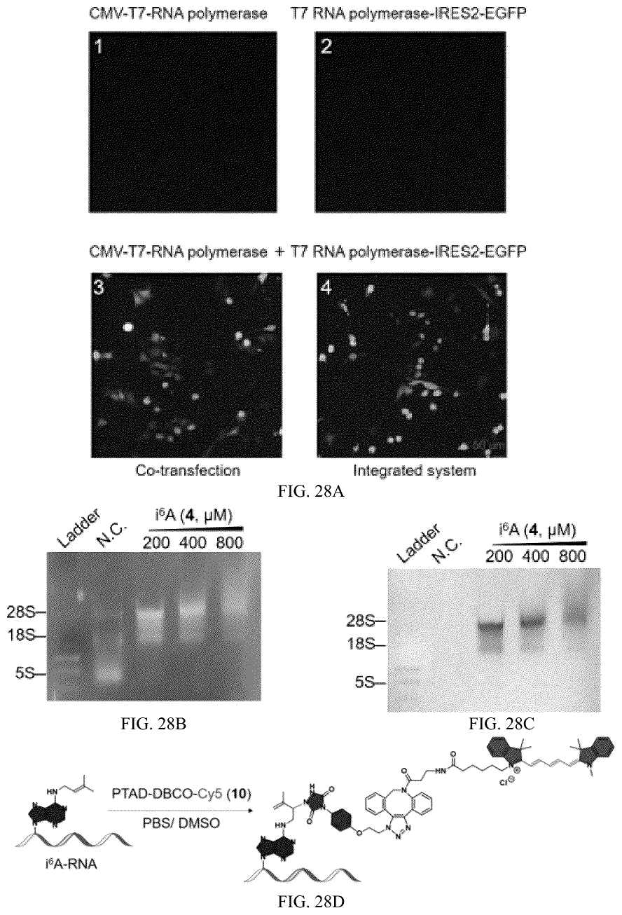

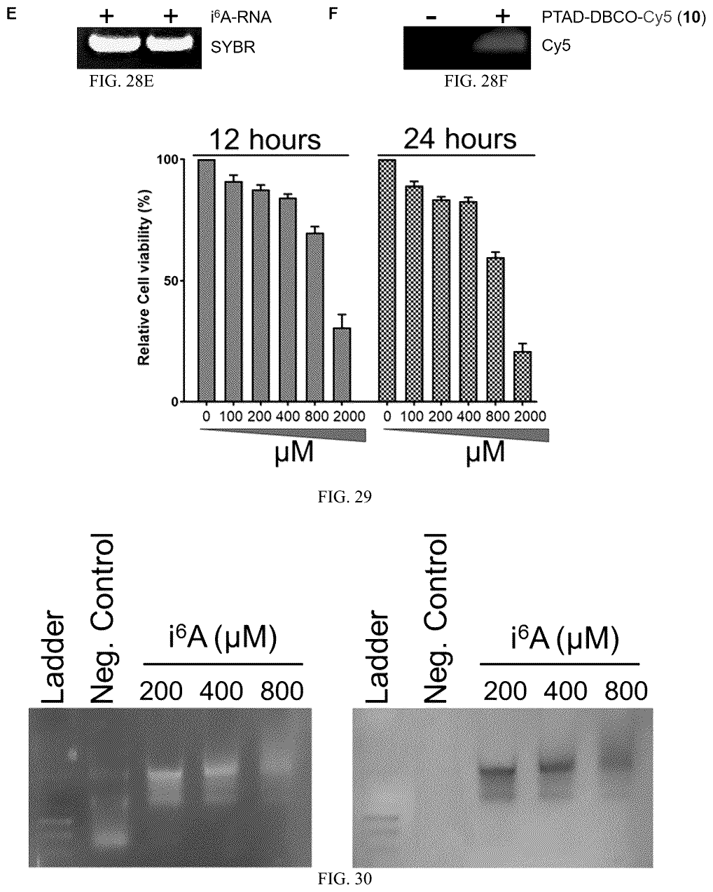

With the two fluorescent labeling reagents and i⁶ATP building block in hands, we asked whether i⁶A nucleoside can be incorporated by RNA polymerases in the process of transcription within eukaryotic cells, and if so can they be recognized and fluorescently labeled (FIG. 28). To investigate this, we cultured HeLa cells to 70%-80% confluence in a 6-well plate, followed by adding different concentrations of i⁶A (4, 100 μM, 200 μM, 400 μM) in DMEM. Subsequently, the CMV-T7 and T7pro-IRES2-EGFP plasmids, as well as their conjugate units (Table S1-3 and FIGS. 20-27) were transfected respectively in HeLa cells for 36 hours before the measurement was taken for the expression of EGFP protein by fluorescence microscopy. As shown in FIG. 28A, the expression of green fluorescent protein can only be detected in the presence of CMV-T7 and T7pro-IRES2-EGFP or their integration counterparts, which indicated the successful formation of specific transcripts of the target genes by T7 RNA polymerase in eukaryotic cells.

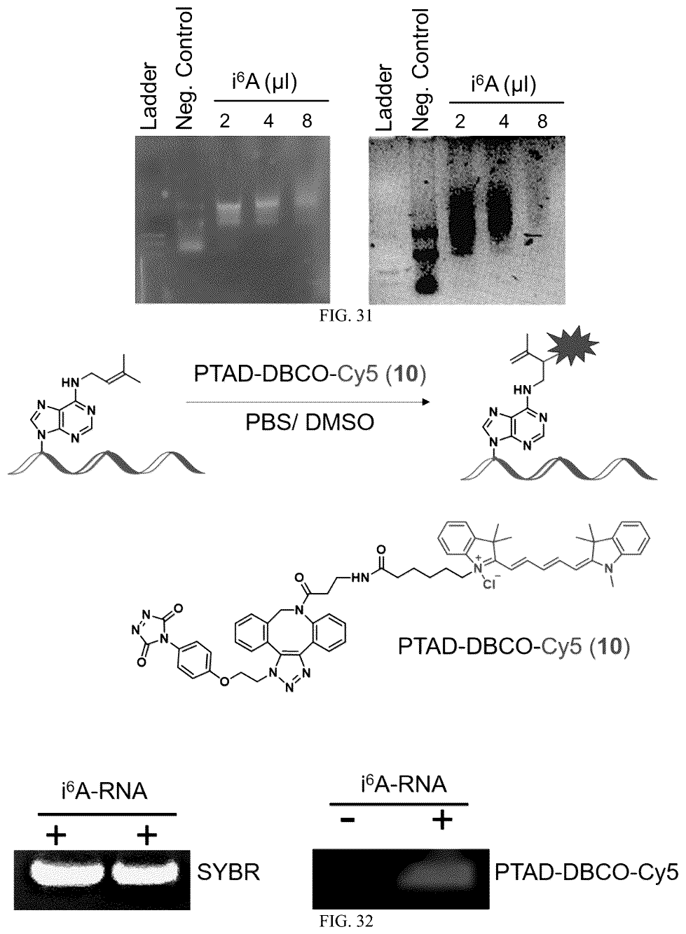

For the labeling experiment, HeLa cells were harvested after the verification of cytotoxic study (FIG. 29) and the total RNAs were extracted 12 hours after the transfection, which were subsequently incubated with our PTAD-DBCO-FITC (8) or PTAD-DBCO-Cy5 (10) respectively. After excess washing, the purified RNAs were evaluated for the incorporation of i⁶A residues, and the overall PTAD-DBCO-FITC (8) fluorescent labeling efficiency was determined by agarose gel electrophoresis (FIGS. 30-32). Both the modified and the wild-type RNAs appeared to have similar stability with i⁶A incorporated as observed in Goldview nucleic acid dye staining (FIGS. 28b-28c). In addition, the rRNA bands representing 5s and 18s were significantly shifted upward compared with the wild-type one. By contrast, no significant change was observed in the rRNA band at 28s. Notably, the fluorescence intensity of the RNAs containing i⁶A decreased when the level of i⁶A reached the cytotoxic concentration of 800 μM (FIGS. 28B-28C and, and 30-32). No fluorescence signal was detected in the control group in comparison to a distinct fluorescence signal observed in the total RNAs incorporated with i⁶A and treated with PTAD-DBCO-Cy5 (13) probe (FIGS. 28D-28F). Notably, the direct in-gel fluorescent labeling of i⁶A-containing transcripts have also been intensively determined (FIG. 28E). Our assays indicated that i⁶A nucleosides could be recognized and used in transcription by RNA polymerase and the resulting RNAs are detectable by our fluorescent probes. Having achieved successful fluorescent labeling, we proceeded to investigate whether chemically inducible sequencing could be accomplished by leveraging the distinct structure of the prenyl functionality.

### Development of Iodine-Mediated Cyclisation and Reverse Transcription (IMCRT) Method for i⁶A Profiling.

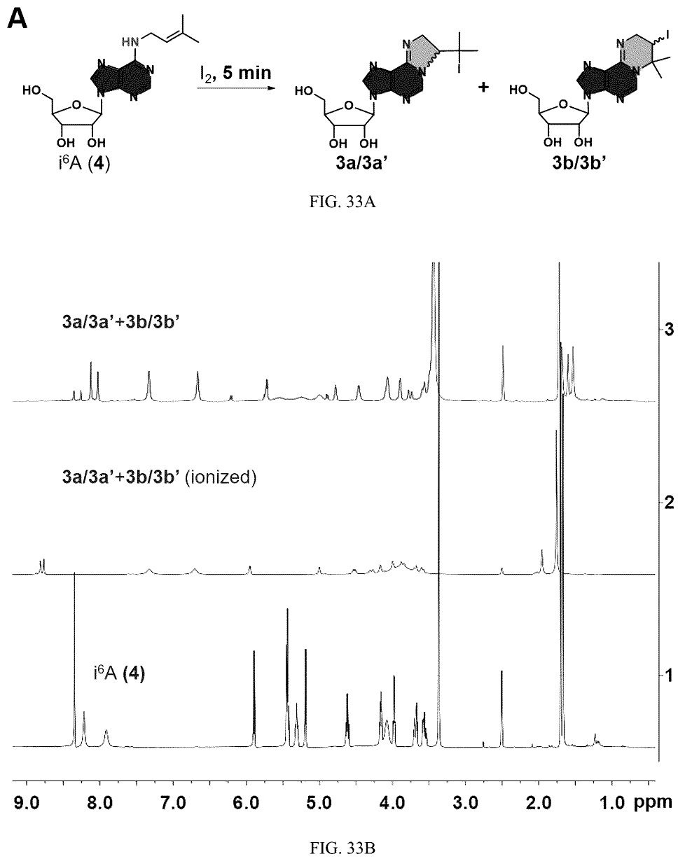

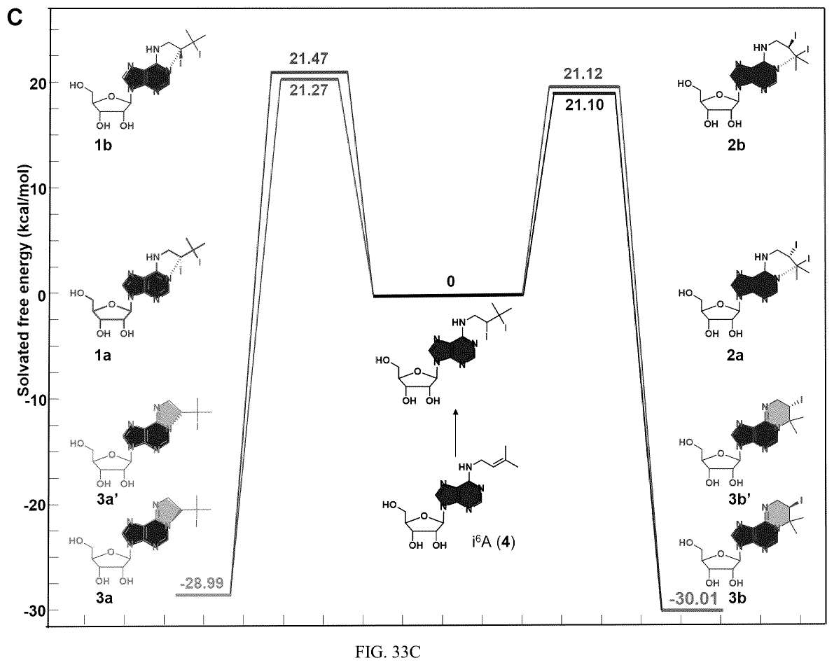

We further examined the prenyl modification on RNAs (35-37) via the addition of I₂, a reagent for the cyclisation reaction of a prenyl group (42-44). As expected, the reaction of i⁶A with optimized concentration of iodine reagent proceeded very smoothly to yield the full conversion of i⁶A in less than 5 minutes (FIGS. 33A-33B). The NMR, mass spectrometry and UV results confirmed the spontaneous formation of the cyclisation adducts 3a or 3b.

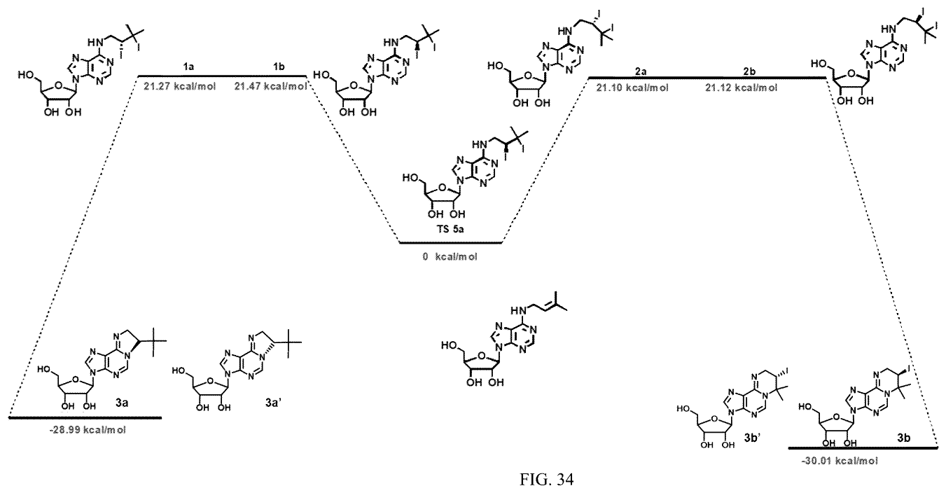

DFT calculations were performed to elucidate the possible reaction pathway and mechanism. The addition of I₂ resulted in the spontaneous attachment of di-iodinyl atoms to the double bond of the prenyl group with four potential isomers formed because of the chirality of the products (1a-1b, 2a-2b) (FIGS. 33C and 34 for details). Two plausible reaction pathways have been identified thus far involving the formation of either five-membered or six-membered intermediates through a nucleophilic displacement process. Between the two products, compound 3b/3b′ has been demonstrated to be more abundant based on the extensive NMR studies (37), even though FIG. 33C showed very small difference among the activation barriers of the four isomers. As a result, the iodine treatment followed by quenching with sodium persulfate converted i⁶A from an H-bond donor to a cyclic H-bond acceptor, which might change the enzyme recognition and cause base mutation during reverse transcription (RT) process, suggesting the potential application for the detection of the prenyl functionality in endogenous RNAs at a single-base resolution.

Profiling of artificially incorporated i⁶A residues in cell.

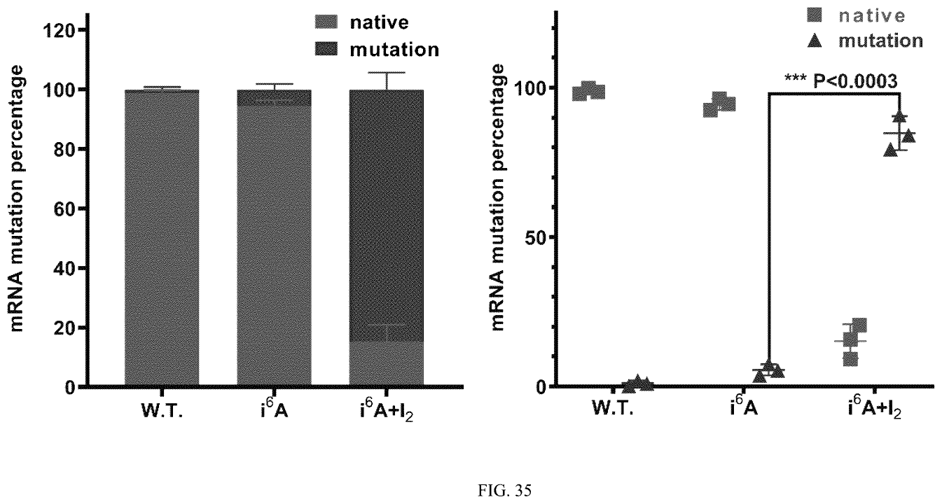

We subsequently applied this IMCRT method to detect i⁶A modification sites in artificial RNAs. The T7P-EGFP gene was utilized as a template for in vitro transcription, employing T7 RNA Polymerase. In the course of cellular metabolism, living HeLa cells were fed with 30% and 70% i⁶A nucleoside in two groups respectively following the optimized protocol. The resulting EGFP RNA from each experimental group was further treated with I₂ (0.5 M). Each EGFP RNA was then reverse transcribed into cDNA followed by PCR amplification. Each amplified EGFP amplicon was next cloned into the T-vector and transformed into DH5α E. coli. A total of 50 clones were picked from each group, followed by high-throughput sequencing and comparing with the native control to determine the sites of mutation on EGFP gene and to analyze the insertion efficiency of i⁶A (FIG. 35). The incorporations of i⁶A into EGFP-mRNA in Hela cells is randomly via salvage pathway using nucleotides i⁶ATP. It was found that i⁶A were settled from head to the middle of the sequences of this EGFP-mRNA (blue color in Table S6 in SI). Deep analysis of i⁶A insertions at specific RNA sites and chemical-induced mutation with I₂ treatment showed that we could distinguish the i⁶A residues from native adenosine sites at single base resolution. As a result, adenosine with prenyl modification displays normal base pairing with thymidine. By contrast, the addition of iodine induced the transformation of i⁶A to its cyclic form, thereby diminishing the normal base pairing specificity and resulting in a mixture of complementary G/C/U (FIG. 36A). The base-mutation rates could be further measured by the sequencing data. It turns out that in the presence of 30% of i⁶ATP, the resulting A bases were significantly changed (FIGS. 36B1 and 36B2), whereas G/C/U displayed statistically minimal effect (FIGS. 36B2 and 36B4). By contrast, the I₂-treated i⁶A shows a significant mutation rate, as shown in FIGS. 36C1 and 36C2, which demonstrated the feasibility of our method to detect the i⁶A sites in RNAs. Similar to the contribution of the bisulfite sequencing method to the biological studies of DNA/RNA methylation, this unique IMCRT sequencing strategy could open a new door for the study of prenylated RNA in terms of sequencing, biological function illustration and beyond.

Characterization of i⁶A Modification in Yeast tRNAs.

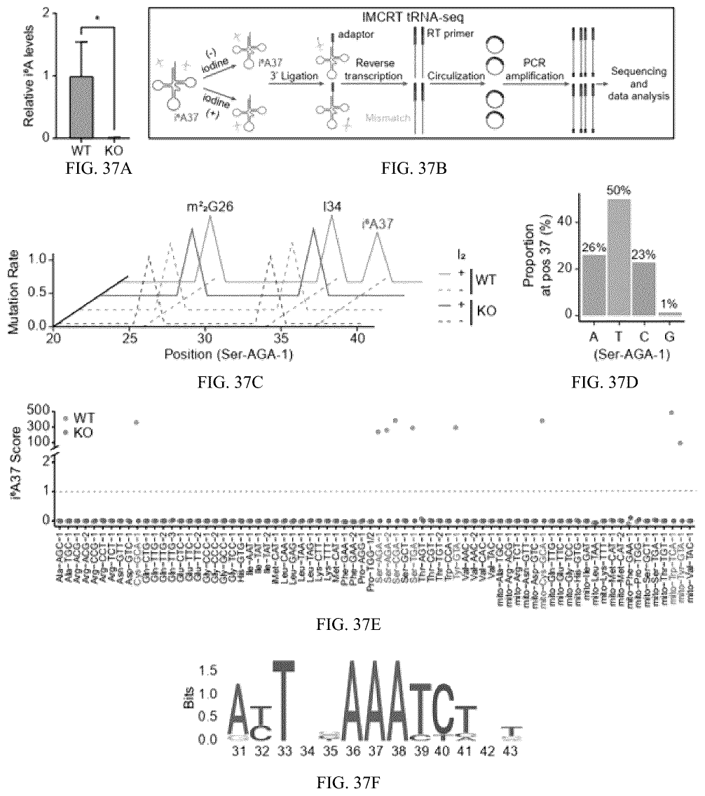

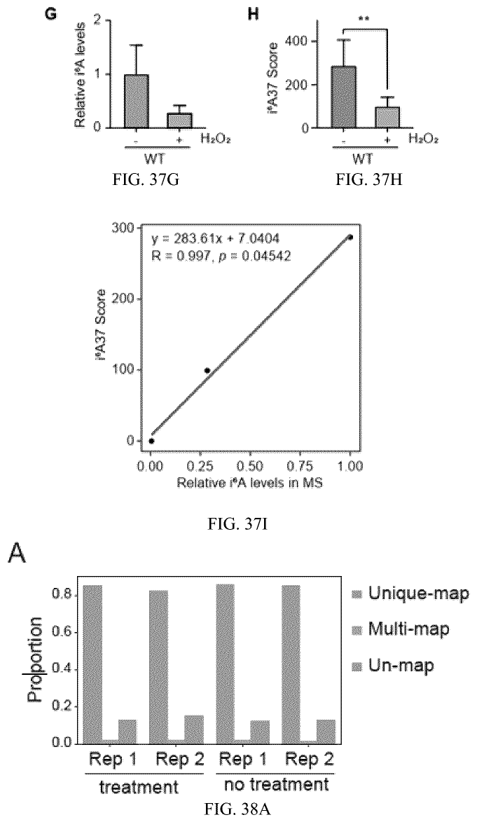

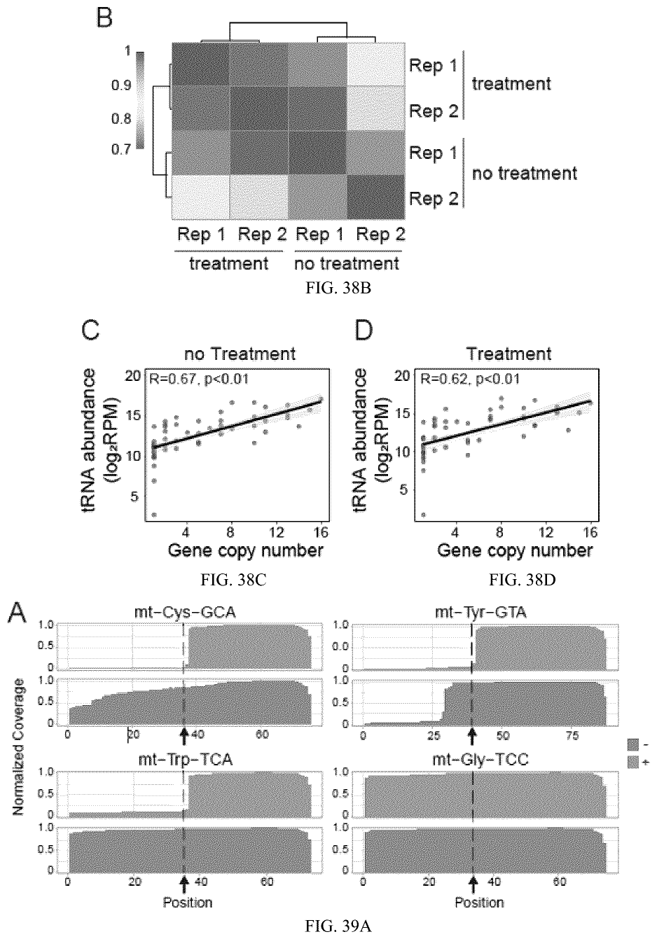

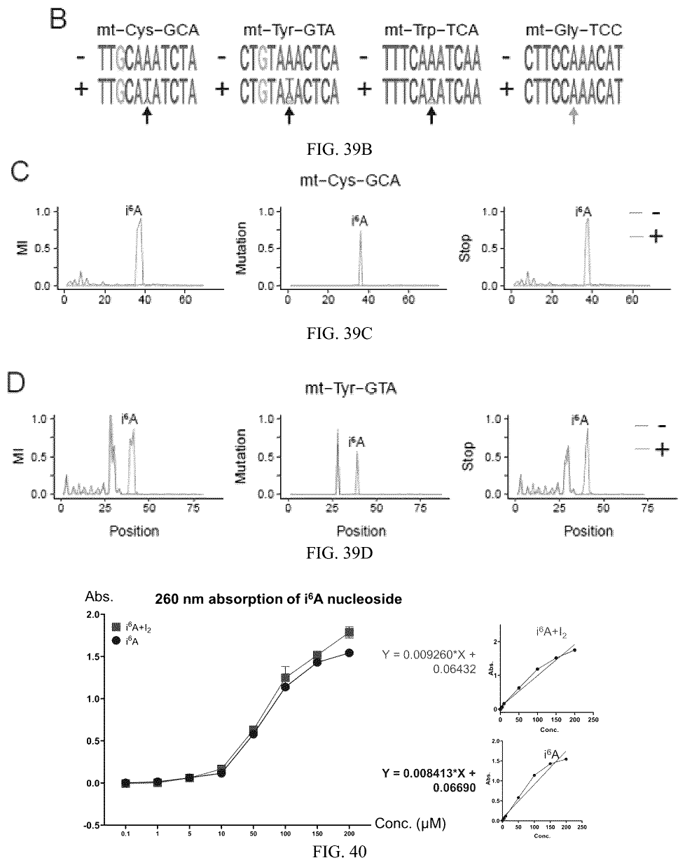

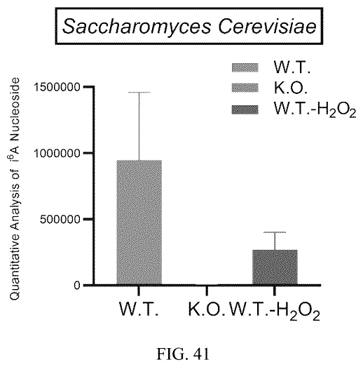

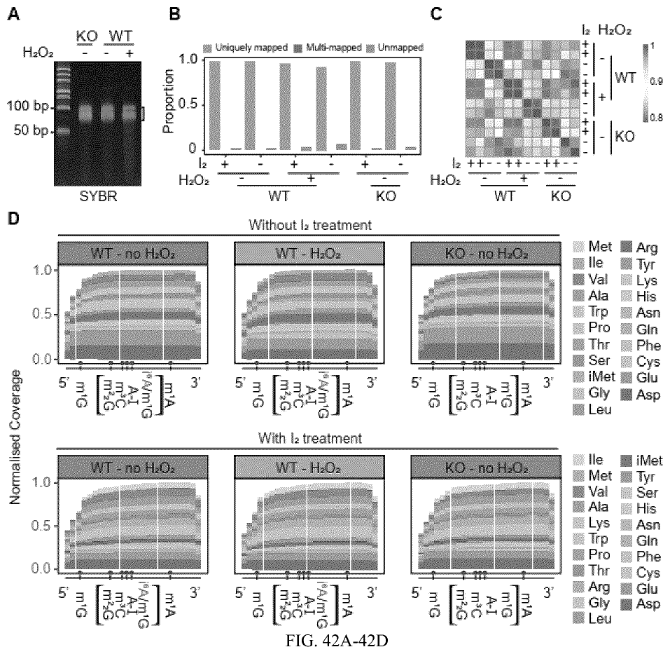

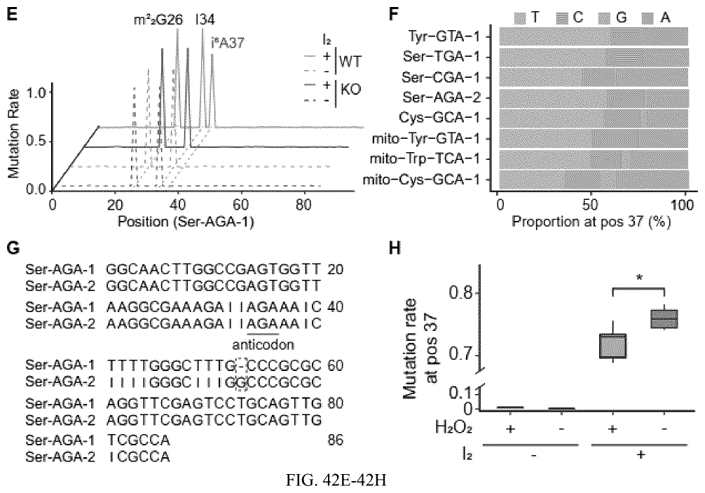

In Saccharomyces cerevisiae, Mod5p is responsible for catalyzing the i⁶A modification at position 37 in six cytosolic (cy) and three mitochondrial (mt) tRNAs. These tRNAs include cy-tRNA-Ser-TGA-1, cy-tRNA-Ser-AGA-1, cy-tRNA-Ser-AGA-2, cy-tRNA-Ser-CGA-1, cy-tRNA-Cys-GCA-1, cy-tRNA-Tyr-GTA-1, mt-tRNA-Tyr-GTA-1, mt-tRNA-Cys-GCA-1, and mt-tRNA-Trp-TCA-1 (45-47). Our mass spectrometry results confirmed that the deletion of MOD5 leads to the complete loss of i⁶A modification in tRNAs (FIGS. 37A and 38A).

To map i⁶A sites and quantify their modification levels, we conducted IMCRT tRNA-seq. Gel-purified tRNAs were treated with or without I₂ before constructing sequencing libraries (FIG. 37B). The quality of these sequencing libraries was assessed using several key parameters. First, over 90% of the total reads uniquely mapped to yeast tRNA sequences (FIG. 38B). Second, the two biological replicates showed high consistency (FIG. 38C). Third, approximately 50% of the reads in all samples were full-length cDNA products (FIG. 38D). Lastly, several known RNA modifications, including m¹A, m³C, m¹G, and A-I editing (FIG. 38D), were readily identified based on mutations induced during reverse transcription (48). Collectively, these results confirm the high quality of our IMCRT tRNA-seq data.

Importantly, our IMCRT tRNA-seq enables the accurate identification of all yeast tRNAs with i⁶A modification. For instance, in wild-type cells, the mutation rate (MR) at A37 in cy-tRNA-Ser-AGA-1 was approximately 0% before I₂ treatment (FIGS. 37C and 38E). The MR was increased to 72%, due to the mutation of A to T, C, or G (FIG. 37D). However, in the MOD5 deletion mutant, MR values remained around 0% both before and after I₂ treatment. We calculated an i⁶A37 score for each tRNA based on the MR at position 37 in all tRNAs. In the wild-type cell, only 9 tRNAs had an i⁶A37 score greater than 1, corresponding to the previously characterized i⁶A37-containing tRNAs (FIGS. 37E and 38F). In the MOD5 deletion mutant, the i⁶A37 scores for these 9 tRNAs dropped to near 0. Furthermore, IMCRT tRNA-seq could distinguish i⁶A37-containing tRNAs with even a single nucleotide difference, such as between cy-tRNA-Ser-AGA-1 and cy-tRNA-Ser-AGA-2 (FIG. 38G), a task challenging for northern blotting-based methods (49). Consistent with previous studies, the 9 i⁶A37-containing tRNAs exhibited a conserved motif around A37 (FIG. 37F), featuring three consecutive adenosines from positions 36 to 38 (50-52). Together, these results demonstrate that IMCRT tRNA-seq can accurately identify i⁶A37-containing tRNAs with single nucleotide resolution.

Next, we conducted IMCRT tRNA-seq experiments on tRNAs extracted from yeast cells cultured under different conditions. Consistent with a previous study, H₂O₂ treatment resulted in decreased tRNA i⁶A levels, as detected by mass spectrometry (FIG. 37G) (38). Notably, H₂O₂ treatment also led to significantly decreases in both MR values and i⁶A37 scores for i⁶A37-containing tRNAs (FIGS. 37H and 38H). Moreover, the average i⁶A37 scores determined by our IMCRT tRNA-seq exhibited a linear correlation with i⁶A levels as determined by mass spectrometry (FIG. 371). These results strongly suggest that our IMCRT tRNA-seq methodology has the potential to semi-quantitatively assess i⁶A37 levels of endogenous RNAs

### Conclusion

In summary, we employed Ene-ligation bioorthogonal chemistry and novel PTAD-based fluorescence probes for the direct labeling of i⁶A-containing RNAs in vitro. We synthesized the i⁶A triphosphate building block and demonstrated that it can be recognized and incorporated by eukaryotic RNA polymerase during intracellular transcription, allowing for adjustable i⁶A concentrations and the production of i⁶A-modified RNA that can be directly labeled by our PTAD-based fluorescent dye (8 and 10). This method enables us to study the functions of i⁶A and provides a general approach to label fluorescence onto RNAs for molecular tracking based on i⁶A addition.

We also discovered that iodine-mediated cyclization reactions of the prenyl group occur rapidly, transforming i⁶A from a hydrogen-bond acceptor to a donor. Leveraging this reactivity, we developed a novel iodine-mediated cyclization and reverse transcription sequencing (termed IMCRT tRNA-seq) method for profiling i⁶A residues in cellular RNAs. Following established protocols, we demonstrated that IMCRT tRNA-seq accurately identifies tRNAs containing i⁶A37 with single nucleotide precision, including all yeast tRNAs with i⁶A modification. Under stress response conditions (eg. H₂O₂), we observed decreased i⁶A levels in budding yeast, accompanied by substantial decreases in both the mutation rate at A37 and i⁶A37 scores. These findings suggest that our IMCRT tRNA-seq technique is not only capable of mapping i⁶A sites with single nucleotide resolution, but also permits semi-quantification of i⁶A levels in tRNAs.

In addition to i⁶A, over 100 RNA modifications have been identified. Accurate mapping and quantification methods are essential for studying their biological roles. While antibodies recognition of some specific modifications like m⁶A, m¹A, m⁷G, and ac⁴C have been used for mapping, their resolution is typically limited to tens or hundreds of nucleotides due to fragmented RNA sizes. Alternatively, methods combining unique chemical reactivity and detection of mutation/stop caused during reverse transcription have successfully identified a few modifications such as m⁵C, pseudouridine (Ψ) and ac⁴C at single-base resolution (53). However, low abundance modifications require increased sequencing depth or pre-library construction enrichment using antibodies, as in m¹A-MAP methods. In budding yeast, the high proportion of i⁶A-containing tRNAs negates the need for enrichment, but detecting i⁶A in other RNA species might necessitate enrichment using anti-i⁶A antibodies (54-55).

While there is currently no clear evidence for the presence and distribution of i⁶A residues across the transcriptome, likely due to the lack of appropriate molecular tools, the question of whether this prenyl modification exists in other RNA species such as mRNA remains open. Nonetheless, i⁶A residues have been actively implicated in various human diseases, including diabetes, cancer, and neurodegenerative disorders. Moreover, the biological functions of prenyl modifications in both RNA and proteins have gained increasing recognition. For instance, the expression levels of prenylated 2′-5′-oligoadenylate synthetase 1 (OAS1) have been closely associated with the severity of hospitalized SARS-COV-2 patients, highlighting the potential critical roles of prenylation in the human antiviral defense system (56). Our newly developed chemical biology methods will offer unique toolsets for detecting, profiling, and monitoring i⁶A residues in both normal and diseased cell environments, facilitating more comprehensive functional studies of this natural modification and shedding light on its implications in various biological processes and pathologies.

### REFERENCES

1. Nachtergaele, S. and He, C. (2018) Chemical Modifications in the Life of an mRNA Transcript. Annu Rev Genet, 52, 349-372.
2. Bhat, S. A., Ahmad, S. M., Mumtaz, P. T., Malik, A. A., Dar, M. A., Urwat, U., Shah, R. A. and Ganai, N. A. (2016) Long non-coding RNAs: Mechanism of action and functional utility. Noncoding RNA Res, 1, 43-50.
3. Qu, S., Yang, X., Li, X., Wang, J., Gao, Y., Shang, R., Sun, W., Dou, K. and Li, H. (2015) Circular RNA: A new star of noncoding RNAs. Cancer Lett, 365, 141-148.
4. Roundtree, I. A., Evans, M. E., Pan, T. and He, C. (2017) Dynamic RNA Modifications in Gene Expression Regulation. Cell, 169, 1187-1200.
5. Kim, J. and Franco, E. (2020) RNA nanotechnology in synthetic biology. Curr Opin Biotechnol, 63, 135-141.
6. Sletten, E. M. and Bertozzi, C.R. (2009) Bioorthogonal chemistry: fishing for selectivity in a sea of functionality. Angew Chem Int Ed Engl, 48, 6974-6998.
7. King, M. and Wagner, A. (2014) Developments in the field of bioorthogonal bond forming reactions-past and present trends. Bioconjug Chem, 25, 825-839.
8. Machnicka, M. A., Milanowska, K., Osman Oglou, O., Purta, E., Kurkowska, M., Olchowik, A., Januszewski, W., Kalinowski, S., Dunin-Horkawicz, S., Rother, K. M. et al. (2013) MODOMICS: a database of RNA modification pathways—2013 update. Nucleic Acids Res, 41, D262-267.
9. Leung, H. C., Chen, Y. and Winkler, M. E. (1997) Regulation of substrate recognition by the MiaA tRNA prenyltransferase modification enzyme of Escherichia coli K-12. J Biol Chem, 272, 13073-13083.
10. Chimnaronk, S., Forouhar, F., Sakai, J., Yao, M., Tron, C. M., Atta, M., Fontecave, M., Hunt, J. F. and Tanaka, I. (2009) Snapshots of dynamics in synthesizing N⁶-isopentenyladenosine at the tRNA anticodon. Biochemistry, 48, 5057-5065.
11. Cheng, H. P., Yang, X. H., Lan, L., Xie, L. J., Chen, C., Liu, C., Chu, J., Li, Z. Y., Liu, L., Zhang, T. Q. et al. (2020) Chemical Deprenylation of N⁶-Isopentenyladenosine (i⁶A) RNA. Angew Chem Int Ed Engl, 59, 10645-10650.
12. Petrullo, L. A., Gallagher, P. J. and Elseviers, D. (1983) The role of 2-methylthio-N⁶-isopentenyladenosine in readthrough and suppression of nonsense codons in Escherichia coli. Mol Gen Genet, 190, 289-294.
13. Connolly, D. M. and Winkler, M. E. (1989) Genetic and physiological relationships among the miaA gene, 2-methylthio-N⁶-(delta 2-isopentenyl)-adenosine tRNA modification, and spontaneous mutagenesis in Escherichia coli K-12. J Bacteriol, 171, 3233-3246.
14. Urbonavicius, J., Qian, Q., Durand, J. M., Hagervall, T. G. and Bjork, G. R. (2001) Improvement of reading frame maintenance is a common function for several tRNA modifications. EMBO J, 20, 4863-4873.
15. Jonkhout, N., Tran, J., Smith, M. A., Schonrock, N., Mattick, J. S. and Novoa, E. M. (2017) The RNA modification landscape in human disease. RNA, 23, 1754-1769.
16. Dumelin, C. E., Chen, Y., Leconte, A. M., Chen, Y. G. and Liu, D. R. (2012) Discovery and biological characterization of geranylated RNA in bacteria. Nat Chem Biol, 8, 913-919.
17. Wang, R., Vangaveti, S., Ranganathan, S. V., Basanta-Sanchez, M., Haruehanroengra, P., Chen, A. and Sheng, J. (2016) Synthesis, base pairing and structure studies of geranylated RNA. Nucleic Acids Res, 44, 6036-6045.
18. Wang, R., Ranganathan, S. V., Basanta-Sanchez, M., Shen, F., Chen, A. and Sheng, J. (2015) Synthesis and base pairing studies of geranylated 2-thiothymidine, a natural variant of thymidine. Chem Commun (Camb), 51, 16369-16372.
19. Jager, G., Chen, P. and Bjork, G. R. (2016) Transfer RNA Bound to MnmH Protein Is Enriched with Geranylated tRNA—A Possible Intermediate in Its Selenation? PLoS One, 11, e0153488.
20. Kierzek, E. and Kierzek, R. (2003) The thermodynamic stability of RNA duplexes and hairpins containing N⁶-alkyladenosines and 2-methylthio-N⁶-alkyladenosines. Nucleic Acids Res, 31, 4472-4480.
21. Kierzek, E. and Kierzek, R. (2003) The synthesis of oligoribonucleotides containing N⁶-alkyladenosines and 2-methylthio-N⁶-alkyladenosines via post-synthetic modification of precursor oligomers. Nucleic Acids Res, 31, 4461-4471.
22. Liaqat, A., Stiller, C., Michel, M., Sednev, M. V. and Hobartner, C. (2020) N⁶-Isopentenyladenosine in RNA Determines the Cleavage Site of Endonuclease Deoxyribozymes. Angew Chem Int Ed Engl, 59, 18627-18631.
23. Arimbasseri, A. G., Iben, J., Wei, F. Y., Rijal, K., Tomizawa, K., Hafner, M. and Maraia, R. J. (2016) Evolving specificity of tRNA 3-methyl-cytidine-32 (m³C32) modification: a subset of tRNAs_{Ser} requires N⁶-isopentenylation of A37. RNA, 22, 1400-1410.
24. Han, L., Marcus, E., D'Silva, S. and Phizicky, E. M. (2017) S. cerevisiae Trm140 has two recognition modes for 3-methylcytidine modification of the anticodon loop of tRNA substrates. RNA, 23, 406-419.
25. Oslovsky, V. E., Drenichev, M. S. and Mikhailov, S. N. (2015) Regioselective 1-N-Alkylation and Rearrangement of Adenosine Derivatives. Nucleosides Nucleotides Nucleic Acids, 34, 475-499.
26. Fang, Z., Lu, Z., Han, S., Zhou, Y., Yang, W., Zhang, X. and Zhou, X. (2023) The Transcriptome-Wide Mapping of 2-Methylthio-N⁶-isopentenyladenosine at Single-Base Resolution. J Am Chem Soc, 145, 5467-5473.
27. Jia, G., Fu, Y., Zhao, X., Dai, Q., Zheng, G., Yang, Y., Yi, C., Lindahl, T., Pan, T., Yang, Y. G. et al. (2011) N⁶-methyladenosine in nuclear RNA is a major substrate of the obesity-associated FTO. Nat Chem Biol, 7, 885-887.
28. Zeng, H., He, B., Xia, B., Bai, D., Lu, X., Cai, J., Chen, L., Zhou, A., Zhu, C., Meng, H. et al. (2018) Bisulfite-Free, Nanoscale Analysis of 5-Hydroxymethylcytosine at Single Base Resolution. J Am Chem Soc, 140, 13190-13194.
29. Wang, Y., Zhang, X., Wu, F., Chen, Z. and Zhou, X. (2019) Bisulfite-free, single base-resolution analysis of 5-hydroxymethylcytosine in genomic DNA by chemical-mediated mismatch. Chem Sci, 10, 447-452.
30. Xia, B., Han, D., Lu, X., Sun, Z., Zhou, A., Yin, Q., Zeng, H., Liu, M., Jiang, X., Xie, W. et al. (2015) Bisulfite-free, base-resolution analysis of 5-formylcytosine at the genome scale. Nat Methods, 12, 1047-1050.
31. Li, X., Zhu, P., Ma, S., Song, J., Bai, J., Sun, F. and Yi, C. (2015) Chemical pulldown reveals dynamic pseudouridylation of the mammalian transcriptome. Nat Chem Biol, 11, 592-597.
32. Zhang, J. W., Zhao, J. Z., Wang, L., Hu, H. M., Wang, S., Yu, P., Wang, R. (2021) Labeling of polyprenylated macromolecules using mild Ene reactions. Tetrahedron Lett, 81, 131917.
33. Zhang, J. W., Li, Y. Y., Wang, S., Wang, R. (2021) Labeling of prenylated nucleic acid by Ene-type fluorination under physiological condition. Tetrahedron Lett, 74, 153162.
34. Wang, S., Li, Y. Y., Zhou, H. L., Wang, L. and Wang, R. (2022) Development of Biocompatible Ene-Ligation Enabled by Prenyl-Based beta-Caryophyllene with Triazoline/Selectfluor under Physiological Conditions. J Org Chem, 87, 8648-8655.
35. Luo, H. Q., Loh, T. P. (2009) Synthesis of aryl allylic fluorides by direct electrophilic fluorination of alkenes. Tetrahedron Lett, 50, 1554-1556.
36. Coelho, J. A. S., Matsumoto, A., Orlandi, M., Hilton, M. J., Sigman, M. S. and Toste, F. D. (2018) Enantioselective fluorination of homoallylic alcohols enabled by the tuning of non-covalent interactions. Chem Sci, 9, 7153-7158.
37. Al Shuhaib, Z., Davies, D. H., Dennis, M., Evans, D. M., Fletcher, M. D., Franken, H., Hancock, P., Hollinshead, J., Jones, I., Kahm, K., Murphy, P. J., Nash, R., Potter, D., Rowles, R. (2014) Iodocyclisations reactions of Boc- and Cbz-protected N-allylguanidines. Tetrahedron Lett, 70, 4412-4419.
38. Chan, C. T., Dyavaiah, M., DeMott, M. S., Taghizadeh, K., Dedon, P. C. and Begley, T. J. (2010) A quantitative systems approach reveals dynamic control of tRNA modifications during cellular stress. PLoS Genet, 6, e1001247.
39. Behrens, A., Rodschinka, G., Nedialkova. High-resolution quantitative profiling of tRNA abundance and modification status in eukaryotes by mim-tRNAseq. (2021) Mol Cell. 81, 1802-1815.e7.
40. Martin, M. Cutadapt removes adapter sequences from high-throughput sequencing reads. (2011) EMBnet j. 17, 10.
41. Clark, W. C., Evans, M. E., Dominissini, D., Zheng, G. and Pan, T. (2016) tRNA base methylation identification and quantification via high-throughput sequencing. RNA, 22, 1771-1784.
42. Suazo, K. F., Park, K. Y. and Distefano, M. D. (2021) A Not-So-Ancient Grease History: Click Chemistry and Protein Lipid Modifications. Chem Rev, 121, 7178-7248.
43. Rashidian, M., Kumarapperuma, S.C., Gabrielse, K., Fegan, A., Wagner, C. R. and Distefano, M. D. (2013) Simultaneous dual protein labeling using a triorthogonal reagent. J Am Chem Soc, 135, 16388-16396.
44 Nyffeler, P. T., Duron, S. G., Burkart, M. D., Vincent, S. P. and Wong, C. H. (2004) Selectfluor: mechanistic insight and applications. Angew Chem Int Ed Engl, 44, 192-212.
45. Shu, X., Dai, Q., Wu, T., Bothwell, I. R., Yue, Y., Zhang, Z., Cao, J., Fei, Q., Luo, M., He, C. et al. (2017) N⁶-Allyladenosine: A New Small Molecule for RNA Labeling Identified by Mutation Assay. J Am Chem Soc, 139, 17213-17216.
46. Shu, X., Cao, J., Cheng, M., Xiang, S., Gao, M., Li, T., Ying, X., Wang, F., Yue, Y., Lu, Z. et al. (2020) A metabolic labeling method detects m⁶A transcriptome-wide at single base resolution. Nat Chem Biol, 16, 887-895.
47. Bai, M., Cao, X., Huang, W., Chen, F., Zhao, Y., Xue, J., Li, Y., Cheng, Y., Zhang, L. and Zhao, Y. (2020) Visualizing Newly Synthesized RNA by Bioorthogonal Labeling-Primed DNA Amplification. Anal Chem, 92, 8444-8449.
48. Lamichhane, T. N., Arimbasseri, A. G., Rijal, K., Iben, J. R., Wei, F. Y., Tomizawa, K. and Maraia, R. J. (2016) Lack of tRNA-i⁶A modification causes mitochondrial-like metabolic deficiency in S. pombe by limiting activity of cytosolic tRNA_{Tyr}, not mito-RNA. RNA, 22, 583-596.
49. Yarham, J. W., Lamichhane, T. N., Pyle, A., Mattijssen, S., Baruffini, E., Bruni, F., Donnini, C., Vassilev, A., He, L., Blakely, E. L. et al. (2014) Defective i⁶A37 modification of mitochondrial and cytosolic tRNAs results from pathogenic mutations in TRIT1 and its substrate tRNA. PLoS Genet, 10, e1004424.
50. Schweizer, U., Bohleber, S. and Fradejas-Villar, N. (2017) The modified base isopentenyladenosine and its derivatives in tRNA. RNA Biol, 14, 1197-1208.
51. Khalique, A., Mattijssen, S., Haddad, A. F., Chaudhry, S. and Maraia, R. J. (2020) Targeting mitochondrial and cytosolic substrates of TRIT1 isopentenyltransferase: Specificity determinants and tRNA-i⁶A37 profiles. PLoS Genet, 16, e1008330.
52. Lamichhane, T. N., Blewett, N. H. and Maraia, R. J. (2011) Plasticity and diversity of tRNA anticodon determinants of substrate recognition by eukaryotic A37 isopentenyltransferases. RNA, 17, 1846-1857.
53. Zhang, M., Xiao, Y., Jiang, Z., Yi, C. (2023) Quantifying m⁶A and Ψ modifications in the transcriptome via chemical-assisted approaches. Acc Chem Res. 56 (21), 2980-2991.
54. Senapathy, P., Jacob, M. T. (1981) Identification and purification of tRNAs containing N⁶-(delta 2-isopentenyl) adenosine using antibodies specific for N⁶-(delta-isopentenyl) adenosine. J Biol Chem. 256 (22), 11580-4.
55. Faust, J. R., Dice, J. F. (1991) Evidence for isopentenyladenine modification on a cell cycle-regulated protein. J Biol Chem. 266 (15), 9961-70.
56. Wickenhagen, A., Sugrue, E., Lytras, S., Kuchi, S., Noerenberg, M., Turnbull, M. L., Loney, C., Herder, V., Allan, J., Jarmson, I. et al. (2021) A prenylated dsRNA sensor protects against severe COVID-19. Science, 374, eabj3624.

Although some non-limiting examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

What is claimed is:

1. A compound of Formula I: 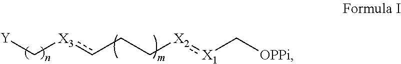
wherein
X₁ is —C(═O)— or —CH₂—,
each [symbol] is, independently, a single bond or a double bond,
X₂ is —NH— or —C(CH₃)—,
m is 0 or 1,
X₃ is —CH₂— or —C(CH₃)—,
n is an integer of from 0 to 8, and
Y is N₃ or 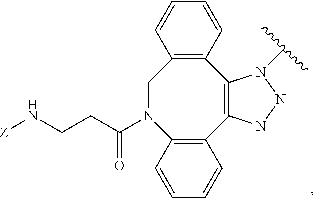
wherein Z is a fluorescent label.

2. The compound of claim 1, wherein n is an integer of from 1 to 8.

3. The compound of claim 1, wherein m is 1.

4. The compound of claim 1, wherein m is 1 and n is 8.

5. The compound of claim 1, wherein m is 0 and n is 1.

6. The compound of claim 1, wherein each of X₂ and X₃ is —C(CH₃)— and each [symbol] is a double bond.

7. The compound of claim 1, wherein
X₁ is —CH₂—, X₂ is —C(CH₃)—, each [symbol] is a double bond, m is 1, X₃ is —C(CH₃)—, and n is 1, or
X₁ is —C(═O)—, X₂ is —NH—, each [symbol] is a single bond, m is 0, X₃ is —CH₂—, and n is 1.

8. The compound of claim 1, wherein Z is a fluorescent label selected from Cy5, FITC, and BODIPY.

9. A method of forming a tagged tRNA, comprising
contacting a 2-thiouridine tRNA with a compound of Formula I: 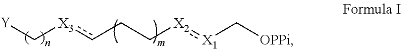
wherein
X₁ is —C(═O)— or —CH₂—,
each [symbol] is, independently, a single bond or a double bond,
X₂ is —NH— or —C(CH₃)—,
m is 0 or 1,
X₃ is —CH₂— or —C(CH₃)—,
n is an integer of from 0 to 8, and
Y is an azide or 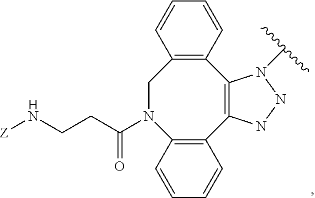
wherein Z is a fluorescent label; and
a step for covalently attaching the compound of Formula I to the 2-thiouridine tRNA.

10. The method of claim 9, wherein n is an integer of from 1 to 8.

11. The method of claim 9, wherein m is 1.

12. The method of claim 9, wherein m is 1 and n is 8.

13. The method of claim 9, wherein m is 0 and n is 1.

14. The method of claim 8, wherein each of X₂ and X₃ is —C(CH₃)— and each [symbol] is a double bond.

15. The method of claim 9, wherein
X₁ is —CH₂—, X₂ is —C(CH₃)—, each [symbol] is a double bond, m is 1, X₃ is —C(CH₃)—, and n is 1, or
X₁ is —C(═O)—, X₂ is —NH—, each [symbol] is a single bond, m is 0, X₃ is —CH₂—, and n is 1.

16. The method of claim 9, wherein Y is an azide, and forming the tagged tRNA further comprises contacting the azide with a DBCO-activated fluorescent label to cause a click-chemistry reaction therebetween.

17. The method of claim 9, wherein Y is 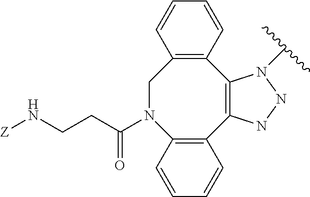
and Z is a fluorescent label.

18. The method of claim 9, wherein the step for covalently attaching the compound of Formula I to the 2-thiouridine tRNA occurs intracellularly.

19. The method of claim 9, wherein the step for covalently attaching the compound of Formula I to the 2-thiouridine tRNA occurs extracellularly.

20. The method of claim 9, wherein Z is a fluorescent label selected from Cy5, FITC, and BODIPY.
